(19) United States
(12) Patent Application Publication
(10) Pub. No.: US 2025/0382354 A1
(43) Pub. Date: Dec. 18, 2025

(54) CROSS-SPECIFIC ANTIBODIES, USES AND METHODS FOR DISCOVERY THEREOF

(71) Applicant: INSTITUTE FOR RESEARCH IN BIOMEDICINE (IRB), Bellinzona (CH)

(72) Inventors: Davide ROBBIANI, Massagno (CH); Filippo BIANCHINI, Luisago (Como) (IT); Virginia CRIVELLI, Bellinzona (CH); Andrea CAVALLI, Zürich (CH); Luca VARANI, Gorduno (CH)

(21) Appl. No.: 18/872,666

(22) PCT Filed: Jun. 8, 2023
(86) PCT No.: PCT/EP2023/065432

§ 371 Date: Dec. 6, 2024

(30) Foreign Application Priority Data
Jun. 8, 2022 (EP) 22177974.7
Jan. 16, 2023 (EP) 23151851.5

(51) Int. Cl. C07K 16/10; A61P 31/14; C12N 15/11; G01N 15/149; G01N 33/543; G01N 33/563

(52) U.S. Cl. CPC C07K 16/1003; A61P 31/14; C12N 15/11; G01N 15/149; G01N 33/54366; G01N 33/563; C07K 2317/31; C07K 2317/33; C07K 2317/565; C07K 2317/92

(57) ABSTRACT

The invention relates to antibodies, multispecific antibodies, or antigen-binding fragments thereof, specifically binding to a conserved region of a coronavirus S protein and their use in medicine, such as, in the treatment and/or prevention of a coronavirus infection. The invention further relates to methods for identifying coldspot antibodies or antigen-binding fragments thereof.

### REFERENCE TO ELECTRONIC SEQUENCE LISTING

The application contains a Sequence Listing which has been submitted electronically in .XML format and is hereby incorporated by reference in its entirety. Said .XML copy, created on Jul. 23, 2025, is named “VOS-137US.xml” and is 507,241 bytes in size. The sequence listing contained in this .XML file is part of the specification and is hereby incorporated by reference herein in its entirety.

The invention relates to antibodies, multispecific antibodies, or antigen-binding fragments thereof, specifically binding to a conserved region of a coronavirus S protein and their use in medicine, such as, in the treatment and/or prevention of a coronavirus infection. The invention further relates to methods for identifying coldspot antibodies or antigen-binding fragments thereof.

Mutating target antigens are a major problem in therapeutic antibody development and therapy. During the time from the discovery to the development of an antibody and its use in the clinics, target antigen can change and render the antibody obsolete. In addition, the selective pressure induced by therapeutics can favor mutations in the target site pathogens and/or cancers which limits their long-term use.

For example, for the treatment of coronavirus infections, the Spike protein was identified as a therapeutic target. The coronavirus (CoV) Spike protein (S) is a trimeric glycoprotein of S1-S2 heterodimers that mediates binding to target cells and membrane fusion. Most Severe acute respiratory syndrome coronavirus 2 (SARS-CoV-2) neutralizing antibodies that were described to date, target the receptor binding and N-terminal domains of S (RBD and NTD). However, mutations in the viral genome, such as those found in SARS-COV-2 variants of concern (VOC), cause amino acid (aa) changes in the RBD and NTD that diminish or abrogate the effectiveness of vaccines and antiviral monoclonal antibodies that are currently in the clinic.

Innovative approaches are needed to identify countermeasures that remain effective in spite of antigen mutations, such as SARS-COV-2 viral evolution.

Thus, there is a need for means and methods to cross-specifically inhibit targets, in particular coronavirus proteins.

The above technical problem is solved by the embodiments disclosed herein and as defined in the claims.

Accordingly, the invention relates to, inter alia, the following embodiments:
1. An antibody, or antigen-binding fragment thereof, specifically binding to a conserved region of a coronavirus S protein, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.
2. The antibody, or antigen-binding fragment thereof, of embodiment 1, wherein the conserved region is at least one selected from the group consisting of: SEQ ID NO: 01, SEQ ID NO: 02, SEQ ID NO: 16 and SEQ ID NO: 298.
3. The antibody, or antigen-binding fragment thereof, of embodiment 2, the antibody, or antigen-binding fragment thereof, comprising:
   a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
   b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
   c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
   d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
   e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
   f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
   g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
   h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
   i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
   j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
   k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
   l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
   m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
   n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
   o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
   p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
   q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
   r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
   s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
   t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
   u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
   v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
   w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
   x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
   y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
   z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
   aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
   ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
   ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 as defined in SEQ ID NO: 161 and CDR3 as defined in SEQ ID NO: 162; ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
   ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
   af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
   ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
   ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
   ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
   aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
   ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
   al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
   am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
   an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
   ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
   ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
   aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
   ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
   as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
   at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
   au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
   av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
   aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
   ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
   ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
   az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
   ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
   bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; or
   bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451.
4. The antibody, or antigen-binding fragment thereof, of embodiment 3, the antibody, or antigen-binding fragment thereof, comprising a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51.
5. The antibody, or antigen-binding fragment thereof, of embodiment 3, the antibody, or antigen-binding fragment thereof, comprising a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46.
6. The antibody, or antigen-binding fragment thereof, of embodiment 3, the antibody, or antigen-binding fragment thereof, comprising a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96.
7. The antibody, or antigen-binding fragment thereof, of embodiment 2 or 3, wherein the antibody, or antigen-binding fragment thereof, is cross-specific with the S proteins of SARS-COV-2, MERS and HCOV-229E.
8. The antibody, or antigen-binding fragment thereof, of embodiment 2 or 3, wherein the conserved region is SEQ ID NO: 01 and wherein the antibody, or antigen-binding fragment thereof, is cross-specific for coronaviruses of the alpha, beta, gamma and delta genera.
9. The antibody, or antigen-binding fragment thereof, of embodiment 2 or 3, wherein the conserved region is SEQ ID NO: 02 and wherein the antibody, or antigen-binding fragment thereof, is cross-specific for coronaviruses of the alpha, beta and gamma genera.
10. The antibody, or antigen-binding fragment thereof, of embodiment 2 or 3, wherein a) the conserved region is SEQ ID NO: 16 and/or SEQ ID NO: 298; and b) wherein the antibody, or antigen-binding fragment thereof, is cross-specific for SARS-COV-2 variants.
11. A multispecific antibody or a multispecific antigen-binding fragment thereof, specifically binding to at least two regions of a coronavirus S protein, wherein at least one of the coronavirus S protein is a conserved region, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.
12. The multispecific antibody or a multispecific antigen-binding fragment thereof, according to embodiment 11, wherein the at least two regions of the coronavirus S protein comprise an RBD region of a coronavirus.
13. The multispecific antibody or the multispecific antigen-binding fragment thereof, of any one of embodiment 10 to 12, comprising at least one selected from the group of:
   a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
   b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
   c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
   d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
   e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
   f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
   g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
   h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
   i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
   j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
   k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
   l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
   m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
   n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
   o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
   p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
   q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
   r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
   s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
   t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
   u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
   v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
   w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
   x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
   y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
   z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
   aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
   ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
   ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 as defined in SEQ ID NO: 161 and CDR3 as defined in SEQ ID NO: 162;
   ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
   ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
   af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
   ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
   ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
   ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
   aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
   ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
   al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
   am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
   an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
   ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
   ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
   aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
   ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
   as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
   at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
   au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
   av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
   aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
   ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
   ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
   az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
   ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
   bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; and
   bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451;
preferably a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167.
14. A polynucleotide encoding an antibody, or an antigen-binding fragment thereof, according to any one of embodiments 1 to 13.
15. A host cell comprising the polynucleotide of embodiment 14.
16. A method for producing an antibody comprising culturing the host cell of embodiment 15.
17. The antibody, or antigen-binding fragment thereof, of any one of embodiments 1 to 13, the polynucleotide of embodiment 14 or the host cell of embodiment 15 for use as a medicament.
18. A pharmaceutical composition comprising at least two antibodies, or antigen-binding fragments thereof, wherein a first antibody, or antigen-binding fragment thereof is the antibody, or antigen-binding fragment thereof, of any one of embodiments 1 to 10, and wherein a second antibody or antigen fragment thereof, specifically binds to RBD.
19. The antibody, or antigen-binding fragment thereof, of any one of embodiments 1 to 13, the polynucleotide of embodiment 14, the host cell of embodiment 15 or the pharmaceutical composition of embodiment 18 for use in the treatment and/or prevention of a coronavirus infection.
20. A method for treatment and/or prevention of a coronavirus infection in a subject, the method comprising delivering a therapeutically effective amount of the antibody, or antigen-binding fragment thereof, of any one of embodiments 1 to 13, the polynucleotide of embodiment 14, the host cell of embodiment 15 or the pharmaceutical composition of embodiment 18 to a subject.
21. The antibody, or antigen-binding fragment thereof, for use of embodiment 19, the polynucleotide for use of embodiment 19, the host cell for use of embodiment 19 or the pharmaceutical composition for use of embodiment 18 or the method for treatment and/or prevention of embodiment 19, wherein the coronavirus is SARS-CoV-2.
22. A method for identifying a coldspot antibody or an antigen-binding fragment thereof, the method comprising the steps of:
   a) identifying a conserved region in an antigen;
   b) generating a peptide comprising or consisting of the conserved region of the antigen or a fragment of the conserved region;
   c) bringing at least two antibodies or antigen-binding fragments thereof of a convalescent subject into contact with the conserved region, wherein the convalescent subject is a subject that was previously exposed to the antigen; and
   d) identifying at least one coldspot antibody or antigen-binding fragment thereof from the antibodies of the convalescent subject, wherein the coldspot antibody or antigen-binding fragments thereof has at least one better coldspot binding property than at least one second antibody or antigen-binding fragment thereof of the antibodies or antigen-binding fragments thereof of the convalescent subject, wherein a better coldspot binding property is a property selected from the group consisting of:
      i) higher binding affinity to the peptide;
      ii) cross-specificity to the peptide and to a second antigen, wherein the second antigen is a related antigen; and
      iii) specific binding to the peptide and inhibition of a biologic activity of the antigen.
23. The method of embodiment 22, wherein identifying conserved regions of an antigen comprises comparison of a sequence of the antigen to a sequence of a related antigen.
24. The method of embodiment 22 or 23, wherein the conserved region comprises at least 17 consecutive aa with frequency of substitutions <0.1%.
25. The method of any one of embodiments 22 to 24, wherein step c) comprises bringing at least two antibodies or antigen-binding fragments thereof of a convalescent subject into contact with the conserved region in an ELISA.
26. The method of any one of embodiments 22 to 25, wherein step d) comprises fluorescence-activated cell sorting.
27. The method of any one of embodiments 22 to 26, wherein the antibodies or antigen-binding fragments thereof of a convalescent subject are memory B cell-derived antibodies.
28. The method of any one of embodiments 22 to 27, wherein identifying a conserved region further comprises identifying a secondary, tertiary and/or quaternary structure of the conserved region in the antigen.
29. The method of any one of embodiments 22 to 28, wherein the antigen is a pathogen and wherein the convalescent subject is a subject that was previously exposed to the pathogen, a phylogenetically related pathogen, a vaccine to the pathogen and/or or an attenuated and/or inactivated version of the pathogen or to a phylogenetically related pathogen.
30. The method of any one of embodiments 22 to 28, wherein the antigen is a cancer antigen.
31. An antibody, or antigen-binding fragment thereof, specifically binding to the RBD region of a coronavirus S protein, the antibody or antigen-binding fragment thereof comprising:
   a) a variable heavy (VH) chain comprising CDR1 as defined in SEQ ID NO: 173 CDR2 as defined in SEQ ID NO: 174 and CDR3 as defined in SEQ ID NO: 175 and a variable light (VL) chain comprising CDR1 as defined in SEQ ID NO: 176, CDR2 comprising or consisting of the sequence of DAS and CDR3 as defined in SEQ ID NO: 177;
   b) a variable heavy (VH) chain comprising CDR1 as defined in SEQ ID NO: 178, CDR2 as defined in SEQ ID NO: 179 and CDR3 as defined in SEQ ID NO: 180 and a variable light (VL) chain comprising CDR1 as defined in SEQ ID NO: 181, CDR2 comprising or consisting of the sequence of GAS and CDR3 as defined in SEQ ID NO: 182;
   c) a variable heavy (VH) chain comprising CDR1 as defined in SEQ ID NO: 183, CDR2 as defined in SEQ ID NO: 184 and CDR3 as defined in SEQ ID NO: 185 and a variable light (VL) chain comprising CDR1 as defined in SEQ ID NO: 186, CDR2 comprising or consisting of the sequence of EVS and CDR3 as defined in SEQ ID NO: 187;
   d) a variable heavy (VH) chain comprising CDR1 as defined in SEQ ID NO: 188, CDR2 as defined in SEQ ID NO: 189 and CDR3 as defined in SEQ ID NO: 190 and a variable light (VL) chain comprising CDR1 as defined in SEQ ID NO: 191, CDR2 comprising or consisting of the sequence of GNN and CDR3 as defined in SEQ ID NO: 192;
   e) a variable heavy (VH) chain comprising CDR1 as defined in SEQ ID NO: 193, CDR2 as defined in SEQ ID NO: 194 and CDR3 as defined in SEQ ID NO: 195 and a variable light (VL) chain comprising CDR1 as defined in SEQ ID NO: 196, CDR2 comprising or consisting of the sequence of LGS and CDR3 as defined in SEQ ID NO: 197;
   f) a variable heavy (VH) chain comprising CDR1 as defined in SEQ ID NO: 198, CDR2 as defined in SEQ ID NO: 199 and CDR3 as defined in SEQ ID NO: 200 and a variable light (VL) chain comprising CDR1 as defined in SEQ ID NO: 201, CDR2 comprising or consisting of the sequence of EDN and CDR3 as defined in SEQ ID NO: 202;
   g) a variable heavy (VH) chain comprising CDR1 as defined in SEQ ID NO: 203, CDR2 as defined in SEQ ID NO: 204 and CDR3 as defined in SEQ ID NO: 205 and a variable light (VL) chain comprising CDR1 as defined in SEQ ID NO: 206, CDR2 comprising or consisting of the sequence of GAS and CDR3 as defined in SEQ ID NO: 207;
   h) a variable heavy (VH) chain comprising CDR1 as defined in SEQ ID NO: 208, CDR2 as defined in SEQ ID NO: 209 and CDR3 as defined in SEQ ID NO: 210 and a variable light (VL) chain comprising CDR1 as defined in SEQ ID NO: 211, CDR2 comprising or consisting of the sequence of AAS and CDR3 as defined in SEQ ID NO: 212; or
   i) a variable heavy (VH) chain comprising CDR1 as defined in SEQ ID NO: 213, CDR2 as defined in SEQ ID NO: 214 and CDR3 as defined in SEQ ID NO: 215 and a variable light (VL) chain comprising CDR1 as defined in SEQ ID NO: 216, CDR2 comprising or consisting of the sequence of GAS and CDR3 as defined in SEQ ID NO: 217.

Accordingly, in one embodiment, the invention relates to an antibody, or antigen-binding fragment thereof, specifically binding to a conserved region of a coronavirus S protein, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.

The term “antibody” is used herein in the broadest sense and encompasses various antibody structures, including but not limited to monoclonal antibodies, polyclonal antibodies, multispecific antibodies (e.g., bispecific antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies, recombinant human antibodies, heterologous antibodies, heterohybrid antibodies or antibodies displayed upon the surface of a phage or displayed upon the surface of a cell (e.g., a chimeric antigen receptor T cell).

The phrase “specifically binding to a conserved region”, as used herein, refers to an antibody or an antigen-binding fragment thereof that is capable of binding to the conserved region with sufficient affinity such that the antibody or antigen-binding fragment thereof is useful as a preventive, diagnostic and/or therapeutic agent for the desired purpose disclosed herein, in particular for use in preventing or treating a coronavirus infection or symptoms thereof.

In certain embodiments, an antibody or antigen-binding fragment that “binds to a region” within a defined sequence of a protein that is identified by mutation analysis, in which amino acids of the protein are mutated, and binding of the antibody to the resulting altered protein (e.g., an altered protein comprising the epitope) is determined to be at least 20% of the binding to unaltered protein. In some embodiments, an antibody or antigen-binding fragment “that binds to a region” within a defined sequence of a protein that is identified by mutation analysis, in which amino acids of the protein are mutated, and binding of the antibody to the resulting altered protein (e.g., an altered protein comprising the epitope) is determined to be at least 30%, at least 40%, at least 50%, at least 60%, at least 70%, at least 80%, or at least 90% of the binding to unaltered protein. In certain embodiments, binding of the antibody or antigen-binding fragment is determined by fluorescence-activated cell sorting, Wester Blot or by a suitable binding assay such as ELISA.

The term “conserved region of a coronavirus S protein”, as used herein, refers to a region on the coronavirus S protein that can be bound by an antibody and is evolutionarily conserved compared to a related coronavirus S Protein. As such, the conserved region of a coronavirus S protein described herein can be a single conserved region of >17 consecutive amino acids or a subdomain of the coronavirus S protein consisting of more than 50%, more than 60%, more than 70% or more than 80%, or more than 90% of coronavirus S protein single conserved regions. The SD1 region is a discontinuously encoded subdomain consisting primarily of coronavirus S protein single conserved regions (see FIG. 1a/b; SEQ ID NO: 16 and SEQ ID NO: 298). The accessibility of the SEQ ID NO: 16 and SEQ ID NO: 298 may depend on protein folding. In some embodiments, the specific binding to a region as defined by SEQ ID NO: 16 and/or SEQ ID NO: 298 described herein refers to binding within the sequences SEQ ID NO: 16 and/or SEQ ID NO: 298 on a peptide comprising or consisting of a sequence as defined by SEQ ID NO: 299. In some embodiments, the conserved region described herein is a conserved region of a coronavirus S protein and is selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.

The term “binding to” as used in the context of the present invention defines a binding (interaction) of at least two “antigen-interaction-sites” with each other. The term “antigen-interaction-site” defines, in accordance with the present invention, a motif of a polypeptide, i.e., a part of the antibody or antigen-binding fragment of the present invention, which shows the capacity of specific interaction with at least one sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298. Said binding/interaction is also understood to define a “specific recognition”. The term “specifically recognizing” means in accordance with this invention that the antibody is capable of specifically interacting with and/or binding to at least two amino acids of sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298, in particular interacting with/binding to at least 2, at least 3, at least 4, at least 6, at least 7, at least 8, at least 9, at least 10, at least 11, at least 12, at least 13, at least 14, at least 15, at least 16 or all amino acids within the amino acid sequence selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.

The term “coronavirus”, as used herein, refers to any virus of the Coronaviridae family, preferably a coronavirus selected from group comprising MERS-COV, SARS-COV and SARS-COV-2 (or any variant thereof). In some embodiments, the coronavirus described herein is a virus selected from the group consisting of alpha genera, beta genera, gamma genera and delta genera, preferably by the beta genera. In some embodiments, the coronavirus described herein is a virus selected from the group consisting of Embecovirus, Hibecovirus, Merbecovirus, Nobecovirus, and Sarbecovirus.

The term “coronavirus S protein”, as used herein, refers to the spike protein of any coronavirus. The positioning and specific sequence is provided in reference to the protein GenBank: QHO60594.1 (see Table 3). However, the person skilled in the art is aware how to identify the corresponding sequence parts in other coronaviruses.

The inventors identified regions in the coronavirus S protein that are less prone to mutate. Without being bound by theory, these regions of S may be under selective pressure to maintain their aa sequence unchanged because they are essential for its function or to maintain proper quaternary structure.

In specific, 15 regions with infrequent aa changes (coldspots, see Methods) were identified: one coldspot includes the S2′ cleavage site and a portion of the fusion peptide (FP, aa 814-838), which is substrate of the TMPRSS2 and Cathepsin proteases; a second one is at the stem helix that precedes the heptad repeat 2 region (HR2, aa 1142-1161); and three coldspots span sequences at the discontinuously encoded subdomain 1 (SD1; FIG. 1a). Both FP and HR2 coldspots are devoid of aa changes in SARS-COV-2 VOC, while changes are rare in SD1 (FIG. 1b-c and FIG. 6a). Accordingly, the invention is at least in part based on the finding that the antibodies or fragments thereof, binding to the conserved regions described herein are more resistant to typical coronavirus mutations.

Accordingly, in one embodiment, the invention relates to an antibody, or antigen-binding fragment thereof, specifically binding to a conserved region of a coronavirus S protein, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16/SEQ ID NO: 298.

In certain embodiments, the invention relates to an antibody, or antigen-binding fragment thereof, specifically binding to a conserved region of a coronavirus S protein, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.

In certain embodiments, the invention relates to the antibody, or antigen-binding fragment thereof, of the invention wherein the conserved region recognized by the antibody, or antigen-binding fragment thereof, is at least one selected from the group consisting of: SEQ ID NO: 01, SEQ ID NO: 02, SEQ ID NO: 16 and SEQ ID NO: 298.

In certain embodiments, the invention relates to the antibody, or antigen-binding fragment thereof, of the invention wherein the conserved region is SEQ ID NO: 01 or SEQ ID NO: 02.

In certain embodiments, the invention relates to the antibody, or antigen-binding fragment thereof, of the invention wherein the conserved region is SEQ ID NO: 01, SEQ ID NO: 16 and/or SEQ ID NO: 298.

In certain embodiments, the invention relates to the antibody, or antigen-binding fragment thereof, of the invention wherein the conserved region is SEQ ID NO: 02, SEQ ID NO: 16 and/or SEQ ID NO: 298.

In certain embodiments, the invention relates to the antibody, or antigen-binding fragment thereof, of the invention wherein the conserved region is SEQ ID NO: 01.

In certain embodiments, the invention relates to the antibody, or antigen-binding fragment thereof, of the invention wherein the conserved region is SEQ ID NO: 16 and/or SEQ ID NO: 298.

In certain embodiments, the invention relates to the antibody, or antigen-binding fragment thereof, of the invention wherein the conserved region is SEQ ID NO: 02.

In certain embodiments, the invention relates to the antibody, or antigen-binding fragment thereof, of the invention, the antibody, or antigen-binding fragment thereof, comprising:
a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 as defined in SEQ ID NO: 161 and CDR3 as defined in SEQ ID NO: 162;
ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; or
bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451.

The term “CDR”, as used herein, relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in Kabat et al., 1991, 5th edn. US Department of Health and Human Services, Public Health Service, NIH.; Chothia, 1987, J. Mol. Biol. 196, 901-917; Chothia, 1989 Nature 342, 877-883.

In certain embodiments, the invention relates to the antibody, or antigen-binding fragment thereof, of the invention, the antibody, or antigen-binding fragment thereof, comprising:
a) a variable heavy (VH) chain sequence comprising the amino acid sequence of SEQ ID NO: 218 or a sequence having at least 85%, 86%, 87%, 88%, 89%, 90%, 91%, 92%, 93%, 94%, 95%, 96%, 97%, 98% or 99% sequence identity to SEQ ID NO: 218; and a variable light (VL) chain sequence comprising the amino acid sequence of SEQ ID NO: 219 or a sequence having at least 85%, 86%, 87%, 88%, 89%, 90%, 91%, 92%, 93%, 94%, 95%, 96%, 97%, 98% or 99% sequence identity to SEQ ID NO: 219;
b) a variable heavy (VH) chain sequence comprising the amino acid sequence of SEQ ID NO: 220 or a sequence having at least 85%, 86%, 87%, 88%, 89%, 90%, 91%, 92%, 93%, 94%, 95%, 96%, 97%, 98% or 99% sequence identity to SEQ ID NO: 220; and a variable light (VL) chain sequence comprising the amino acid sequence of SEQ ID NO: 221 or a sequence having at least 85%, 86%, 87%, 88%, 89%, 90%, 91%, 92%, 93%, 94%, 95%, 96%, 97%, 98% or 99% sequence identity to SEQ ID NO: 221;
c) a variable heavy (VH) chain sequence comprising the amino acid sequence of SEQ ID NO: 222 or a sequence having at least 85%, 86%, 87%, 88%, 89%, 90%, 91%, 92%, 93%, 94%, 95%, 96%, 97%, 98% or 99% sequence identity to SEQ ID NO: 222; and a variable light (VL) chain sequence comprising the amino acid sequence of SEQ ID NO: 223 or a sequence having at least 85%, 86%, 87%, 88%, 89%, 90%, 91%, 92%, 93%, 94%, 95%, 96%, 97%, 98% or 99% sequence identity to SEQ ID NO: 223;
d) a variable heavy (VH) chain sequence comprising the amino acid sequence of SEQ ID NO: 224 or a sequence having at least 85%, 86%, 87%, 88%, 89%, 90%, 91%, 92%, 93%, 94%, 95%, 96%, 97%, 98% or 99% sequence identity to SEQ ID NO: 224; and a variable light (VL) chain sequence comprising the amino acid sequence of SEQ ID NO: 225 or a sequence having at least 85%, 86%, 87%, 88%, 89%, 90%, 91%, 92%, 93%, 94%, 95%, 96%, 97%, 98% or 99% sequence identity to SEQ ID NO: 225;
e) a variable heavy (VH) chain sequence comprising the amino acid sequence of SEQ ID NO: 226 or a sequence having at least 85%, 86%, 87%, 88%, 89%, 90%, 91%, 92%, 93%, 94%, 95%, 96%, 97%, 98% or 99% sequence identity to SEQ ID NO: 226; and a variable light (VL) chain sequence comprising the amino acid sequence of SEQ ID NO: 227 or a sequence having at least 85%, 86%, 87%, 88%, 89%, 90%, 91%, 92%, 93%, 94%, 95%, 96%, 97%, 98% or 99% sequence identity to SEQ ID NO: 227;
f) a variable heavy (VH) chain sequence comprising the amino acid sequence of SEQ ID NO: 228 or a sequence having at least 85%, 86%, 87%, 88%, 89%, 90%, 91%, 92%, 93%, 94%, 95%, 96%, 97%, 98% or 99% sequence identity to SEQ ID NO: 228; and a variable light (VL) chain sequence comprising the amino acid sequence of SEQ ID NO: 229 or a sequence having at least 85%, 86%, 87%, 88%, 89%, 90%, 91%, 92%, 93%, 94%, 95%, 96%, 97%, 98% or 99% sequence identity to SEQ ID NO: 229;
g) a variable heavy (VH) chain sequence comprising the amino acid sequence of SEQ ID NO: 230 or a sequence having at least 85%, 86%, 87%, 88%, 89%, 90%, 91%, 92%, 93%, 94%, 95%, 96%, 97%, 98% or 99% sequence identity to SEQ ID NO: 230; and a variable light (VL) chain sequence comprising the amino acid sequence of SEQ ID NO: 231 or a sequence having at least 85%, 86%, 87%, 88%, 89%, 90%, 91%, 92%, 93%, 94%, 95%, 96%, 97%, 98% or 99% sequence identity to SEQ ID NO: 231;
h) a variable heavy (VH) chain sequence comprising the amino acid sequence of SEQ ID NO: 232 or a sequence having at least 85%, 86%, 87%, 88%, 89%, 90%, 91%, 92%, 93%, 94%, 95%, 96%, 97%, 98% or 99% sequence identity to SEQ ID NO: 232; and a variable light (VL) chain sequence comprising the amino acid sequence of SEQ ID NO: 233 or a sequence having at least 85%, 86%, 87%, 88%, 89%, 90%, 91%, 92%, 93%, 94%, 95%, 96%, 97%, 98% or 99% sequence identity to SEQ ID NO: 233;
i) a variable heavy (VH) chain sequence comprising the amino acid sequence of SEQ ID NO: 234 or a sequence having at least 85%, 86%, 87%, 88%, 89%, 90%, 91%, 92%, 93%, 94%, 95%, 96%, 97%, 98% or 99% sequence identity to SEQ ID NO: 234; and a variable light (VL) chain sequence comprising the amino acid sequence of SEQ ID NO: 235 or a sequence having at least 85%, 86%, 87%, 88%, 89%, 90%, 91%, 92%, 93%, 94%, 95%, 96%, 97%, 98% or 99% sequence identity to SEQ ID NO: 235;
j) a variable heavy (VH) chain sequence comprising the amino acid sequence of SEQ ID NO: 236 or a sequence having at least 85%, 86%, 87%, 88%, 89%, 90%, 91%, 92%, 93%, 94%, 95%, 96%, 97%, 98% or 99% sequence identity to SEQ ID NO: 236; and a variable light (VL) chain sequence comprising the amino acid sequence of SEQ ID NO: 237 or a sequence having at least 85%, 86%, 87%, 88%, 89%, 90%, 91%, 92%, 93%, 94%, 95%, 96%, 97%, 98% or 99% sequence identity to SEQ ID NO: 237;
k) a variable heavy (VH) chain sequence comprising the amino acid sequence of SEQ ID NO: 238 or a sequence having at least 85%, 86%, 87%, 88%, 89%, 90%, 91%, 92%, 93%, 94%, 95%, 96%, 97%, 98% or 99% sequence identity to SEQ ID NO: 238; and a variable light (VL) chain sequence comprising the amino acid sequence of SEQ ID NO: 239 or a sequence having at least 85%, 86%, 87%, 88%, 89%, 90%, 91%, 92%, 93%, 94%, 95%, 96%, 97%, 98% or 99% sequence identity to SEQ ID NO: 239;
l) a variable heavy (VH) chain sequence comprising the amino acid sequence of SEQ ID NO: 240 or a sequence having at least 85%, 86%, 87%, 88%, 89%, 90%, 91%, 92%, 93%, 94%, 95%, 96%, 97%, 98% or 99% sequence identity to SEQ ID NO: 240; and a variable light (VL) chain sequence comprising the amino acid sequence of SEQ ID NO: 241 or a sequence having at least 85%, 86%, 87%, 88%, 89%, 90%, 91%, 92%, 93%, 94%, 95%, 96%, 97%, 98% or 99% sequence identity to SEQ ID NO: 241;
m) a variable heavy (VH) chain sequence comprising the amino acid sequence of SEQ ID NO: 242 or a sequence having at least 85%, 86%, 87%, 88%, 89%, 90%, 91%, 92%, 93%, 94%, 95%, 96%, 97%, 98% or 99% sequence identity to SEQ ID NO: 242; and a variable light (VL) chain sequence comprising the amino acid sequence of SEQ ID NO: 243 or a sequence having at least 85%, 86%, 87%, 88%, 89%, 90%, 91%, 92%, 93%, 94%, 95%, 96%, 97%, 98% or 99% sequence identity to SEQ ID NO: 243;
n) a variable heavy (VH) chain sequence comprising the amino acid sequence of SEQ ID NO: 244 or a sequence having at least 85%, 86%, 87%, 88%, 89%, 90%, 91%, 92%, 93%, 94%, 95%, 96%, 97%, 98% or 99% sequence identity to SEQ ID NO: 244; and a variable light (VL) chain sequence comprising the amino acid sequence of SEQ ID NO: 245 or a sequence having at least 85%, 86%, 87%, 88%, 89%, 90%, 91%, 92%, 93%, 94%, 95%, 96%, 97%, 98% or 99% sequence identity to SEQ ID NO: 245;
o) a variable heavy (VH) chain sequence comprising the amino acid sequence of SEQ ID NO: 246 or a sequence having at least 85%, 86%, 87%, 88%, 89%, 90%, 91%, 92%, 93%, 94%, 95%, 96%, 97%, 98% or 99% sequence identity to SEQ ID NO: 246; and a variable light (VL) chain sequence comprising the amino acid sequence of SEQ ID NO: 247 or a sequence having at least 85%, 86%, 87%, 88%, 89%, 90%, 91%, 92%, 93%, 94%, 95%, 96%, 97%, 98% or 99% sequence identity to SEQ ID NO: 247;
p) a variable heavy (VH) chain sequence comprising the amino acid sequence of SEQ ID NO: 248 or a sequence having at least 85%, 86%, 87%, 88%, 89%, 90%, 91%, 92%, 93%, 94%, 95%, 96%, 97%, 98% or 99% sequence identity to SEQ ID NO: 248; and a variable light (VL) chain sequence comprising the amino acid sequence of SEQ ID NO: 249 or a sequence having at least 85%, 86%, 87%, 88%, 89%, 90%, 91%, 92%, 93%, 94%, 95%, 96%, 97%, 98% or 99% sequence identity to SEQ ID NO: 249;
q) a variable heavy (VH) chain sequence comprising the amino acid sequence of SEQ ID NO: 250 or a sequence having at least 85%, 86%, 87%, 88%, 89%, 90%, 91%, 92%, 93%, 94%, 95%, 96%, 97%, 98% or 99% sequence identity to SEQ ID NO: 250; and a variable light (VL) chain sequence comprising the amino acid sequence of SEQ ID NO: 251 or a sequence having at least 85%, 86%, 87%, 88%, 89%, 90%, 91%, 92%, 93%, 94%, 95%, 96%, 97%, 98% or 99% sequence identity to SEQ ID NO: 251;
r) a variable heavy (VH) chain sequence comprising the amino acid sequence of SEQ ID NO: 252 or a sequence having at least 85%, 86%, 87%, 88%, 89%, 90%, 91%, 92%, 93%, 94%, 95%, 96%, 97%, 98% or 99% sequence identity to SEQ ID NO: 252; and a variable light (VL) chain sequence comprising the amino acid sequence of SEQ ID NO: 253 or a sequence having at least 85%, 86%, 87%, 88%, 89%, 90%, 91%, 92%, 93%, 94%, 95%, 96%, 97%, 98% or 99% sequence identity to SEQ ID NO: 253;
s) a variable heavy (VH) chain sequence comprising the amino acid sequence of SEQ ID NO: 254 or a sequence having at least 85%, 86%, 87%, 88%, 89%, 90%, 91%, 92%, 93%, 94%, 95%, 96%, 97%, 98% or 99% sequence identity to SEQ ID NO: 254; and a variable light (VL) chain sequence comprising the amino acid sequence of SEQ ID NO: 255 or a sequence having at least 85%, 86%, 87%, 88%, 89%, 90%, 91%, 92%, 93%, 94%, 95%, 96%, 97%, 98% or 99% sequence identity to SEQ ID NO: 255;
t) a variable heavy (VH) chain sequence comprising the amino acid sequence of SEQ ID NO: 256 or a sequence having at least 85%, 86%, 87%, 88%, 89%, 90%, 91%, 92%, 93%, 94%, 95%, 96%, 97%, 98% or 99% sequence identity to SEQ ID NO: 256; and a variable light (VL) chain sequence comprising the amino acid sequence of SEQ ID NO: 257 or a sequence having at least 85%, 86%, 87%, 88%, 89%, 90%, 91%, 92%, 93%, 94%, 95%, 96%, 97%, 98% or 99% sequence identity to SEQ ID NO: 257;
u) a variable heavy (VH) chain sequence comprising the amino acid sequence of SEQ ID NO: 258 or a sequence having at least 85%, 86%, 87%, 88%, 89%, 90%, 91%, 92%, 93%, 94%, 95%, 96%, 97%, 98% or 99% sequence identity to SEQ ID NO: 258; and a variable light (VL) chain sequence comprising the amino acid sequence of SEQ ID NO: 259 or a sequence having at least 85%, 86%, 87%, 88%, 89%, 90%, 91%, 92%, 93%, 94%, 95%, 96%, 97%, 98% or 99% sequence identity to SEQ ID NO: 259;
v) a variable heavy (VH) chain sequence comprising the amino acid sequence of SEQ ID NO: 260 or a sequence having at least 85%, 86%, 87%, 88%, 89%, 90%, 91%, 92%, 93%, 94%, 95%, 96%, 97%, 98% or 99% sequence identity to SEQ ID NO: 260; and a variable light (VL) chain sequence comprising the amino acid sequence of SEQ ID NO: 261 or a sequence having at least 85%, 86%, 87%, 88%, 89%, 90%, 91%, 92%, 93%, 94%, 95%, 96%, 97%, 98% or 99% sequence identity to SEQ ID NO: 261;
w) a variable heavy (VH) chain sequence comprising the amino acid sequence of SEQ ID NO: 262 or a sequence having at least 85%, 86%, 87%, 88%, 89%, 90%, 91%, 92%, 93%, 94%, 95%, 96%, 97%, 98% or 99% sequence identity to SEQ ID NO: 262; and a variable light (VL) chain sequence comprising the amino acid sequence of SEQ ID NO: 263 or a sequence having at least 85%, 86%, 87%, 88%, 89%, 90%, 91%, 92%, 93%, 94%, 95%, 96%, 97%, 98% or 99% sequence identity to SEQ ID NO: 263;
x) a variable heavy (VH) chain sequence comprising the amino acid sequence of SEQ ID NO: 264 or a sequence having at least 85%, 86%, 87%, 88%, 89%, 90%, 91%, 92%, 93%, 94%, 95%, 96%, 97%, 98% or 99% sequence identity to SEQ ID NO: 264; and a variable light (VL) chain sequence comprising the amino acid sequence of SEQ ID NO: 265 or a sequence having at least 85%, 86%, 87%, 88%, 89%, 90%, 91%, 92%, 93%, 94%, 95%, 96%, 97%, 98% or 99% sequence identity to SEQ ID NO: 265;
y) a variable heavy (VH) chain sequence comprising the amino acid sequence of SEQ ID NO: 266 or a sequence having at least 85%, 86%, 87%, 88%, 89%, 90%, 91%, 92%, 93%, 94%, 95%, 96%, 97%, 98% or 99% sequence identity to SEQ ID NO: 266; and a variable light (VL) chain sequence comprising the amino acid sequence of SEQ ID NO: 267 or a sequence having at least 85%, 86%, 87%, 88%, 89%, 90%, 91%, 92%, 93%, 94%, 95%, 96%, 97%, 98% or 99% sequence identity to SEQ ID NO: 267;
z) a variable heavy (VH) chain sequence comprising the amino acid sequence of SEQ ID NO: 268 or a sequence having at least 85%, 86%, 87%, 88%, 89%, 90%, 91%, 92%, 93%, 94%, 95%, 96%, 97%, 98% or 99% sequence identity to SEQ ID NO: 268; and a variable light (VL) chain sequence comprising the amino acid sequence of SEQ ID NO: 269 or a sequence having at least 85%, 86%, 87%, 88%, 89%, 90%, 91%, 92%, 93%, 94%, 95%, 96%, 97%, 98% or 99% sequence identity to SEQ ID NO: 269;
aa) a variable heavy (VH) chain sequence comprising the amino acid sequence of SEQ ID NO: 270 or a sequence having at least 85%, 86%, 87%, 88%, 89%, 90%, 91%, 92%, 93%, 94%, 95%, 96%, 97%, 98% or 99% sequence identity to SEQ ID NO: 270; and a variable light (VL) chain sequence comprising the amino acid sequence of SEQ ID NO: 271 or a sequence having at least 85%, 86%, 87%, 88%, 89%, 90%, 91%, 92%, 93%, 94%, 95%, 96%, 97%, 98% or 99% sequence identity to SEQ ID NO: 271;
ab) a variable heavy (VH) chain sequence comprising the amino acid sequence of SEQ ID NO: 272 or a sequence having at least 85%, 86%, 87%, 88%, 89%, 90%, 91%, 92%, 93%, 94%, 95%, 96%, 97%, 98% or 99% sequence identity to SEQ ID NO: 272; and a variable light (VL) chain sequence comprising the amino acid sequence of SEQ ID NO: 273 or a sequence having at least 85%, 86%, 87%, 88%, 89%, 90%, 91%, 92%, 93%, 94%, 95%, 96%, 97%, 98% or 99% sequence identity to SEQ ID NO: 273;
ac) a variable heavy (VH) chain sequence comprising the amino acid sequence of SEQ ID NO: 274 or a sequence having at least 85%, 86%, 87%, 88%, 89%, 90%, 91%, 92%, 93%, 94%, 95%, 96%, 97%, 98% or 99% sequence identity to SEQ ID NO: 274; and a variable light (VL) chain sequence comprising the amino acid sequence of SEQ ID NO: 275 or a sequence having at least 85%, 86%, 87%, 88%, 89%, 90%, 91%, 92%, 93%, 94%, 95%, 96%, 97%, 98% or 99% sequence identity to SEQ ID NO: 275;
ad) a variable heavy (VH) chain sequence comprising the amino acid sequence of SEQ ID NO: 276 or a sequence having at least 85%, 86%, 87%, 88%, 89%, 90%, 91%, 92%, 93%, 94%, 95%, 96%, 97%, 98% or 99% sequence identity to SEQ ID NO: 276; and a variable light (VL) chain sequence comprising the amino acid sequence of SEQ ID NO: 277 or a sequence having at least 85%, 86%, 87%, 88%, 89%, 90%, 91%, 92%, 93%, 94%, 95%, 96%, 97%, 98% or 99% sequence identity to SEQ ID NO: 277;
ae) a variable heavy (VH) chain sequence comprising the amino acid sequence of SEQ ID NO: 278 or a sequence having at least 85%, 86%, 87%, 88%, 89%, 90%, 91%, 92%, 93%, 94%, 95%, 96%, 97%, 98% or 99% sequence identity to SEQ ID NO: 278; and a variable light (VL) chain sequence comprising the amino acid sequence of SEQ ID NO: 279 or a sequence having at least 85%, 86%, 87%, 88%, 89%, 90%, 91%, 92%, 93%, 94%, 95%, 96%, 97%, 98% or 99% sequence identity to SEQ ID NO: 279;
af) a variable heavy (VH) chain sequence comprising the amino acid sequence of SEQ ID NO: 324 or a sequence having at least 85%, 86%, 87%, 88%, 89%, 90%, 91%, 92%, 93%, 94%, 95%, 96%, 97%, 98% or 99% sequence identity to SEQ ID NO: 324; and a variable light (VL) chain sequence comprising the amino acid sequence of SEQ ID NO: 328 or a sequence having at least 85%, 86%, 87%, 88%, 89%, 90%, 91%, 92%, 93%, 94%, 95%, 96%, 97%, 98% or 99% sequence identity to SEQ ID NO: 328;
ag) a variable heavy (VH) chain sequence comprising the amino acid sequence of SEQ ID NO: 325 or a sequence having at least 85%, 86%, 87%, 88%, 89%, 90%, 91%, 92%, 93%, 94%, 95%, 96%, 97%, 98% or 99% sequence identity to SEQ ID NO: 325; and a variable light (VL) chain sequence comprising the amino acid sequence of SEQ ID NO: 329 or a sequence having at least 85%, 86%, 87%, 88%, 89%, 90%, 91%, 92%, 93%, 94%, 95%, 96%, 97%, 98% or 99% sequence identity to SEQ ID NO: 329;
ah) a variable heavy (VH) chain sequence comprising the amino acid sequence of SEQ ID NO: 326 or a sequence having at least 85%, 86%, 87%, 88%, 89%, 90%, 91%, 92%, 93%, 94%, 95%, 96%, 97%, 98% or 99% sequence identity to SEQ ID NO: 326; and a variable light (VL) chain sequence comprising the amino acid sequence of SEQ ID NO: 330 or a sequence having at least 85%, 86%, 87%, 88%, 89%, 90%, 91%, 92%, 93%, 94%, 95%, 96%, 97%, 98% or 99% sequence identity to SEQ ID NO: 330;
ai) a variable heavy (VH) chain sequence comprising the amino acid sequence of SEQ ID NO: 327 or a sequence having at least 85%, 86%, 87%, 88%, 89%, 90%, 91%, 92%, 93%, 94%, 95%, 96%, 97%, 98% or 99% sequence identity to SEQ ID NO: 327; and a variable light (VL) chain sequence comprising the amino acid sequence of SEQ ID NO: 331 or a sequence having at least 85%, 86%, 87%, 88%, 89%, 90%, 91%, 92%, 93%, 94%, 95%, 96%, 97%, 98% or 99% sequence identity to SEQ ID NO: 331;
aj) a variable heavy (VH) chain sequence comprising the amino acid sequence of SEQ ID NO: 382 or a sequence having at least 85%, 86%, 87%, 88%, 89%, 90%, 91%, 92%, 93%, 94%, 95%, 96%, 97%, 98% or 99% sequence identity to SEQ ID NO: 382; and a variable light (VL) chain sequence comprising the amino acid sequence of SEQ ID NO: 392 or a sequence having at least 85%, 86%, 87%, 88%, 89%, 90%, 91%, 92%, 93%, 94%, 95%, 96%, 97%, 98% or 99% sequence identity to SEQ ID NO: 392;
ak) a variable heavy (VH) chain sequence comprising the amino acid sequence of SEQ ID NO: 383 or a sequence having at least 85%, 86%, 87%, 88%, 89%, 90%, 91%, 92%, 93%, 94%, 95%, 96%, 97%, 98% or 99% sequence identity to SEQ ID NO: 383; and a variable light (VL) chain sequence comprising the amino acid sequence of SEQ ID NO: 393 or a sequence having at least 85%, 86%, 87%, 88%, 89%, 90%, 91%, 92%, 93%, 94%, 95%, 96%, 97%, 98% or 99% sequence identity to SEQ ID NO: 393;
al) a variable heavy (VH) chain sequence comprising the amino acid sequence of SEQ ID NO: 384 or a sequence having at least 85%, 86%, 87%, 88%, 89%, 90%, 91%, 92%, 93%, 94%, 95%, 96%, 97%, 98% or 99% sequence identity to SEQ ID NO: 384; and a variable light (VL) chain sequence comprising the amino acid sequence of SEQ ID NO: 394 or a sequence having at least 85%, 86%, 87%, 88%, 89%, 90%, 91%, 92%, 93%, 94%, 95%, 96%, 97%, 98% or 99% sequence identity to SEQ ID NO: 394;
am) a variable heavy (VH) chain sequence comprising the amino acid sequence of SEQ ID NO: 385 or a sequence having at least 85%, 86%, 87%, 88%, 89%, 90%, 91%, 92%, 93%, 94%, 95%, 96%, 97%, 98% or 99% sequence identity to SEQ ID NO: 385; and a variable light (VL) chain sequence comprising the amino acid sequence of SEQ ID NO: 395 or a sequence having at least 85%, 86%, 87%, 88%, 89%, 90%, 91%, 92%, 93%, 94%, 95%, 96%, 97%, 98% or 99% sequence identity to SEQ ID NO: 395;
an) a variable heavy (VH) chain sequence comprising the amino acid sequence of SEQ ID NO: 386 or a sequence having at least 85%, 86%, 87%, 88%, 89%, 90%, 91%, 92%, 93%, 94%, 95%, 96%, 97%, 98% or 99% sequence identity to SEQ ID NO: 386; and a variable light (VL) chain sequence comprising the amino acid sequence of SEQ ID NO: 396 or a sequence having at least 85%, 86%, 87%, 88%, 89%, 90%, 91%, 92%, 93%, 94%, 95%, 96%, 97%, 98% or 99% sequence identity to SEQ ID NO: 396;
ao) a variable heavy (VH) chain sequence comprising the amino acid sequence of SEQ ID NO: 387 or a sequence having at least 85%, 86%, 87%, 88%, 89%, 90%, 91%, 92%, 93%, 94%, 95%, 96%, 97%, 98% or 99% sequence identity to SEQ ID NO: 387; and a variable light (VL) chain sequence comprising the amino acid sequence of SEQ ID NO: 397 or a sequence having at least 85%, 86%, 87%, 88%, 89%, 90%, 91%, 92%, 93%, 94%, 95%, 96%, 97%, 98% or 99% sequence identity to SEQ ID NO: 397;
ap) a variable heavy (VH) chain sequence comprising the amino acid sequence of SEQ ID NO: 388 or a sequence having at least 85%, 86%, 87%, 88%, 89%, 90%, 91%, 92%, 93%, 94%, 95%, 96%, 97%, 98% or 99% sequence identity to SEQ ID NO: 388; and a variable light (VL) chain sequence comprising the amino acid sequence of SEQ ID NO: 398 or a sequence having at least 85%, 86%, 87%, 88%, 89%, 90%, 91%, 92%, 93%, 94%, 95%, 96%, 97%, 98% or 99% sequence identity to SEQ ID NO: 398;
aq) a variable heavy (VH) chain sequence comprising the amino acid sequence of SEQ ID NO: 389 or a sequence having at least 85%, 86%, 87%, 88%, 89%, 90%, 91%, 92%, 93%, 94%, 95%, 96%, 97%, 98% or 99% sequence identity to SEQ ID NO: 389; and a variable light (VL) chain sequence comprising the amino acid sequence of SEQ ID NO: 399 or a sequence having at least 85%, 86%, 87%, 88%, 89%, 90%, 91%, 92%, 93%, 94%, 95%, 96%, 97%, 98% or 99% sequence identity to SEQ ID NO: 399;
ar) a variable heavy (VH) chain sequence comprising the amino acid sequence of SEQ ID NO: 390 or a sequence having at least 85%, 86%, 87%, 88%, 89%, 90%, 91%, 92%, 93%, 94%, 95%, 96%, 97%, 98% or 99% sequence identity to SEQ ID NO: 390; and a variable light (VL) chain sequence comprising the amino acid sequence of SEQ ID NO: 400 or a sequence having at least 85%, 86%, 87%, 88%, 89%, 90%, 91%, 92%, 93%, 94%, 95%, 96%, 97%, 98% or 99% sequence identity to SEQ ID NO: 400;
as) a variable heavy (VH) chain sequence comprising the amino acid sequence of SEQ ID NO: 391 or a sequence having at least 85%, 86%, 87%, 88%, 89%, 90%, 91%, 92%, 93%, 94%, 95%, 96%, 97%, 98% or 99% sequence identity to SEQ ID NO: 391; and a variable light (VL) chain sequence comprising the amino acid sequence of SEQ ID NO: 401 or a sequence having at least 85%, 86%, 87%, 88%, 89%, 90%, 91%, 92%, 93%, 94%, 95%, 96%, 97%, 98% or 99% sequence identity to SEQ ID NO: 401;
at) a variable heavy (VH) chain sequence comprising the amino acid sequence of SEQ ID NO: 452 or a sequence having at least 85%, 86%, 87%, 88%, 89%, 90%, 91%, 92%, 93%, 94%, 95%, 96%, 97%, 98% or 99% sequence identity to SEQ ID NO: 452; and a variable light (VL) chain sequence comprising the amino acid sequence of SEQ ID NO: 462 or a sequence having at least 85%, 86%, 87%, 88%, 89%, 90%, 91%, 92%, 93%, 94%, 95%, 96%, 97%, 98% or 99% sequence identity to SEQ ID NO: 462;
au) a variable heavy (VH) chain sequence comprising the amino acid sequence of SEQ ID NO: 453 or a sequence having at least 85%, 86%, 87%, 88%, 89%, 90%, 91%, 92%, 93%, 94%, 95%, 96%, 97%, 98% or 99% sequence identity to SEQ ID NO: 453; and a variable light (VL) chain sequence comprising the amino acid sequence of SEQ ID NO: 463 or a sequence having at least 85%, 86%, 87%, 88%, 89%, 90%, 91%, 92%, 93%, 94%, 95%, 96%, 97%, 98% or 99% sequence identity to SEQ ID NO: 463;
av) a variable heavy (VH) chain sequence comprising the amino acid sequence of SEQ ID NO: 454 or a sequence having at least 85%, 86%, 87%, 88%, 89%, 90%, 91%, 92%, 93%, 94%, 95%, 96%, 97%, 98% or 99% sequence identity to SEQ ID NO: 454; and a variable light (VL) chain sequence comprising the amino acid sequence of SEQ ID NO: 464 or a sequence having at least 85%, 86%, 87%, 88%, 89%, 90%, 91%, 92%, 93%, 94%, 95%, 96%, 97%, 98% or 99% sequence identity to SEQ ID NO: 464;
aw) a variable heavy (VH) chain sequence comprising the amino acid sequence of SEQ ID NO: 455 or a sequence having at least 85%, 86%, 87%, 88%, 89%, 90%, 91%, 92%, 93%, 94%, 95%, 96%, 97%, 98% or 99% sequence identity to SEQ ID NO: 455; and a variable light (VL) chain sequence comprising the amino acid sequence of SEQ ID NO: 465 or a sequence having at least 85%, 86%, 87%, 88%, 89%, 90%, 91%, 92%, 93%, 94%, 95%, 96%, 97%, 98% or 99% sequence identity to SEQ ID NO: 465;
ax) a variable heavy (VH) chain sequence comprising the amino acid sequence of SEQ ID NO: 456 or a sequence having at least 85%, 86%, 87%, 88%, 89%, 90%, 91%, 92%, 93%, 94%, 95%, 96%, 97%, 98% or 99% sequence identity to SEQ ID NO: 456; and a variable light (VL) chain sequence comprising the amino acid sequence of SEQ ID NO: 466 or a sequence having at least 85%, 86%, 87%, 88%, 89%, 90%, 91%, 92%, 93%, 94%, 95%, 96%, 97%, 98% or 99% sequence identity to SEQ ID NO: 466;
ay) a variable heavy (VH) chain sequence comprising the amino acid sequence of SEQ ID NO: 457 or a sequence having at least 85%, 86%, 87%, 88%, 89%, 90%, 91%, 92%, 93%, 94%, 95%, 96%, 97%, 98% or 99% sequence identity to SEQ ID NO: 457; and a variable light (VL) chain sequence comprising the amino acid sequence of SEQ ID NO: 467 or a sequence having at least 85%, 86%, 87%, 88%, 89%, 90%, 91%, 92%, 93%, 94%, 95%, 96%, 97%, 98% or 99% sequence identity to SEQ ID NO: 467;
az) a variable heavy (VH) chain sequence comprising the amino acid sequence of SEQ ID NO: 458 or a sequence having at least 85%, 86%, 87%, 88%, 89%, 90%, 91%, 92%, 93%, 94%, 95%, 96%, 97%, 98% or 99% sequence identity to SEQ ID NO: 458; and a variable light (VL) chain sequence comprising the amino acid sequence of SEQ ID NO: 468 or a sequence having at least 85%, 86%, 87%, 88%, 89%, 90%, 91%, 92%, 93%, 94%, 95%, 96%, 97%, 98% or 99% sequence identity to SEQ ID NO: 468;
ba) a variable heavy (VH) chain sequence comprising the amino acid sequence of SEQ ID NO: 459 or a sequence having at least 85%, 86%, 87%, 88%, 89%, 90%, 91%, 92%, 93%, 94%, 95%, 96%, 97%, 98% or 99% sequence identity to SEQ ID NO: 459; and a variable light (VL) chain sequence comprising the amino acid sequence of SEQ ID NO: 469 or a sequence having at least 85%, 86%, 87%, 88%, 89%, 90%, 91%, 92%, 93%, 94%, 95%, 96%, 97%, 98% or 99% sequence identity to SEQ ID NO: 469;
bb) a variable heavy (VH) chain sequence comprising the amino acid sequence of SEQ ID NO: 460 or a sequence having at least 85%, 86%, 87%, 88%, 89%, 90%, 91%, 92%, 93%, 94%, 95%, 96%, 97%, 98% or 99% sequence identity to SEQ ID NO: 460; and a variable light (VL) chain sequence comprising the amino acid sequence of SEQ ID NO: 470 or a sequence having at least 85%, 86%, 87%, 88%, 89%, 90%, 91%, 92%, 93%, 94%, 95%, 96%, 97%, 98% or 99% sequence identity to SEQ ID NO: 470; or
bc) a variable heavy (VH) chain sequence comprising the amino acid sequence of SEQ ID NO: 461 or a sequence having at least 85%, 86%, 87%, 88%, 89%, 90%, 91%, 92%, 93%, 94%, 95%, 96%, 97%, 98% or 99% sequence identity to SEQ ID NO: 461; and a variable light (VL) chain sequence comprising the amino acid sequence of SEQ ID NO: 471 or a sequence having at least 85%, 86%, 87%, 88%, 89%, 90%, 91%, 92%, 93%, 94%, 95%, 96%, 97%, 98% or 99% sequence identity to SEQ ID NO: 471.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Conservative substitutions are substitutions that do not, or not substantially impair the primary properties (e.g. binding affinity or coronavirus replication reduction capacity) of the antibody or antigen-binding fragment thereof. In some embodiments, the conservative substitutions include at least one substitution selected from the group consisting of: Ala (A)-Val; Arg (R)-Lys; Asn (N)-Gin; Asp (D)-Glu; Cys (C)-Ser; Gln (Q)-Asn; Glu (E)-Asp; Gly (G)-Ala; His (H)-Arg; Ile (I)-Leu; Leu (L)-Ile; Lys (K)-Arg; Met (M)-Leu; Phe (F)-Tyr; Pro (P)-Ala; Ser(S)-Thr; Thr (T)-Ser; Trp (W)-Tyr; Tyr (Y)-Phe; and Val (V)-Leu. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. In certain embodiments, substitutions, insertions, or deletions occur in regions outside the CDRs (i.e., in the FRs).

In certain embodiments, amino acid sequence variants of the antibodies provided herein are contemplated. For example, it may be desirable to improve the binding affinity to the coronavirus S protein and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding or coronavirus replication reduction capacity.

In certain embodiments, antibody variants having one or more amino acid substitutions are provided. Amino acid substitutions may be introduced into an antibody of interest and the products screened for a desired activity, e.g., retained/improved antigen binding, decreased immunogenicity, half-life, or altered ADCC or CDC.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, increased coronavirus replication reduction capacity, reduced immunogenicity) relative to the parent antibody and/or will have substantially retained certain biological properties of the parent antibody. An exemplary substitutional variant is an affinity-matured antibody, which may be conveniently generated. Briefly, one or more CDR residues are mutated and the variant antibodies displayed on phage and screened for a particular biological activity (e.g. binding affinity or coronavirus replication reduction capacity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, 2008, Methods Mol. Biol. 207:179-196), and/or SDRs (a-CDRs), with the resulting variant VH or VL being tested for binding affinity. Affinity maturation by constructing and reselecting from secondary libraries has been described, e.g., in Hoogenboom et al., 2002 in Methods in Molecular Biology 178:1-37. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted. In another embodiment look-through mutagenesis is used to optimize antibody affinity with a multidimensional mutagenesis method that simultaneously assesses and optimizes combinatorial mutations of selected amino acids (Rajpal, Arvind et al., 2005, Proceedings of the National Academy of Sciences of the United States of America vol. 102,24:8466-71).

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity and/or coronavirus replication reduction capacity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells, 1989, Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., 1997, TIBTECH 15:26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g., complex, hybrid and high mannose structures) as measured by MALDI-TOF mass spectrometry, as described in WO 2008/077546, for example. Asn297 refers to the asparagine residue located at about position 297 in the Fc region (Eu numbering of Fe region residues); however, Asn297 may also be located about +3 amino acids upstream or downstream of position 297, i.e., between positions 294 and 300, due to minor sequence variations in antibodies. Such fucosylation variants may have an altered influence on inflammation (Irvine, Edward B, and Galit Alter., 2020, Glycobiology vol. 30,4:241-253). See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki et al. 2004 J. Mol. Biol. 336:1239-1249; Yamane-Ohnuki et al., 2004, Biotech. Bioeng. 87:614. Examples of cell lines capable of producing defucosylated antibodies include Lec13 CHO cells deficient in protein fucosylation (Ripka et al., 1986, Arch. Biochem. Biophys. 249:533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al., 2004, Biotech. Bioeng. 87:614; Kanda, Y. et al., 2006, Biotechnol. Bioeng., 94 (4): 680-688; and WO 2003/085107).

Antibodies variants are further provided with bisected oligosaccharides, e.g., in which a biantennary oligosaccharide attached to the Fc region of the antibody is bisected by GlcNAc. Such antibody variants may have altered fucosylation and/or altered influence on inflammation (Irvine, Edward B, and Galit Alter., 2020, Glycobiology vol. 30,4:241-253). Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

Antibodies with increased half-lives and improved binding to the neonatal Fc receptor (FcRn), which is responsible for the transfer of maternal IgGs to the fetus (Guyer et al., 1976, J. Immunol. 117:587 and Kirn et al., 1994 J. Immunol. 24:249), are described in US2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (US 2006/0194291).

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

In certain embodiments, an antibody provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, the invention relates to an antibody, or antigen-binding fragment thereof, comprising at least one of the sequences described above, wherein the antibody is an IgM, IgG1, IgG2a or IgG2b, IgG3, IgG4, IgA or IgE antibody.

In certain embodiments, the invention relates to an antibody, or antigen-binding fragment thereof, comprising at least one of the sequences described above, wherein the antigen-binding fragment is a Fab fragment, an F(ab′) fragment or an Fv fragment.

In any of the embodiments described herein, the antibody may be a monoclonal antibody. In any of the embodiments described herein, the antibody may be human, humanized, or chimeric antibody. There are five major classes of antibodies: IgA, IgD, IgE, IgG, and IgM, and several of these may be further divided into subclasses, e.g., IgG1, IgG2, IgG3, IgG4, IgA1, and IgA2. The heavy chain constant domains that correspond to the different classes of immunoglobulins are called α, δ, ε, γ, and μ, respectively. In any of the embodiments described herein, the antibody may be an IgG1, IgG2a or IgG2b, IgG3, IgG4, IgM, IgA (e.g., IgA1, IgA2), IgAsec, IgD, IgE. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, an F(ab′) fragment, an Fv fragment, an F(ab′) 2 fragment and/or a single-chain Fv fragment.

A “Fab fragment” as used herein is comprised of one light chain and the CH1 and variable regions of one heavy chain. The heavy chain of a Fab molecule cannot form a disulfide bond with another heavy chain molecule.

A “F(ab′) fragment” contains one light chain and a portion of one heavy chain that contains the VH domain and the C H1 domain and also the region between the CH1 and C H2 domains, such that an interchain disulfide bond can be formed between the two heavy chains of two F (ab′) fragments to form a F (ab′) 2 molecule.

A “Fv fragment” comprises the variable regions from both the heavy and light chains, but lacks the constant regions.

A “F(ab′) 2 fragment” contains two light chains and two heavy chains containing a portion of the constant region between the CH1 and CH2 domains, such that an interchain disulfide bond is formed between the two heavy chains. A F (ab′) 2 fragment thus is composed of two Fab′ fragments that are held together by a disulfide bond between the two heavy chains.

“Single-chain Fv” or “scFv” antibody fragments have, in the context of the invention, the VH and VL domains of an antibody, wherein these domains are present in a single polypeptide chain. Generally, the scFv polypeptide further comprises a polypeptide linker between the VH and VL domains which enables the scFv to form the desired structure for antigen binding. Techniques described for the production of single chain antibodies are described, e.g., in Pluckthun, A., 1994, The Pharmacology of Monoclonal Antibodies, 269-315.

An “Fc region” contains two heavy chain fragments comprising the CH2 and CH3 domains of an antibody. The two heavy chain fragments are held together by two or more disulfide bonds and by hydrophobic interactions of the CH3 domains.

Antibodies, antibody constructs, antibody fragments, antibody derivatives (all being Ig-derived) to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook et al., 1989, Molecular Cloning: A Laboratory Manual; Cold Spring Harbor Laboratory Press. The term “lg-derived domain” particularly relates to (poly) peptide constructs comprising at least one CDR. Fragments or derivatives of the recited Ig-derived domains define (poly) peptides which are parts of the above antibody molecules and/or which are modified by chemical/biochemical or molecular biological methods. Corresponding methods are known in the art and described inter alia in laboratory manuals (see Sambrook et al., Molecular Cloning: A Laboratory Manual; Cold Spring Harbor Laboratory Press, 2nd edition (1989) and 3rd edition (2001); Gerhardt et al., 1994, Methods for General and Molecular Bacteriology ASM Press; Lefkovits, 1997, Immunology Methods Manual: The Comprehensive Sourcebook of Techniques; Academic Press); Golemis, 2002, Protein-Protein Interactions: A Molecular Cloning Manual Cold Spring Harbor Laboratory Press).

In certain embodiments the antibody, or antigen binding fragment thereof, of the invention is of a certain class or a certain fragment described above to enable a certain distribution in the body. For example, the antibody, or antigen binding fragment thereof, of the invention may be of a certain class (e.g. IgM, IgA, IgAsec, IgD, IgE), or a fragment as described above in order to avoid active placenta transfer and/or accumulation in the fetus during pregnancy (e.g. as described for IgG antibodies, e.g., in Palmeira, Patricia, et al. 2012 Clinical and Developmental Immunology).

Accordingly, in the context of the present invention, the antibody molecule described herein above is selected from the group consisting of a full antibody (immunoglobulin, like an IgG1, an IgG2, an IgG2a, an IgG2b, an IgA1, an IgGA2, an IgG3, an IgG4, an IgA, an IgM, an IgD or an IgE), F (ab)-, Fab′-SH-, Fv-, Fab′-, F (ab′) 2-fragment, a chimeric antibody, a CDR-grafted antibody, a fully human antibody, a bivalent antibody-construct, an antibody-fusion protein, a synthetic antibody, bivalent single chain antibody, a trivalent single chain antibody and a multivalent single chain antibody.

Accordingly, the invention is at least in part based on the finding that the antibodies or fragments thereof, described herein are more resistant to coronavirus mutations.

In certain embodiments, the invention relates to the antibody, or antigen-binding fragment thereof, of the invention, the antibody, or antigen-binding fragment thereof, comprising a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51.

In certain embodiments, the invention relates to the antibody, or antigen-binding fragment thereof, of the invention, the antibody, or antigen-binding fragment thereof, comprising a variable heavy (VH) chain sequence comprising the amino acid sequence of SEQ ID NO: 230 or a sequence having at least 85%, 86%, 87%, 88%, 89%, 90%, 91%, 92%, 93%, 94%, 95%, 96%, 97%, 98% or 99% sequence identity to SEQ ID NO: 230; and a variable light (VL) chain sequence comprising the amino acid sequence of SEQ ID NO: 231 or a sequence having at least 85%, 86%, 87%, 88%, 89%, 90%, 91%, 92%, 93%, 94%, 95%, 96%, 97%, 98% or 99% sequence identity to SEQ ID NO: 231.

Fp.007 is a neutralizing antibody comprising the CDRs described above and recognizes FP (SEQ ID NO: 1) but displays an unexpected pattern of cross-reactivity (FIG. 1k). Fp.007 strongly bound S even in the absence of ACE2, indicating that optimal FP recognition by neutralizing antibodies does not always require ACE2-induced conformational changes (FIG. 2h).

In certain embodiments, the invention relates to the antibody, or antigen-binding fragment thereof, of the invention, the antibody, or antigen-binding fragment thereof, comprising a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167.

In certain embodiments, the invention relates to the antibody, or antigen-binding fragment thereof, of the invention, the antibody, or antigen-binding fragment thereof, comprising a variable heavy (VH) chain sequence comprising the amino acid sequence of SEQ ID NO: 276 or a sequence having at least 85%, 86%, 87%, 88%, 89%, 90%, 91%, 92%, 93%, 94%, 95%, 96%, 97%, 98% or 99% sequence identity to SEQ ID NO: 276; and a variable light (VL) chain sequence comprising the amino acid sequence of SEQ ID NO: 277 or a sequence having at least 85%, 86%, 87%, 88%, 89%, 90%, 91%, 92%, 93%, 94%, 95%, 96%, 97%, 98% or 99% sequence identity to SEQ ID NO: 277;

The inventors found that antibody sd1.040 comprising the sequences described above does not neutralize by interfering with ACE2 receptor binding. Instead, without being bound by theory its function is likely linked to conformational selection and/or stabilization of specific S conformers.

This contrasts murine antibody S3H3 that recognizes trimeric S and potentially functions by “locking” the release of S1 subunits from S2. Although not the most potent antibody by itself, synergistic activity of sd1.040 with other antibodies protects animals from viral challenge.

Accordingly, the invention is at least in part based on the finding that sd1.040 can enhance the effect of other (endogenous or recombinant) antibodies as described herein.

In certain embodiments, the invention relates to the antibody, or antigen-binding fragment thereof, of the invention, the antibody, or antigen-binding fragment thereof, comprising a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46.

Fp.006, comprising the sequences mentioned above is an unexpectantly potent antibody. Accordingly, the invention is at least in part based on the potency of the Fp.006 antibody.

In certain embodiments, the invention relates to the antibody, or antigen-binding fragment thereof, of the invention, the antibody, or antigen-binding fragment thereof, comprising a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96.

In certain embodiments, the invention relates to the antibody, or antigen-binding fragment thereof, of the invention, the antibody, or antigen-binding fragment thereof, comprising a variable heavy (VH) chain sequence comprising the amino acid sequence of SEQ ID NO: 248 or a sequence having at least 85%, 86%, 87%, 88%, 89%, 90%, 91%, 92%, 93%, 94%, 95%, 96%, 97%, 98% or 99% sequence identity to SEQ ID NO: 248; and a variable light (VL) chain sequence comprising the amino acid sequence of SEQ ID NO: 249 or a sequence having at least 85%, 86%, 87%, 88%, 89%, 90%, 91%, 92%, 93%, 94%, 95%, 96%, 97%, 98% or 99% sequence identity to SEQ ID NO: 249.

In certain embodiments, the invention relates to the antibody, or antigen-binding fragment thereof, of the invention, the antibody, or antigen-binding fragment thereof, comprising v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126.

In certain embodiments, the invention relates to the antibody, or antigen-binding fragment thereof, of the invention, the antibody, or antigen-binding fragment thereof, comprising a variable heavy (VH) chain sequence comprising the amino acid sequence of SEQ ID NO: 260 or a sequence having at least 85%, 86%, 87%, 88%, 89%, 90%, 91%, 92%, 93%, 94%, 95%, 96%, 97%, 98% or 99% sequence identity to SEQ ID NO: 260; and a variable light (VL) chain sequence comprising the amino acid sequence of SEQ ID NO: 261 or a sequence having at least 85%, 86%, 87%, 88%, 89%, 90%, 91%, 92%, 93%, 94%, 95%, 96%, 97%, 98% or 99% sequence identity to SEQ ID NO: 261.

ACE2 binding to cell surface-expressed S increased the attachment of hr2.016 and hr2.023 to S (2.3- and 2.4-fold, respectively; FIG. 2i). Thus, conformational changes of S induced by ACE2 expose highly conserved neutralizing epitopes both at FP and HR2 regions.

Accordingly, the invention is at least in part based on the selective binding of hr2.016 and hr2.023.

In certain embodiments, the invention relates to the antibody, or antigen-binding fragment thereof, of the invention, wherein the antibody, or antigen-binding fragment thereof, is cross-specific with the S proteins of SARS-COV-2, MERS and HCOV-229E.

Cross-specificity of an antibody, or antigen-binding fragment thereof, may be tested, for example, by assessing binding of the antibody or antigen-binding fragments thereof, under conventional conditions (see, e.g., Harlow and Lane, 1988 Antibodies: A Laboratory Manual, Cold Spring Harbor Laboratory Press, and Harlow and Lane, 1999 using Antibodies: A Laboratory Manual, Cold Spring Harbor Laboratory Press) and/or according to the methods described in the examples. These methods may comprise, inter alia, binding studies, blocking and competition studies with structurally and/or functionally closely related molecules. These binding studies also comprise FACS analysis, surface plasmon resonance, analytical ultracentrifugation, isothermal titration calorimetry, fluorescence anisotropy, fluorescence spectroscopy or by radiolabeled ligand binding assays. Cross-specificity can be determined experimentally by methods known in the art and methods as described herein. Such methods comprise, but are not limited to Western Blots, ELISA-, RIA-, ECL-, IRMA-tests and peptide scans.

In some embodiments, the cross-specificity described herein relates to binding cross-specificity and/or cross-specificity in inhibiting biologic activity such as inhibiting reproducibility.

Accordingly, the invention is at least in part based on the cross-specificity of the antibodies, or antigen-binding fragments described herein.

In certain embodiments, the invention relates to the antibody, or antigen-binding fragment thereof, of the invention, wherein the conserved region is SEQ ID NO: 01 and wherein the antibody, or antigen-binding fragment thereof, is cross-specific for coronaviruses of the alpha, beta, gamma and delta genera.

The inventors identified a panel of anti-FP neutralizing antibodies that broadly recognize more distant coronaviruses, including all 9 of the known human CoVs.

Interestingly, without being bound by theory, for FP-specific antibodies, the inventors observed specific contacts with residue R815, in the S2′ cleavage site.

Accordingly, the invention is at least in part based on the cross-specificity of the antibodies, or antigen-binding fragments described herein.

In certain embodiments, the invention relates to the antibody, or antigen-binding fragment thereof, of the invention, wherein the conserved region is SEQ ID NO: 02 and wherein the antibody, or antigen-binding fragment thereof, is cross-specific for coronaviruses of the alpha, beta and gamma genera.

In certain embodiments, the invention relates to the antibody, or antigen-binding fragment thereof, of the invention, wherein the conserved region is SEQ ID NO: 02 and wherein the antibody, or antigen-binding fragment thereof, is cross-specific for coronaviruses of the beta and gamma genera.

In certain embodiments, the invention relates to the antibody, or antigen-binding fragment thereof, of the invention, wherein the conserved region is SEQ ID NO: 02 and wherein the antibody, or antigen-binding fragment thereof, is cross-specific for coronaviruses of the alpha and gamma genera.

In certain embodiments, the invention relates to the antibody, or antigen-binding fragment thereof, of the invention, wherein the conserved region is SEQ ID NO: 02 and wherein the antibody, or antigen-binding fragment thereof, is cross-specific for coronaviruses of the alpha and beta.

He inventors discovered that antibodies binding to the stem helix that precedes the HR2 region (e.g. hr2.016), can at the same time be potent (IC50 of 10 ng/ml) and broadly cross-specific with beta- and also with some alpha- and gammacoronaviruses.

Accordingly, the invention is at least in part based on the cross-specificity of the antibodies, or antigen-binding fragments described herein.

In certain embodiments, the invention relates to the antibody, or antigen-binding fragment thereof, of the invention, wherein the antibody, or antigen-binding fragment thereof, is cross-specific for at least 2, at least 3, at least 4, at least 5, at least 6 SARS-CoV-2 variants, preferably wherein the SARS-COV-2 variants are selected from the group of Lineage B.1.1.207, Lineage B.1.1.7, Cluster 5, 501.V2 variant, Lineage P.1, Lineage B.1.429/CAL.20C, Lineage B.1.525, Lineage B1.620, Lineage C 37, Lineage B.1.621, Lineage B.1.1.529 and Sublineages BA.1, BA.1.1, and BA.2.

In certain embodiments, the invention relates to the antibody, or antigen-binding fragment thereof, of the invention, wherein a) the conserved region is SEQ ID NO: 16 and/or SEQ ID NO: 298; and b) wherein the antibody, or antigen-binding fragment thereof, is cross-specific for SARS-COV-2 variants.

In some embodiments, the SARS-COV-2 variant described herein is at least one SARS-COV-2 variant comprising at least one mutation selected from the group consisting of: del 69-70, RSYLTPGD246-253N, N440K, G446V, L452R, Y453F, S477G/N, E484Q, E484K, F490S, N501Y, N501S, D614G, Q677P/H, P681H, P681R and A701V.

In some embodiments, the SARS-COV-2 variant described herein is at least one SARS-COV-2 variant selected from the group of Lineage B.1.1.207, Lineage B.1.1.7, Cluster 5, 501.V2 variant, Lineage P.1, Lineage B.1.429/CAL.20C, Lineage B.1.525, Lineage B1.620, Lineage C 37, Lineage B.1.621, Lineage B.1.617.2, Lineage B.1.1.529, Sublineage BA.1, Sublineage BA.1.1, Sublineage BA.2, Sublineage BA.2.12.1, Sublineage BA.2.9.1, Sublineage BA.2.11, Sublineage BA.2.13, Sublineage BA.4 and Sublineage BA.5.

In some embodiments, the SARS-COV-2 variant described herein is at least one SARS-COV-2 variant described by a Nextstrain clade selected from the group 19A, 20A, 20C, 20G, 20H, 20B, 20D, 20F, 20I, 20E, 21A, 21K and 22 (A, B, C).

In some aspects, the SARS-COV-2 virus described herein is a SARS-COV-2 variant having an at least 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% sequence identity to the viral genome sequence of at last one SARS-COV-2 variant described herein.

In certain embodiments, the invention relates to the antibody, or antigen-binding fragment thereof, of the invention, wherein the conserved region is a) SEQ ID NO: 16 and/or SEQ ID NO: 298; and b) wherein the antibody, or antigen-binding fragment thereof, is cross-specific for the SARS-COV-2 variants Lineage B.1.617.2, Lineage B.1.1.529, and Sublineages BA.1, BA.1.1, and BA.2.

Accordingly, the invention is at least in part based on the cross-specificity of the antibodies, or antigen-binding fragments described herein.

In certain embodiments, the invention relates to a multispecific antibody or a multispecific antigen-binding fragment thereof, specifically binding to at least two regions of a coronavirus S protein, wherein at least one of the coronavirus S protein is a conserved region, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298

The term “multispecific antibody” as used herein, refers to an antibody that binds to two or more different epitopes on at least two or more different targets (e.g., SEQ ID NO: 16/SEQ ID NO: 298 and RBD). The term “multispecific antibody” includes bispecific, trispecific, tetraspecific, pentaspecific and hexaspecific antibodies.

The inventors found that a multispecific e.g. a bispecific antibody binding to at least one of the conserved regions described herein can be more effective than the sum of the inhibition of two separate targets.

Accordingly, the invention is at least in part based on the synergizing effect of simultaneously binding to conserved region and a second region of a coronavirus S protein.

In some embodiments, the multispecific antibody described herein is specifically binding to a second region of a coronavirus S protein, wherein the second region is a non-conserved region.

In certain embodiments, the invention relates to the multispecific antibody or a multispecific antigen-binding fragment thereof, according to the invention, wherein the at least two regions of the coronavirus S protein comprise an RBD region of a coronavirus.

The inventors found that a multispecific, e.g., a bispecific antibody binding to at least one of the conserved regions described herein and the RBD region can be more effective than the sum of the inhibition of two separate targets.

Accordingly, the invention is at least in part based on the synergizing effect of simultaneously binding to a conserved region and the RBD region of a coronavirus S protein.

In some embodiments, the multispecific antibody described herein is specifically binding to a sequence as defined by SEQ ID NO: 16 and/or SEQ ID NO: 298 and to an RBD region such as a sequence as defined by SEQ ID NO: 301.

In certain embodiments, the invention relates to the multispecific antibody or the multispecific antigen-binding fragment thereof, of the invention, comprising at least one selected from the group of:
a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 comprising or consisting of the sequence of 161 and CDR3 as defined in SEQ ID NO: 162;
ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; and
bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451.

In certain embodiments, the invention relates to the multispecific antibody or the multispecific antigen-binding fragment thereof, of the invention, comprising a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167.

Accordingly, the invention is at least in part based on the synergizing effect of simultaneously binding to conserved region and a second region of a coronavirus S protein.

In certain embodiments, the invention relates to a polynucleotide encoding an antibody, or an antigen-binding fragment thereof, according to the invention.

The term “polynucleotide”, as used herein, refers to a nucleic acid sequence. The nucleic acid sequence may be a DNA or a RNA sequence, preferably the nucleic acid sequence is a DNA sequence. The polynucleotides of the present invention either essentially consist of the aforementioned nucleic acid sequences or comprise the aforementioned nucleic acid sequences. Thus, they may contain further nucleic acid sequences as well. The polynucleotides of the present invention shall be provided, preferably, either as an isolated polynucleotide (i.e. isolated from its natural context) or in genetically modified form. An isolated polynucleotide as referred to herein also encompasses polynucleotides which are present in cellular context other than their natural cellular context, i.e. heterologous polynucleotides. The term polynucleotide encompasses single as well as double stranded polynucleotides. Moreover, comprised are also chemically modified polynucleotides including naturally occurring modified polynucleotides such as glycosylated or methylated polynucleotides or artificial modified one such as biotinylated polynucleotides.

In an embodiment, the polynucleotide of the invention encodes at least one of a variable heavy (VH) chain sequence and/or a variable light (VL) chain sequence of an antibody described herein.

In certain embodiments the polynucleotide encoding the antibody, or the antigen-binding fragment thereof, of the invention is suitable for the use as a vector.

In certain embodiments the polynucleotide encoding the antibody, or the antigen-binding fragment thereof, of the invention is suitable for the use as a vector for transient transfection.

In certain embodiments the polynucleotide encoding an antibody, or an antigen-binding fragment thereof, of the invention is suitable for the use as a vector for stable transfection.

In certain embodiments the polynucleotide encoding an antibody, or an antigen-binding fragment thereof, of the invention is suitable for the use as a vector that enables production of the antibody, or antigen-binding fragment thereof, in a host cell.

In certain embodiments, the invention relates to a host cell comprising the polynucleotide of the invention.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

In certain embodiments the host cell is directly or indirectly used in therapy (e.g., cell therapy).

In certain embodiments a method for cell therapy comprises the steps of (i) obtaining a cell from a subject; (ii) transform the cell using a tool (e.g. a vector) comprising the polynucleotide of the invention and/or transform the cell to produce the antibody of the invention; and (iii) administering the transformed cell to a subject.

In certain embodiments, the invention relates to a method for producing an antibody comprising culturing the host cell of the invention.

This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide encoding a correspondingly chosen antibody can be subcloned into an appropriated vector, wherein the recombinant polypeptide is to be expressed in an organism being able for an expression, for example in bacteria. Thus, the expressed recombinant protein can be intra-peritoneally injected into a mouse and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein or a conserved region, in particular the regions of the antibodies provided herein, can be obtained by directly immunizing the animal by directly injecting intramuscularly the vector expressing the desired polypeptide or a protein, in particular the epitope of the antibodies of the invention. The amount of obtained specific antibody can be quantified using an ELISA, which is also described herein below. Further methods for the production of antibodies are well known in the art, see, e.g. Harlow and Lane, 1988, CSH Press, Cold Spring Harbor.

In a particular embodiment, the method of producing an antibody comprises culturing the host cell of the invention under conditions suitable to allow efficient production of the antibody of the invention.

In one such embodiment, a host cell comprises (e.g., has been transformed with): (1) a vector comprising a nucleic acid that encodes an amino acid sequence comprising the VL of the antibody and an amino acid sequence comprising the VH of the antibody of the invention, or (2) a first vector comprising a nucleic acid that encodes an amino acid sequence comprising the VL of the antibody and a second vector comprising a nucleic acid that encodes an amino acid sequence comprising the VH of the antibody of the invention. In one embodiment, the host cell is eukaryotic, e.g. a Chinese Hamster Ovary (CHO) cell or lymphoid cell (e.g., YO, NSO, Sp20). In one embodiment, a method of making an antibody specifically binding to a conserved region of the coronavirus S protein, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523; Charlton, 2003, Methods in Molecular Biology, Vol. 248; BKC Lo, 2003, Humana Press, pp. 245-254. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, 2004, Nat. Biotech. 22:1409-1414, and Li et al., 2006, Nat. Biotech. 24:210-215.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177; 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are macaque kidney CVI line transformed by SV40 (COS-7); human embryonic kidney line (293 or 293 cells as described, e.g., in Graham et al., 1997, J. Gen Viral. 36:59); baby hamster kidney cells (BHK); mouse sertoli cells (TM4 cells as described, e.g., in Mather, 1980, Biol. Reprod. 23:243-251); macaque kidney cells (CV I); African green macaque kidney cells (VER0-76); human cervical carcinoma cells (HELA); canine kidney cells (MDCK; buffalo rat liver cells (BRL 3A); human lung cells (WI38); human liver cells (Hep G2); mouse mammary tumor (MMT 060562); TRI cells, as described, e.g., in Mather et al., 1982, Annals N. Y Aead. Sei. 383:44-68; MRC 5 cells; and FS4 cells. Other useful mammalian host cell lines include Chinese hamster ovary (CHO) cells, including DHFR CHO cells (Urlaub et al., 1980, Proc. Natl. Acad. Sc. USA 77:4216); and myeloma cell lines such as YO, NSO and Sp2/0. For a review of certain mammalian host cell lines suitable for antibody production, see, e.g., Yazaki and Wu, Methods in Molecular Biology, Vol. 248 BKC Lo, 2003., Humana Press, pp. 255-268.

In certain embodiments, the invention relates to the antibody, or antigen-binding fragment thereof, of the invention, the polynucleotide of the invention or the host cell of the invention for use as a medicament.

For example, the antibody, or antigen-binding fragment thereof, of the invention, the polynucleotide of the invention or the host cell of the invention may be used as a medicament in treatment and/or prevention in the form of a pharmaceutical composition.

The term “pharmaceutical composition”, as used herein, refers to a preparation which is in such form as to permit the biological activity of an active ingredient contained therein to be effective, and which contains no additional components which are unacceptably toxic to a subject to which the formulation would be administered.

The term “treatment” (and grammatical variations thereof such as “treat” or “treating”), as used herein, refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some embodiments, antibodies of the invention are used to delay development of a disease or to slow the progression of a disease.

The term “prevention”, as used herein, relates to the capacity to prevent, minimize or hinder the onset or development of a disorder, disease or condition before its onset.

In some embodiments comprising the polynucleotide of the invention, the pharmaceutically acceptable carrier is or enables formation of a retrovirus, an adenovirus, an adeno associated virus, an envelope protein pseudotyping a viral vector, a replication-competent vector, cis and trans-acting elements, a herpes simplex virus and/or parts thereof.

In some embodiments comprising the host cell of the invention, the pharmaceutically acceptable carrier enables conservation and/or viability of cells.

In some embodiments, the pharmaceutical composition described herein (and any additional therapeutic agent) is administered systemically. In some embodiments, the pharmaceutical composition described herein (and any additional therapeutic agent) is administered locally. In some embodiments, the pharmaceutical composition described herein (and any additional therapeutic agent) is administered by any suitable means, including parenteral, intrapulmonary, and intranasal, and, if desired for local treatment, intralesional, intrauterine or intravesical administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some cases, an effective amount of the pharmaceutical composition described herein (and any additional therapeutic agent) can be any amount that reduces the severity, or occurrence, of symptoms of the disease, disorder and/or condition to be treated without producing significant toxicity to the subject. In some cases, an effective amount of the pharmaceutical composition described herein (and any additional therapeutic agent) can be any amount that reduces the number of diseased cells (e.g., dysregulated immune cells), pathogens and/or infected cells without producing significant toxicity to the subject.

The effective amount of the pharmaceutical composition described herein (and any additional therapeutic agent) can remain constant or can be adjusted as a sliding scale or variable dose depending on the subject's response to treatment. In some cases, the frequency of administration can be any frequency that reduces the severity, or occurrence, of symptoms of the disease, disorder and/or condition to be treated without producing significant toxicity to the subject. Various factors can influence the actual effective amount used for a particular application. For example, the frequency of administration, duration of treatment, use of multiple treatment agents, route of administration, and severity of the disease, disorder and/or condition may require an increase or decrease in the actual effective amount administered.

In some cases, the frequency of administration can be any frequency that reduces the number of diseased cells (e.g., dysregulated immune cells), pathogens and/or infected cells without producing significant toxicity to the subject. As with the effective amount, various factors can influence the actual frequency of administration used for a particular application. For example, the effective amount, duration of treatment, use of multiple treatment agents, route of administration, and severity of the disease, disorder and/or condition may require an increase or decrease in administration frequency.

In some cases, an effective duration for administering the pharmaceutical composition described herein (and any additional therapeutic agent) can be any duration that reduces the severity, or occurrence, of symptoms of the disease, disorder and/or condition to be treated without producing significant toxicity to the subject. In some cases, an effective duration for administering the pharmaceutical composition described herein (and any additional therapeutic agent) can be any duration that reduces the number of diseased cells (e.g., dysregulated immune cells), pathogens and/or infected cells without producing significant toxicity to the subject. Multiple factors can influence the actual effective duration used for a particular treatment. For example, an effective duration can vary with the frequency of administration, effective amount, use of multiple treatment agents, route of administration, and severity of the disease, disorder and/or condition being treated.

In certain embodiments, a course of treatment and/or the severity of the disease, disorder and/or condition being treated can be monitored. Any appropriate method can be used to determine whether or not the severity of a disease, disorder and/or condition is reduced. For example, the severity of a disease (e.g., inflammation) can be assessed in some embodiments using imaging techniques (with or without contrast), biopsy techniques, colonoscopy, sigmoidoscopy, digital rectal exam, blood assay, platelet assay, fecal assay, urine assay, endoscopic techniques, ELISA techniques, PCR-based techniques, blotting techniques (e.g., western blot), flow cytometry, genetic analysis (e.g., for gene rearrangements), and/or histological techniques at different time points. For example, the severity of an infection can be assessed using antibody techniques, viral antigen detection tests, culturing techniques, ELISA techniques, PCR-based techniques (e.g., viral load test), blotting techniques (e.g., western blot), and/or histological techniques at different time points. Any appropriate method can be used to monitor the response to therapies with the pharmaceutical composition described herein and/or the antibody, or antigen binding fragment thereof, of the invention. For example, techniques to detect levels ingredients of the pharmaceutical composition (e.g. the antibody, or antigen-binding fragment thereof, of the invention, the polynucleotide of the invention, the host cell of the invention and/or a further therapeutic agent) including ELISA techniques, PCR-based techniques, blotting techniques (e.g., western blot), hybridization techniques (e.g., ISH) and/or histological techniques (e.g., IHC).

The pharmaceutical composition described herein (and any additional therapeutic agent) would be formulated, dosed, and administered in a fashion consistent with good medical practice. Factors for consideration in this context include the particular disorder being treated, the particular subject being treated, the clinical condition of the individual patient, the cause of the disorder, the site of delivery of the agent, the method of administration, the scheduling of administration, and other factors known to medical practitioners. An antibody, or an antigen-binding fragment thereof, of the invention need not be, but is optionally formulated with one or more further therapeutic agents currently used to prevent or treat the disorder in question. The effective amount of such other agents depends on the amount of antibody, or antigen-binding fragment thereof, present in the composition, the type of disorder or treatment, and other factors for consideration discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody, or an antigen-binding fragment thereof, of the invention (when used alone or in combination with one or more other further therapeutic agents) will depend on the type of disease to be treated, the type of antibody, or antigen-binding fragment thereof, the severity and course of the disease, whether the antibody or antigen-binding fragment is administered for preventive or therapeutic purposes, previous therapy, the patient's clinical history and response to the antibody or antigen-binding fragment and the discretion of the attending physician.

The antibody, or antigen-binding fragment thereof, of the invention and/or the antibody used as a further therapeutic agent are/is suitably administered to the patient at one time or over a series of treatments. Depending on the type and severity of the disease, about 1 μg/kg to 15 mg/kg (e.g. 0.1 mg/kg-10 mg/kg) of antibody or antigen-binding fragment can be an initial candidate dosage for administration to the patient, whether, for example, by one or more separate administrations, or by continuous infusion. One typical daily dosage might range from about 1 μg/kg to 100 mg/kg or more, depending on the factors for consideration mentioned above. For repeated administrations over several days or longer, depending on the condition, the treatment would generally be sustained until a desired suppression of disease symptoms occurs. One exemplary dosage of the antibody or antigen-binding fragment would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose followed by one or more lower doses may be administered. However, other dosage regimens may be useful. The progress of this therapy is easily monitored by conventional techniques and assays.

In some embodiments pharmaceutical composition comprises the polynucleotide of the invention and the further therapeutic agent is a transfection enhancer, e.g., a transfection enhancer selected from the group of oligonucleotides, lipoplexes, polymersomes, polyplexes, dendrimers, inorganic nanoparticles and cell-penetrating peptides.

In some embodiments the pharmaceutical composition comprises the polynucleotide in the form of a vector genome in doses in the range from at least 106, 107, 108, 109, 1010, 1011, 1012, 1013, 1014, 1015, 1016, or more, vector genomes per kilogram (vg/kg) of the weight of the subject, to achieve a therapeutic effect.

In some embodiments the pharmaceutical composition comprises host cell and the further therapeutic agent is a cell signaling molecule such as a hormone, a neurotransmitter or a cytokine (see, e.g., Ding, Z. et al., 2017 Sci Rep 7, 12168)

In certain embodiments the pharmaceutical composition comprises a clinically relevant number or population of host cells and/or stem cell therapy cells, e.g., at least 104, 105,106, 107, 108, 109, typically more than 109 or at least 10¹⁰ cells per dose. The number of cells will depend upon the ultimate use for which the pharmaceutical composition is intended as will the type of cell. In some embodiments, the pharmaceutical composition will contain greater than 70%, generally greater than 80%, 85% and 90-95% of the host cells and/or stem cell therapy cells. For uses provided herein, the cells are typically in a volume of a liter or less, can be 500 ml or less, even 250 ml or 100 ml or less. Hence the density of the desired cells is typically be greater than 10⁶ cells/ml and generally is greater than 10⁷ cells/ml. The clinically relevant number of host cells can be apportioned into multiple infusions that cumulatively equal or exceed 10⁹, 10¹⁰ or 10¹¹ cells.

The total dose of the host cell of the invention for one therapy cycle is typically about 1×10⁴ cells/kg to 1×10¹⁰ cells/kg host cells or more, depending on the factors for consideration mentioned above.

In some embodiments any pharmaceutical composition is used for any of the methods (e.g., treatments) described herein.

The composition herein may also contain more than one active ingredient as necessary for the particular indication being treated, preferably those with complementary activities that do not adversely affect each other.

In certain embodiments, the invention relates to a pharmaceutical composition comprising at least two antibodies, or antigen-binding fragments thereof, wherein a first antibody, or antigen-binding fragment thereof is the antibody, or antigen-binding fragment thereof, of the invention, and wherein a second antibody or antigen fragment thereof, specifically binds to RBD.

Accordingly, the invention is at least in part based on the synergizing effect of simultaneously binding to conserved region and a second region of a coronavirus S protein.

In certain embodiments, the invention relates to the pharmaceutical composition of the invention, wherein the pharmaceutical composition further comprises a pharmaceutically acceptable carrier.

The term “pharmaceutically acceptable carrier”, as used herein, refers to an ingredient in a pharmaceutical formulation, other than an active ingredient, which is nontoxic to a subject. A pharmaceutically acceptable carrier includes, but is not limited to, a buffer, excipient, stabilizer, or preservative.

Pharmaceutical compositions of the antibody/antibodies, or antigen-binding fragment(s) thereof, the polynucleotide, the host cell as described herein are prepared by mixing such antibody/antigen-binding fragment/polynucleotide/host cell having the desired degree of purity with one or more optional pharmaceutically acceptable carriers (Osol et al., 1980 Remington's Pharmaceutical Sciences 16th edition), in certain examples, in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US 2005/0260186 and US 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter formulations including a histidine-acetate buffer.

In certain embodiments, the invention relates to the antibody, or antigen-binding fragment thereof, of the invention, the polynucleotide of the invention, the host cell of the invention or the pharmaceutical composition of the invention for use in the treatment and/or prevention of a coronavirus infection.

The term “coronavirus infection”, as used herein, refers to an infection with a virus from the family of Coronaviridae, preferably a coronavirus selected from group comprising MERS-COV, SARS-COV and SARS-COV-2 (or any variant thereof). A coronavirus infection can be assessed for example by an antigen test, pcr test, symptoms and/or a history of contact with a (likely) infected subject.

The coronavirus infection can cause a respiratory tract infection resulting in a disease or syndrome that is a respiratory syndrome. The respiratory syndrome can be a severe acute respiratory syndrome (SARS). In some embodiments, the SARS-COV-2 infection is at least one of the three clinical courses of infections can be distinguished: (1) mild illness with upper respiratory tract manifestations, (2) non-life-threatening pneumonia and, (3) severe condition with pneumonia, acute respiratory distress syndrome (ARDS), severe systemic inflammation, organ failures, cardiovascular complications.

As such the phrase “treatment and/or prevention of a coronavirus infection” can also mean “treatment and/or prevention of a symptom of a coronavirus infection”.

In some embodiments, the coronavirus infection described herein is caused by virus selected from the group consisting of alpha genera, beta genera, gamma genera and delta genera, preferably by the beta genera.

In some embodiments, the coronavirus infection described herein is caused by virus selected from the group consisting of Embecovirus, Hibecovirus, Merbecovirus, Nobecovirus, and Sarbecovirus.

The antibodies and antigen-binding fragment described herein are effective against a broad range of viruses. This enables effective therapy without the need of specific testing, in mutating or emerging viruses and/or in co-infections.

Accordingly, the invention is at least in part based on the broad applicability of the antibodies and antigen-binding fragments described herein.

In certain embodiments, the invention relates to a method for treatment and/or prevention of a coronavirus infection in a subject, the method comprising delivering a therapeutically effective amount of the antibody, or antigen-binding fragment thereof, of the invention, the polynucleotide of the invention, the host cell of the invention or the pharmaceutical composition of the invention to a subject.

In certain embodiments, the invention relates to the antibody, or antigen-binding fragment thereof, for use of the invention, the polynucleotide for use of the invention, the host cell for use of the invention or the pharmaceutical composition for use of the invention or the method for treatment and/or prevention of the invention, wherein the coronavirus is SARS-COV-2.

The term “SARS-COV-2”, as used herein, includes any SARS-COV-2 variant, such as known SARS-COV-2 variants and future SARS-COV-2 variants, preferably the SARS-CoV-2 variants described herein.

The antibodies and antigen-binding fragment described herein are effective against a broad range of SARS-COV-2 variants. This enables effective therapy in without the need of specific testing, in mutating or emerging viruses and/or in co-infections.

Accordingly, the invention is at least in part based on the broad applicability of the antibodies and antigen-binding fragments described herein.

In certain embodiments, the invention relates to a method for identifying a coldspot antibody or an antigen-binding fragment thereof, the method comprising the steps of:
a) identifying a conserved region in an antigen;
b) generating a peptide comprising or consisting of the conserved region of the antigen or a fragment of the conserved region;
c) bringing at least two antibodies or antigen-binding fragments thereof of a convalescent subject into contact with the conserved region, wherein the convalescent subject is a subject that was previously exposed to the antigen; and
d) identifying at least one coldspot antibody or antigen-binding fragment thereof from the antibodies of the convalescent subject, wherein the coldspot antibody or antigen-binding fragment thereof has at least one better coldspot binding property than at least one second antibody or antigen-binding fragment thereof of the antibodies or antigen-binding fragments thereof of the convalescent subject, wherein a better coldspot binding property is a property selected from the group consisting of: i) higher binding affinity to the peptide; ii) cross-specificity to the peptide and to a second antigen, wherein the second antigen is a related antigen; and iii) specific binding to the peptide and inhibition of a biologic activity of the antigen.

The term “conserved region”, as used herein, in the context of a method for identifying a coldspot antibody or an antigen-binding fragment thereof refers to a region that can be bound by an antibody and is evolutionarily conserved compared to an antigen from a phylogenetically related source.

The term “antigen”, as used herein, refers to a molecule that provokes an immune response, preferably an immune response involving antibody production. As such, any macromolecule, in particular proteins or peptides, can serve as an antigen. In a preferred embodiment, the antigen is a peptide. The antigen may for example be part of a cancer (e.g., a tumor) or a part of a pathogen (e.g., a virus). An antigen from a “related source” therefore depends on the type of the antigen source. For example, if the antigen is part of a cancer, the “related source” may be a cancer of the same type, a primary tumor of the cancer and/or a metastasis of the cancer. If the antigen is part of a pathogen, the “related source” may be a pathogen from the same taxonomic clade, species, subgenus, genus and/or family.

The peptide generated corresponding to the conserved region can be a short peptide consisting of the conserved region or a protein (subunit) comprising one or more conserved regions, wherein the conserved region is accessible for antibody binding. The peptide generated corresponding to the conserved region can also only comprise a fragment of the conserved region, wherein the fragment of the conserved region is large enough to ensure specific binding. In some embodiments, the fragment of the conserved region comprises or consists of at least 5, at least 6, at least 7, at least 8, at least 9, at least 10, at least 11, at least 12, at least 13, at least 14, at least 15, at least 16, or at least 17 amino acids of the conserved region. In some embodiments, the fragment of the conserved region comprises or consists of at least 50%, at least 60%, at least 70%, at least 80%, at least 90%, or at least 95% of the amino acid length of the conserved region.

The term “convalescent subject”, as used herein, refers to a subject that was exposed to the antigen. In some embodiments, the convalescent subject is healthier than during at least one phase of exposure to the antigen, for example the convalescent subject is recovered or partially recovered from a disease or disorder induced by the antigen comprising pathogen or recovered from a reaction to a vaccine.

The term “related antigen”, as used herein, refers to an antigen from phylogenetically related source.

The term “biologic activity of the antigen”, as used herein, refers to any biologic activity of the antigen itself or any biologic activity of the source of the antigen (e.g. a pathogen) that can be inhibited by binding to the antigen. For example, the biologic activity of the antigen can be to enable entry of the virus into target cells (viral fusion).

The inventors found that the method described herein is particularly effective for identifying antibodies that bind to coldspot regions.

Accordingly, the invention is at least in part based on the finding that antibodies identified by the method of the invention, is particularly effective in providing cross-specific and/or long-term effective antibodies.

In certain embodiments, the invention relates to the method of the invention, wherein identifying conserved regions of an antigen comprises comparison of a sequence of the antigen to a sequence of a related antigen.

In certain embodiments, the invention relates to the method of the invention, wherein the conserved region comprises at least 17 consecutive aa with frequency of substitutions <0.1%.

The inventors found that a threshold of >17 consecutive aa in length with less than 0.1% of amino acid changes in the available database of over 10 mio virus sequences is effective to identify conserved regions as described herein.

In certain embodiments, the invention relates to the method of the invention, wherein step c) comprises bringing at least two antibodies or antigen-binding fragments thereof of a convalescent subject into contact with the conserved region in an ELISA.

In certain embodiments, the invention relates to the method of the invention, wherein step d) comprises FACS (fluorescence-activated cell sorting).

In certain embodiments, the invention relates to the method of the invention, wherein the antibodies or antigen-binding fragments thereof of a convalescent subject are memory B cell-derived antibodies.

In certain embodiments, the invention relates to the method of the invention, wherein identifying a conserved region further comprises identifying a secondary, tertiary and/or quaternary structure of the conserved region in the antigen.

In certain embodiments, the invention relates to the method of the invention, wherein the antigen is a pathogen and wherein the convalescent subject is a subject that was previously exposed to the pathogen, a phylogenetically related pathogen, a vaccine to the pathogen and/or or an attenuated and/or inactivated version of the pathogen or to a phylogenetically related pathogen.

In certain embodiments, the invention relates to the method of the invention, wherein the antigen is a cancer antigen.

The inventors further developed an innovative flow cytometry approach combining positive selection of B cells with antibodies binding to SD1-RBD fusion protein with negative selection of B cells with antibodies binding solely to RBD.

As such, a combined protein subdomain was used to identify antibodies and a single (non-coldspot) subdomain was used to exclude antibodies that do not bind to the coldspot part of the combined protein subdomain.

In certain embodiments, the invention relates to an antibody, or antigen-binding fragment thereof, specifically binding to the RBD region of a coronavirus S protein, the antibody or antigen-binding fragment thereof comprising:
a) a variable heavy (VH) chain comprising CDR1 as defined in SEQ ID NO: 173, CDR2 as defined in SEQ ID NO: 174 and CDR3 as defined in SEQ ID NO: 175 and a variable light (VL) chain comprising CDR1 as defined in SEQ ID NO: 176, CDR2 comprising or consisting of the sequence of DAS and CDR3 as defined in SEQ ID NO: 177;
b) a variable heavy (VH) chain comprising CDR1 as defined in SEQ ID NO: 178, CDR2 as defined in SEQ ID NO: 179 and CDR3 as defined in SEQ ID NO: 180 and a variable light (VL) chain comprising CDR1 as defined in SEQ ID NO: 181, CDR2 comprising or consisting of the sequence of GAS and CDR3 as defined in SEQ ID NO: 182;
c) a variable heavy (VH) chain comprising CDR1 as defined in SEQ ID NO: 183, CDR2 as defined in SEQ ID NO: 184 and CDR3 as defined in SEQ ID NO: 185 and a variable light (VL) chain comprising CDR1 as defined in SEQ ID NO: 186, CDR2 comprising or consisting of the sequence of EVS and CDR3 as defined in SEQ ID NO: 187;
d) a variable heavy (VH) chain comprising CDR1 as defined in SEQ ID NO: 188, CDR2 as defined in SEQ ID NO: 189 and CDR3 as defined in SEQ ID NO: 190 and a variable light (VL) chain comprising CDR1 as defined in SEQ ID NO: 191, CDR2 comprising or consisting of the sequence of GNN and CDR3 as defined in SEQ ID NO: 192;
e) a variable heavy (VH) chain comprising CDR1 as defined in SEQ ID NO: 193, CDR2 as defined in SEQ ID NO: 194 and CDR3 as defined in SEQ ID NO: 195 and a variable light (VL) chain comprising CDR1 as defined in SEQ ID NO: 196, CDR2 comprising or consisting of the sequence of LGS and CDR3 as defined in SEQ ID NO: 197;
f) a variable heavy (VH) chain comprising CDR1 as defined in SEQ ID NO: 198, CDR2 as defined in SEQ ID NO: 199 and CDR3 as defined in SEQ ID NO: 200 and a variable light (VL) chain comprising CDR1 as defined in SEQ ID NO: 201, CDR2 comprising or consisting of the sequence of EDN and CDR3 as defined in SEQ ID NO: 202;
g) a variable heavy (VH) chain comprising CDR1 as defined in SEQ ID NO: 203, CDR2 as defined in SEQ ID NO: 204 and CDR3 as defined in SEQ ID NO: 205 and a variable light (VL) chain comprising CDR1 as defined in SEQ ID NO: 206, CDR2 comprising or consisting of the sequence of GAS and CDR3 as defined in SEQ ID NO: 207;
h) a variable heavy (VH) chain comprising CDR1 as defined in SEQ ID NO: 208, CDR2 as defined in SEQ ID NO: 209 and CDR3 as defined in SEQ ID NO: 210 and a variable light (VL) chain comprising CDR1 as defined in SEQ ID NO: 211, CDR2 comprising or consisting of the sequence of AAS and CDR3 as defined in SEQ ID NO: 212; or
i) a variable heavy (VH) chain comprising CDR1 as defined in SEQ ID NO: 213, CDR2 as defined in SEQ ID NO: 214 and CDR3 as defined in SEQ ID NO: 215 and a variable light (VL) chain comprising CDR1 as defined in SEQ ID NO: 216, CDR2 comprising or consisting of the sequence of GAS and CDR3 as defined in SEQ ID NO: 217.

In certain embodiments, the invention relates to the antibody, or antigen-binding fragment thereof, of the invention, the antibody, or antigen-binding fragment thereof, comprising:
a) a variable heavy (VH) chain sequence comprising the amino acid sequence of SEQ ID NO: 280 or a sequence having at least 85%, 86%, 87%, 88%, 89%, 90%, 91%, 92%, 93%, 94%, 95%, 96%, 97%, 98% or 99% sequence identity to SEQ ID NO: 280; and a variable light (VL) chain sequence comprising the amino acid sequence of SEQ ID NO: 281 or a sequence having at least 85%, 86%, 87%, 88%, 89%, 90%, 91%, 92%, 93%, 94%, 95%, 96%, 97%, 98% or 99% sequence identity to SEQ ID NO: 281;
b) a variable heavy (VH) chain sequence comprising the amino acid sequence of SEQ ID NO: 282 or a sequence having at least 85%, 86%, 87%, 88%, 89%, 90%, 91%, 92%, 93%, 94%, 95%, 96%, 97%, 98% or 99% sequence identity to SEQ ID NO: 282; and a variable light (VL) chain sequence comprising the amino acid sequence of SEQ ID NO: 283 or a sequence having at least 85%, 86%, 87%, 88%, 89%, 90%, 91%, 92%, 93%, 94%, 95%, 96%, 97%, 98% or 99% sequence identity to SEQ ID NO: 283;
c) a variable heavy (VH) chain sequence comprising the amino acid sequence of SEQ ID NO: 284 or a sequence having at least 85%, 86%, 87%, 88%, 89%, 90%, 91%, 92%, 93%, 94%, 95%, 96%, 97%, 98% or 99% sequence identity to SEQ ID NO: 284; and a variable light (VL) chain sequence comprising the amino acid sequence of SEQ ID NO: 285 or a sequence having at least 85%, 86%, 87%, 88%, 89%, 90%, 91%, 92%, 93%, 94%, 95%, 96%, 97%, 98% or 99% sequence identity to SEQ ID NO: 285;
d) a variable heavy (VH) chain sequence comprising the amino acid sequence of SEQ ID NO: 286 or a sequence having at least 85%, 86%, 87%, 88%, 89%, 90%, 91%, 92%, 93%, 94%, 95%, 96%, 97%, 98% or 99% sequence identity to SEQ ID NO: 286; and a variable light (VL) chain sequence comprising the amino acid sequence of SEQ ID NO: 287 or a sequence having at least 85%, 86%, 87%, 88%, 89%, 90%, 91%, 92%, 93%, 94%, 95%, 96%, 97%, 98% or 99% sequence identity to SEQ ID NO: 287;
e) a variable heavy (VH) chain sequence comprising the amino acid sequence of SEQ ID NO: 288 or a sequence having at least 85%, 86%, 87%, 88%, 89%, 90%, 91%, 92%, 93%, 94%, 95%, 96%, 97%, 98% or 99% sequence identity to SEQ ID NO: 288; and a variable light (VL) chain sequence comprising the amino acid sequence of SEQ ID NO: 289 or a sequence having at least 85%, 86%, 87%, 88%, 89%, 90%, 91%, 92%, 93%, 94%, 95%, 96%, 97%, 98% or 99% sequence identity to SEQ ID NO: 289;
f) a variable heavy (VH) chain sequence comprising the amino acid sequence of SEQ ID NO: 290 or a sequence having at least 85%, 86%, 87%, 88%, 89%, 90%, 91%, 92%, 93%, 94%, 95%, 96%, 97%, 98% or 99% sequence identity to SEQ ID NO: 290; and a variable light (VL) chain sequence comprising the amino acid sequence of SEQ ID NO: 291 or a sequence having at least 85%, 86%, 87%, 88%, 89%, 90%, 91%, 92%, 93%, 94%, 95%, 96%, 97%, 98% or 99% sequence identity to SEQ ID NO: 291;
g) a variable heavy (VH) chain sequence comprising the amino acid sequence of SEQ ID NO: 292 or a sequence having at least 85%, 86%, 87%, 88%, 89%, 90%, 91%, 92%, 93%, 94%, 95%, 96%, 97%, 98% or 99% sequence identity to SEQ ID NO: 292; and a variable light (VL) chain sequence comprising the amino acid sequence of SEQ ID NO: 293 or a sequence having at least 85%, 86%, 87%, 88%, 89%, 90%, 91%, 92%, 93%, 94%, 95%, 96%, 97%, 98% or 99% sequence identity to SEQ ID NO: 293;
h) a variable heavy (VH) chain sequence comprising the amino acid sequence of SEQ ID NO: 294 or a sequence having at least 85%, 86%, 87%, 88%, 89%, 90%, 91%, 92%, 93%, 94%, 95%, 96%, 97%, 98% or 99% sequence identity to SEQ ID NO: 294; and a variable light (VL) chain sequence comprising the amino acid sequence of SEQ ID NO: 295 or a sequence having at least 85%, 86%, 87%, 88%, 89%, 90%, 91%, 92%, 93%, 94%, 95%, 96%, 97%, 98% or 99% sequence identity to SEQ ID NO: 295; or
i) a variable heavy (VH) chain sequence comprising the amino acid sequence of SEQ ID NO: 296 or a sequence having at least 85%, 86%, 87%, 88%, 89%, 90%, 91%, 92%, 93%, 94%, 95%, 96%, 97%, 98% or 99% sequence identity to SEQ ID NO: 296; and a variable light (VL) chain sequence comprising the amino acid sequence of SEQ ID NO: 297 or a sequence having at least 85%, 86%, 87%, 88%, 89%, 90%, 91%, 92%, 93%, 94%, 95%, 96%, 97%, 98% or 99% sequence identity to SEQ ID NO: 297.

The inventors found that (despite the RBD subdomain not being a conserved region) the antibodies, or antigen-binding fragments thereof, specifically binding to the RBD subdomain of the coronavirus S protein have a broad effective range and/or show synergistic effect with antibodies or antigen-binding fragments thereof binding to a conserved region such as a conserved region as defined by SEQ ID NO: 16 and/or SEQ ID NO: 298.

“a,” “an,” and “the” are used herein to refer to one or to more than one (i.e., to at least one, or to one or more) of the grammatical object of the article.

“or” should be understood to mean either one, both, or any combination thereof of the alternatives.

“and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. “preferably” means one option out of a series of options not excluding other options. “e.g.” means one example without restriction to the mentioned example. By “consisting of” is meant including, and limited to, whatever follows the phrase “consisting of.”

The terms “about” or “approximately”, as used herein, refer to “within 20%”, more preferably “within 10%”, and even more preferably “within 5%”, of a given value or range.

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While embodiments of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1. Discovery of virus-neutralizing coldspot antibodies.

(A) On top, cartoon diagram of the SARS-COV-2 spike with highlighted the coldspot areas at the fusion peptide (FP,), heptad repeat 2 region (HR2,), and subdomain 1 (SD1). The thick horizontal lines indicate the location of all coldspots (see also FIG. 6). At the bottom, amino acid changes in SARS-COV-2 variants. Each circle represents a single aa substitution over ancestral virus. (B) Structure of the SARS-COV-2 spike; FP (aa 814-838) and HR2 (aa 1142-1161) coldspots are in darker, in boxes, (PDB: 6XM4). (C) Graph shows ELISA measurements of convalescent plasma IgG reactivity to FP (top) or HR2 (bottom) peptides. Optical density units at 450 nm (OD, Y axis) and reciprocal plasma dilutions (X axis). Non-infected controls in black dotted; samples selected for cell sorting by flow cytometry are in in black. Two independent experiments. (D) Representative flow cytometry plots of B cells binding to fluorescently labeled FP (top) or HR2 (bottom) peptides. Numbers indicate percentage of double-positive cells in the gate. (E) Number of heavy and light chain V gene somatic mutations of antibodies to the FP (top) or HR2 (bottom) peptides. (F) Heatmaps with ELISAs EC50 values of monoclonal antibodies binding to the S of CoVs (top) and to the FP and HR2 peptides (bottom) corresponding to the CoV species, whose genus is indicated by Greek letters. The monoclonal antibodies to the HR2 region S2P6 (D. Pinto et al., Science. 373, 1109-1116 (2021).) and CV3-25 (M. F. Jennewein, et al., Cell Rep. 36, 109353 (2021).) were assayed alongside for comparison. Cross indicates not tested. Two experiments. (G) Graph with IC50 values of monoclonal antibodies neutralizing pseudoviruses corresponding to the indicated VOC. Two experiments. (H) ACE2 binding to ancestral S in ELISA in the presence of select FP and HR2 antibodies. Dotted line represents the limit of detection. Two experiments. (I) Inhibition of cell fusion by FP and HR2 antibodies. Two experiments. (J and K) fp.006 and hr2.016 antibodies protect in vivo. Top, diagram of the experiment's timeline. Middle, mouse weight over time after challenge with ancestral SARS-COV-2 of mice treated with antibodies either 24 hours before ((J); n=6 per group, p=0.0022 for both fp.006 and hr2.016 versus isotype at day 7), or 2 hours after ((K); n=5 per group, p=0.0079 for both fp.006 and hr2.016 versus isotype at day 7) the infection. Mann-Whitney U test, standard deviation is shown. At the bottom, representative lung images at day 7.

FIG. 2. Crystal structure of fp.006 bound to the SARS-COV-2 FP.

(A) Overview of the complex structure of fp.006 Fab (surface representation; heavy chain in gray, light chain in light grey) bound to the SARS-COV-2 FP (cartoon) with interacting side chains represented as sticks. (B) Visualization of FP residues F823, E819, and R815 resting in a deep groove formed at the antibody paratope, with coloring as in (A). (C) Overlay of the fp.006-FP crystal structure with a cryo-EM structure of the SARS-COV-2 prefusion S trimer (PDB: 6VXX). Models were aligned on Ca atoms of FP residues 818-822 (helical in both structures) with a root mean square deviation of 0.97 Å. (D) Residue-level interactions between FP residue R815 and the antibody heavy chain include hydrogen bond formation with N31 and a cation-IT interaction with Y52A. (E) Water-mediated interactions between FP residue E819 and heavy chain residues Y52A, N56, and F97. Water molecules are shown as spheres. (F) van der Waals contacts between FP residue F823 (stick) and residues that comprise a groove at the heavy and light chain interface (surfaces). (G) Interactions between FP residue D820 and fp.006 CDRH2 residues include a salt bridge with R55 and additional hydrogen bond formation with N56. Hydrogen bonds, salt bridges, and cation-IT interactions are shown as dashed lines. (H) Flow cytometry detection of anti-FP and anti-HR2 antibody binding to SARS-COV-2 S expressed on 293T cells. Left, representative FACS plots (pre-gated on live-singlets-GFP+ cells). Black lines indicate isotype control in the presence (continuous line) or absence (dotted line) of soluble ACE2. Right, quantification of the geometric mean fluorescent intensity (gMFI; n=4). Two-tailed paired t-test: *p<0.05, **p<0.01, ***p<0.001 and ****p<0.0001; standard deviation is shown.

FIG. 3. Discovery of broadly cross-specific antibodies to SD1 and RBD.

(A) Structure of the SARS-COV-2 S. S protomer with RBD up (left) or down (middle) and S trimer with two down and one up (right; PDB: 6XM4). SD1 and RBD are encircled. (B) Graph shows ELISAs measuring plasma IgG reactivity to SD1-RBD. Negative controls in black; samples selected for sorting in dark gray. Mean of two independent experiments. 82.1% of the plasma samples were positive (4SD higher than the average AUC of the controls) (C) Representative flow cytometry plot of B cells binding to fluorescently labeled SD1-RBD. Percentage refers to gated cells. (D and E) ELISAs measuring the reactivity of monoclonal antibodies to SD1-RBD (D) and to RBD (E). Mean of two independent experiments. (F) Heatmaps with the binding (EC50) of SD1 monoclonal antibodies to S (top) or SD1-RBD (bottom) proteins corresponding to SARS-COV-2 VOC. Two experiments. (G) Graph shows normalized relative luminescence values in cell lysates of 293TACE2 cells after infection with ancestral SARS-COV-2 pseudovirus in the presence of increasing concentrations of broadly crossreactive SD1 monoclonal antibodies. At least two independent experiments.

FIG. 4. Cryo-EM structure of sd1.040 in complex with SARS-COV-2 S.

(A) ACE2 binding to ancestral S in ELISA in the presence of sd1.040 or C121 control antibody. Representative of two experiments. (B) Structure of the sd1.040-S complex. Spike SD1 and RBD regions are shown as surface representation and colored light and dark gray, respectively. The sd1.040 Fab heavy chain (dark gray) and light chain (light gray) are shown as cartoon. The S N331-glycan that interacts with the sd1.040 Fab is shown as gray spheres. Inset: sd1.040 binding orientation on trimeric S shows clashes. (C and D) Surface rendering of sd1.040 epitope is highlighted on the SD1 and RBD surfaces, with sd1.040 CDR loops shown (ribbon). The majority of sd1.040 contacts are mediated by CDRH2, CDRL1 and CDRL3 loops. (E) Surface plasmon resonance (SPR) experiment showing the binding of sd1.040 Fab to ancestral SD1-RBD or S. (F) Antibody sd1.040 prevents ACE2-induced rearrangements. Flow cytometry detection of fp.006 binding to ancestral SARS-COV-2 S expressed on 293T cells. Left, representative FACS plots. Black lines indicate isotype control in the presence (continuous line) or absence (dotted line) of soluble ACE2. Right, quantification of the geometric mean fluorescent intensity (gMFI; n=4). Two-tailed paired t-test: *p<0.05, **p<0.01, ***p<0.001 and ****p<0.0001.

FIG. 5. In vitro neutralization and mouse protection by the bispecific antibody CoV-X4042

(A) SPR assay of the sequential binding of immobilized antibodies to SD1-RBD protein followed by either sd1.040, rbd.042 or CoV-X4042. (B) SPR analysis showing that both arms of CoV-X4042 bind simultaneously to the same SD1-RBD molecule, since avidity is retained at decreasing SD1-RBD concentrations. Increasing normalized kd values indicate loss of avidity. Solid lines, IgG; dotted lines, Fab (see also FIG. 12A). (C) Normalized relative luminescence values in cell lysates of 293TACE2 cells after infection with ancestral SARS-COV-2 pseudovirus in the presence of increasing concentrations of CoV-X4042 or its parental monoclonal antibodies individually or as a cocktail. Isotype control represented by empty circles. (D) Graph with IC50 values of bispecific and parental monoclonal antibodies neutralizing pseudoviruses corresponding to the indicated VOC. Mean of two independent experiments. (E) In vitro neutralization of SARS-COV-2 by CoV-X4042. (F) CoV-X4042 protects in vivo. Top, diagram of the experiment's timeline. Bottom, mouse weight over time after challenge, with ancestral SARS-COV-2, of mice treated with antibodies either 24 hours before (PRE; n=5 per group, p=0.0079), or 2 hours after (POST; n=5 per group, p=0.0079; day 7) the infection. Mann-Whitney U test, standard deviation is shown. (G) CoV-X4042 reduces viral titers in the lungs. Mice were treated with antibodies 24 hours before infection and virus titers evaluated on day 3 (n=5 per group p=0.0079 with both ancestral and Omicron BA.1; Mann-Whitney U test, standard deviation is shown).

FIG. 6. Discovery of coldspot antibodies.

(A) Cartoon diagram of the SARS-COV-2 S with highlighted the coldspot regions at the fusion peptide (FP) and near heptad repeat 2 (HR2), as well as subdomain 1 (SD1). The thick horizontal lines underneath indicate the location of the coldspots (defined as >17 consecutive aa with frequency of substitutions <0.1%), and the histograms show the frequency of aa changes throughout S at different time points: 31.12.2020 (671,006), 31.12.2021 (7,526,116) and 29.04.2022 (10,480,461 sequences from GISAID). (B) ELISA reactivity of plasma IgG antibodies to FP (top) and HR2 (middle) peptides or to SARS-COV-2 RBD (bottom). SARS-COV-2 (n=67) and common cold coronavirus (n=6) convalescent samples are compared to vaccinees (mRNA: BNT162b2 n=11, purple; mRNA-1273 n=5, pink; inactivated virus: Sinovac n=2; Sinopharm n=24adenovirus: ChAdOx1-S n=19; Ad26.COV2.S n=4), and to non-infected controls (n=25). Area Under the Curve (AUC), average of two experiments. (C) Gating strategy for sorting peptide-specific or SD1-enriched B cells by flow cytometry. (D) Identification of FP-peptide specific (top) and HR2-peptide specific (bottom) B cells by flow cytometry. Numbers indicate percentage of gated double-positive cells. (E) Clonal analysis of antibody sequences derived from peptide-specific B cells identified in d. Pie charts show the total number of antibody sequences (center); the size of the slices corresponds to the number of clonally related sequences. White slices indicate antibody sequences that are not part of a clone.

FIG. 7. Similarity of peptides at the FP and HR2.

(A) Phylogenetic analysis of coronavirus species based on the amino acid sequence of S. (B) Alignment of SARS-COV-2 FP and HR2 peptide sequences with those corresponding to related coronaviruses. In dark gray identical residues; in bright gray residues that are similar by side chain functionality.

FIG. 8. Binding cross-reactivity of FP and HR2 monoclonal antibodies.

(A and B) Graphs show ELISAs measuring monoclonal antibodies reactivity to S (A) and to coldspot peptides (B) of coronaviruses. Mean of two independent experiments.

FIG. 9. Structure of fp.006 in complex with peptide antigen and effects of ACE2 engagement

(A) Contributions of fp.006 heavy and light chain paratope residues to the binding of the fusion peptide. Residue number, identity, and buried surface area (BSA) are shown, and colored as in panel (C). (B) Per residue contributions of the FP to the fp.006 epitope. Conservation calculated from the 49 CoV sequences listed in FIG. 7 and BSA calculations per FP residue are listed. * Indicates that the BSA calculation does not include contacts mediated by water molecules. (C) Surface representation of the fp.006 paratope with interacting residues in grayscale according to their CDR loop identity. Non-interacting heavy chain and light chain residues are colored teal and light teal, respectively. (D) Surface representation of the fp.006 paratope with per residue lipophilicity calculations shown. (E) Representative electron density for a portion of the FP contoured at 1.70. (F) Overlay of complex structures between the SARS-COV-2 fusion peptide and fp.006 (this study; PDB 8D47), VN01H1 (PDB 7SKZ), and COV91-27 (PDB 8D6Z) illustrates a convergent binding mode independent of V gene usage. Structures are aligned on their fusion peptide chains. (G) Overlay of complex structures between the SARS-COV-2 fusion peptide and fp.006 (this study; PDB 8D47), COV44-62 (PDB 8D36), and 76E1 (PDB 7X9E) Fabs, illustrating that anti-FP mAbs of different poses bind to the same face of the amphipathic fusion peptide α-helix, which includes highly-conserved residues, which are shown as sticks. (H) Gating strategy for measuring the binding of monoclonal antibodies to the SARS-COV-2 S expressed by 293T cells by flow cytometry. (I) Normalized relative luminescence values in cell lysates of 293TACE2 cells after infection with ancestral SARS-COV-2 pseudovirus in the presence of increasing concentrations of FP or HR2 antibodies alone or as a cocktail with soluble ACE2. Isotype control in black. For the combination of fp.006+ACE2, P=0.0017 over fp.006 and P=0.0047 over ACE2; for the combination of fp.007+ACE2, P=0.0072 over fp.007 and P=0.2 over ACE2 (n=4; Welch's t-test, two-tailed).

FIG. 10. Discovery of SD1 and RBD antibodies.

(A) Graph shows ELISAs measuring plasma IgG reactivity to RBD. Negative controls in black. Mean of two independent experiments. (B) Correlation of plasma IgG reactivities to SD1-RBD and RBD. Pearson correlation. (C) Identification of SD1-enriched B cells by flow cytometry. (D and E) Graphs show ELISAs measuring monoclonal antibodies reactivity to SD1-RBD (D) and S (E) corresponding to SARS-CoV-2 VOC. Mean of two independent experiments. (F) Heatmaps with the binding (EC50) of RBD monoclonal antibodies to S (top) or SD1-RBD (bottom) proteins corresponding to SARS-COV-2 VOC. Two experiments. (G) Graphs and summary heatmap for SD1 antibodies binding to SD1-RBD mutant proteins T572I and E583D. (H) Neutralization of ancestral SARS-COV-2 pseudovirus by antibodies to the RBD that are broadly crossreactive to VOC.

FIG. 11. Structure of sd1.040 in complex with S.

(A) Representative micrograph and 2D class averages selected from the sd1.040-S dataset. (B) 3D classification workflow and final refinement strategy. (C) Gold-standard FSC plot for the final cryo-EM density map. (D) Local resolution estimates calculated in cryoSPARC v3.1. (E) Close-up view of the sd1.040 Fab-S complex superimposed onto a prefusion S trimer (PDB 6VXX). Potential clashes with the NTD on the neighboring S1 protomer are denoted by a star. (F) Close-up views of the P008_60 Fab (PDB 7ZBU) and S3H3 Fab (PDB 7WD9) superposed onto a prefusion trimeric Spike. Antibody P008_60 shows prominent clashes with the adjacent NTD (star), while the binding orientation of S3H3 does not clash with neighboring protomers. (G and H) According to docking simulations, sd1.040 binds also to the NTD of the adjacent protomer. In (G), NTD moves from the position observed in the experimental structure of the free S to make room for antibody binding (sd1.040 in light gray bound to NTD in dark gray). In (H), comparison between NTD bound to sd1.040 in simulations (dark gray) and NTD in the context of free spike trimer (light gray).

FIG. 12. Binding and neutralizing properties of CoV-X4042.

(A) Both arms of CoV-X4042 bind simultaneously to the same SD1-RBD molecule. Diagram showing that at high SD1-RBD concentrations, monoclonal antibodies have avidity effects owing to intermolecular binding (which results in a slower kd), but this is not the case at low SD1-RBD concentrations, because bivalent binding to a single SD1-RBD is impossible. By contrast, the bispecific antibody has avidity at both high and low concentrations, since bivalent binding to its two epitopes on a single SD1-RBD is possible. ka is not affected by avidity. (B) Neutralization of VOC by CoV-X4042 and parental monoclonal antibodies.

FIG. 13 Broad neutralization by FP and HR2 monoclonal antibodies.

(A and B) Neutralization of ancestral SARS-COV-2 pseudovirus. Graphs show normalized relative luminescence values in cell lysates of 293TACE2 (A) or 293TACE2/TMPRSS2 (B) cells 48 hours after infection with nanoluc-expressing SARS-COV-2 pseudovirus in the presence of increasing concentrations of monoclonal antibodies to the FP (top) and HR2 (bottom). Isotype control and antibodies C135 (D. F. Robbiani, et al, Nature. 584, 437-442 (2020)), CV3-25 and S2P6 were assayed alongside for comparison; additional antibodies in grey. Mean of at least two independent experiments. (C) Same as in (A), but for SARS-COV-2 pseudoviruses corresponding to VOC. (D) Neutralization of SARS-COV-2 ancestral and Omicron BA.1 by Focus Reduction Neutralization Test (FRNT).

### EXAMPLES

### Methods

### Study Participants.

COVID-19 convalescent cohort: 67 individuals, who were diagnosed with COVID-19 at the Clinica Luganese Moncucco (CLM, Switzerland) between March and November of 2020, were enrolled in the study and written informed consent was obtained. Inclusion criteria were a SARS-COV-2 positive nasopharyngeal swab test by real-time reverse transcription-polymerase chain reaction (RT-PCR) or being a symptomatic close contact (same household) of a hospitalized participant, and age ≥18 years. Samples were 83-269 days after onset of symptoms. The study was performed in compliance with all relevant ethical regulations under study protocols approved by the Ethical Committee of the Canton Ticino (ECCT): CE-3428 and CE-3960.

Control cohort: 17 individuals (≥18 years) with absence of prior SARS-COV-2 infection or vaccination, as confirmed by negative serologic test, were enrolled between November 2020 and June 2021 and written informed consent was obtained (ECCT: CE-3428).

Vaccination cohort: individuals (≥18 years) with absence of prior SARS-COV-2 infection and who received either mRNA-based (n=11 for BNT162b2, samples obtained 75-136 days after second dose; n=5 for mRNA-1273, 85-120 days after second dose), adenovirus-based (n=19 for ChAdOx1-S, 90 days after second dose; n=4 for Ad26.COV2.S, 21 days after single dose), or inactivated virus-based (n=2 for Sinovac, 26-60 days after second dose; n=24 for Sinopharm, 6-60 days after second dose) COVID-19 vaccines were enrolled under approved protocols (ECCT: CE-3428 and CE-3960; Ethic Committee Karolinska Institutet: Dnr 2020-02646; Ethic Committee Tehran University of Medical Sciences: IR.TUMS.CHMC.REC.1399.098-B2; Ethical Committee for Clinical Experimentation of Regione Toscana Area Vasta Sud Est [CEASVE]: ID 18869). Controls to the adenovirus vaccinated group are pre-vaccination samples from the same participants.

Pre-pandemic common cold coronavirus convalescents: 6 samples from individuals with confirmed common cold CoV infection were obtained 6-375 days after symptoms onset at Policlinico San Matteo, Pavia (Institutional Review Board protocol number P_20200029440).

Blood sample processing and storage. Peripheral blood mononuclear cells (PBMCs) from COVID-19 convalescents were obtained by Histopaque density centrifugation and stored in liquid nitrogen in the presence of FBS and DMSO. Anticoagulated plasma was aliquoted and stored at −20 C or less. Prior to experiments, aliquots of plasma were heat-inactivated (56 C for 1 hour) and then stored at 4 C. Similarly, frozen plasma aliquots from non-infected, common cold-infected, and vaccinated individuals were stored at 4 C after heat-inactivation.

### Peptides and Recombinant Proteins for Biochemical Studies.

Peptides: Synthetic peptides containing the FP and HR2 coldspot sequences were designed and obtained (>75% purity) from GenScript (Hong Kong). Peptides were biotinylated (biotin-Ahx) at the N-terminus and amidated at the C-terminus. The aa sequence of all peptides in this study is shown in FIG. 7b.

Proteins: The CoV proteins were produced and purified as described (De Gasparo, R. et al. Nature 593, 424-428 (2021)).

S proteins: Codon-optimized gene encoding residues 1-1208 of SARS-COV-2 S ectodomain (GenBank: MN908947) was synthesized and cloned into the mammalian expression vector pcDNA3.1 (+) by Genscript; the sequence contains proline substitutions at residues 986 and 987 (S-2P), a ‘GSAS’ substitution at the furin cleavage site (residues 682-685), a C-terminal T4 fibritin trimerization motif and a C-terminal octa-histidine tag. SARS-COV-2 S ectodomains corresponding to the SARS-CoV-2 VOC were based on: Delta, GenBank: QWK65230.1; Omicron BA.1 GenBank: UFO69279.1; Omicron BA.4/BA.5 GenBank: UPP14409.1+G3V. MERS and HCoV-229E S ectodomains were based on PDB: 6NB3_A for MERS and PDB: 6U7H_A for HcoV-229E (residues 17-1142). RBD and SD1-RBD proteins: Plasmids for the production of RBD and SD1-RBD proteins were similarly designed and obtained. RBD and SD1-RBD corresponding to ancestral SARS-COV-2 were based on an early SARS-COV-2 sequence isolate (GenBank: QHO60594.1), and included aa 331-529 and 319-591, respectively. RBD and SD1-RBD corresponding to the VOC were based on: Alpha, GenBank: QWE88920.1; Beta, GenBank: QRN78347.1; Gamma, GenBank: QVE55289.1; Delta, GenBank: QWK65230.1; Omicron BA.1, GenBank: UFO69279.1; Omicron BA.2, GenBank: UJE45220.1; Omicron BA.2.75, GenBank: UTM82166.1; Omicron BA.2.75.2, GenBank: UTM82166.1+R343T+F483S; Omicron BA.4/BA.5, GenBank: UPP14409.1; Omicron BA.4.6, GenBank: UPP14409.1+R341T; Omicron BA.4.7, GenBank: UPP14409.1+R341S; Omicron BA.5.9, GenBank: UPP14409.1+R3411. SD1-RBD T572I and E583D were based on the ancestral SARS-COV-2 sequence (GenBank: QHO60594.1) with T572I or E583D, respectively. For flow cytometry-based sorting experiments, ancestral SARS-COV-2 RBD and SD1-RBD constructs were produced that included at the C-terminus an Avi-tag (GLNDIFEAQKIEWHE) for site-directed biotinylation in addition to octa-histidine tag for purification. ACE2 protein (human ACE2 fused at the C-terminus with the Fc of mouse IgG) was as previously (De Gasparo, R. et al. Nature 593, 424-428 (2021)), with synthetic, codon-optimized nucleotide sequence of hACE2 (residues 18-740) fused at the C-terminus to the Fc region of human IgG1 and cloned into pcDNA3.1 (+) vector by Genscript.All proteins were produced by transient PEI transfection in Expi293F cells (ThermoFisher), purified from the cell supernatants with proper affinity columns and analyzed to ensure functionality, stability, lack of aggregation and batch-to-batch reproducibility as previously described (De Gasparo, R. et al. Nature 593, 424-428 (2021)).

ELISAs. To evaluate the ability of antibodies to bind to peptides and proteins of CoVs, we performed enzyme-linked immunosorbent assays (ELISA).

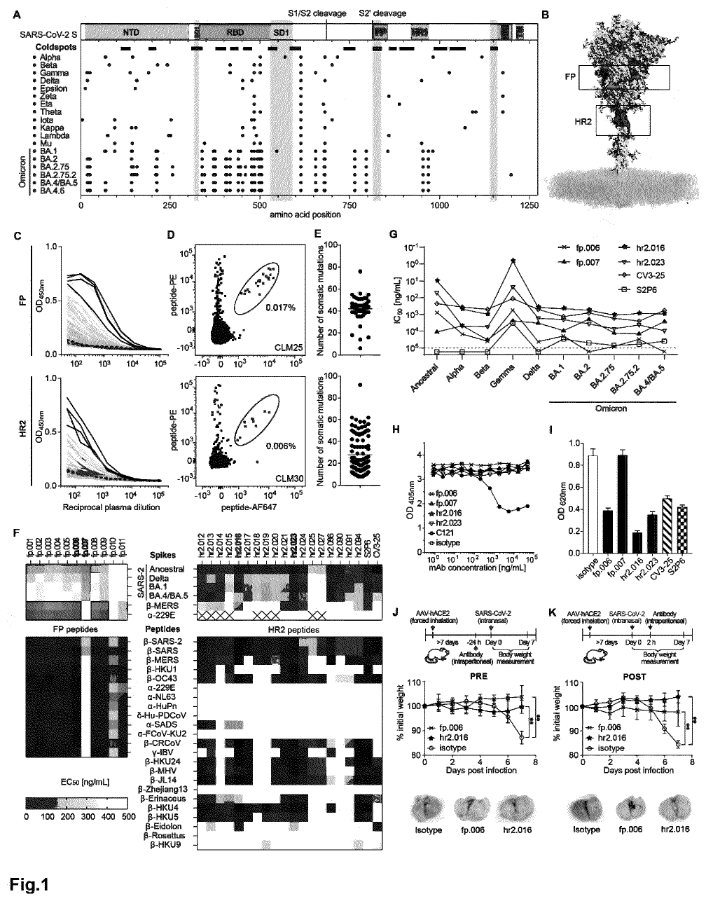

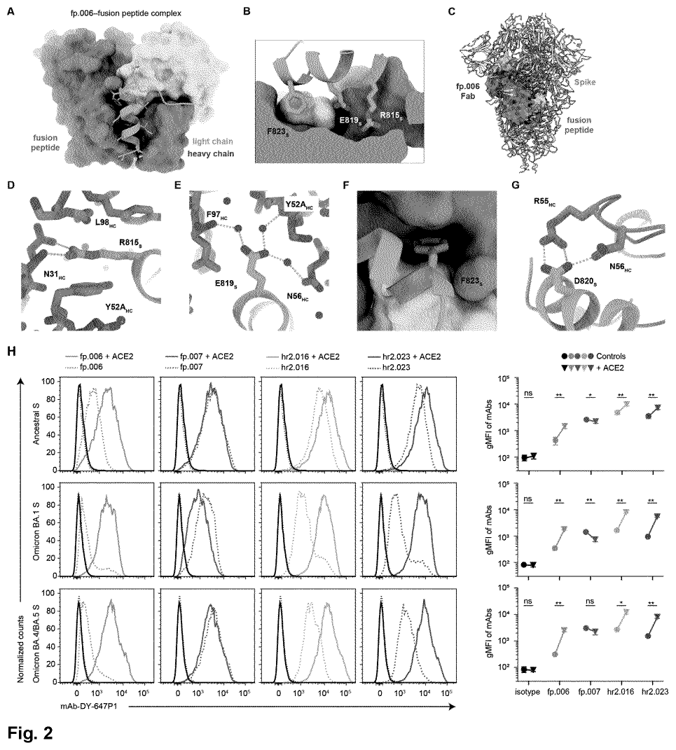

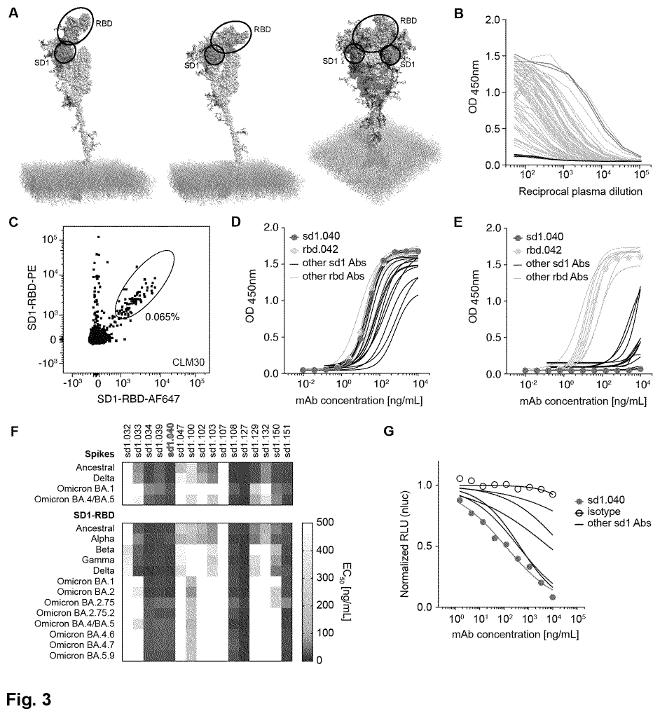

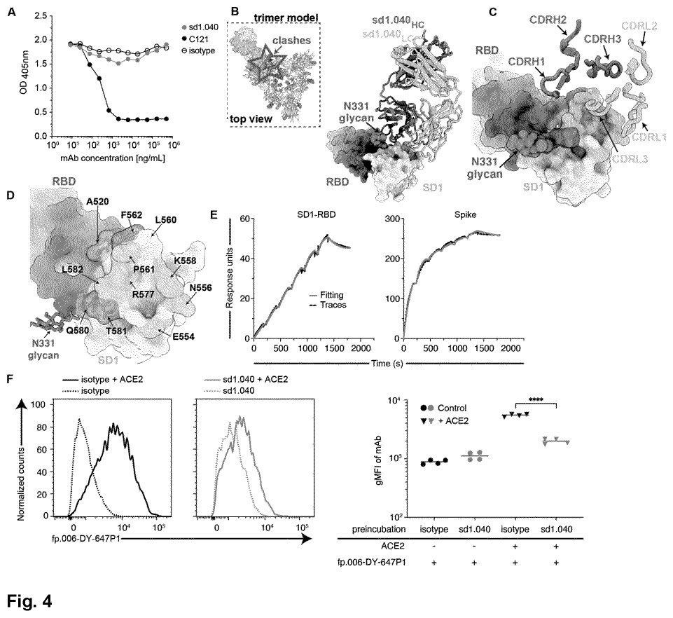

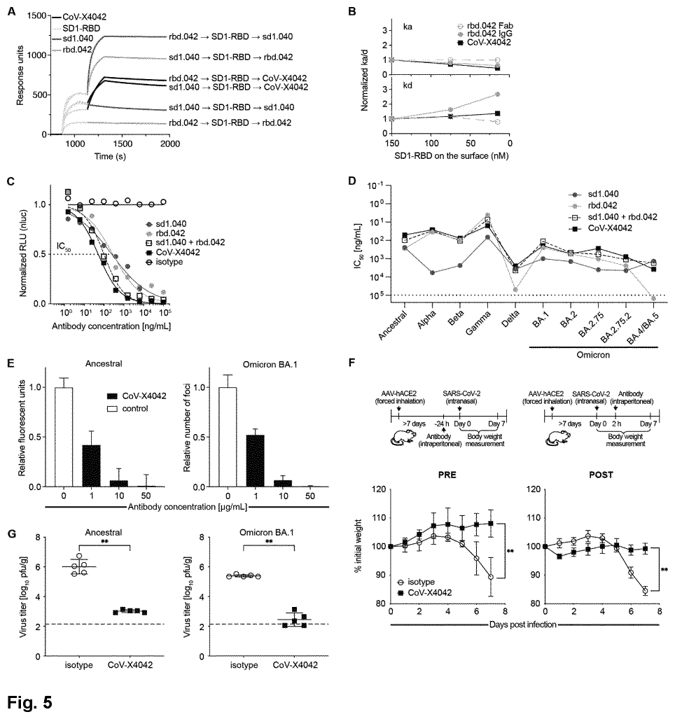

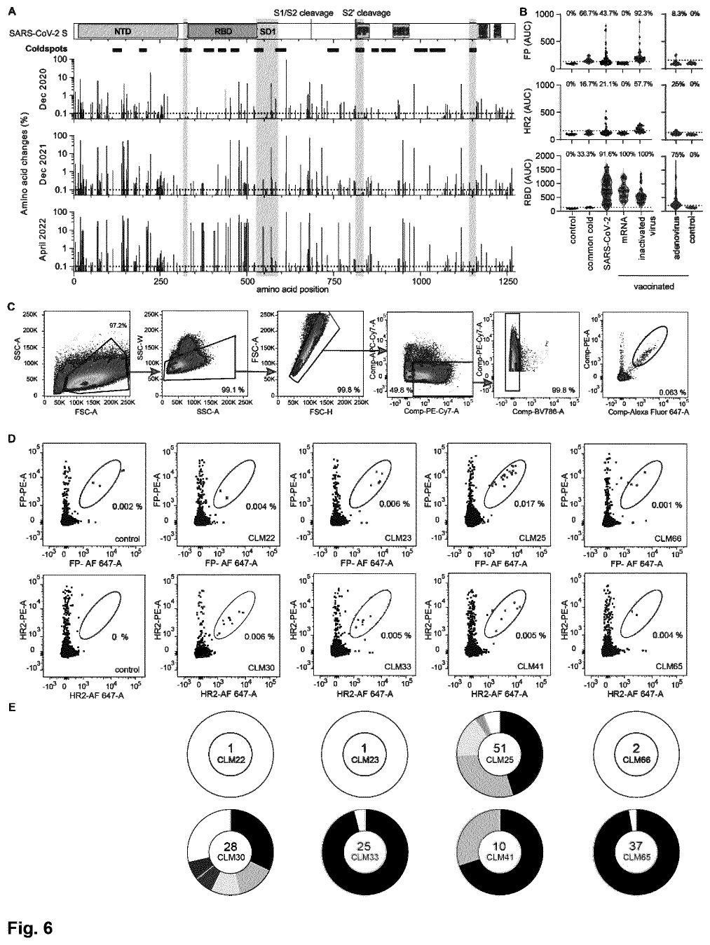

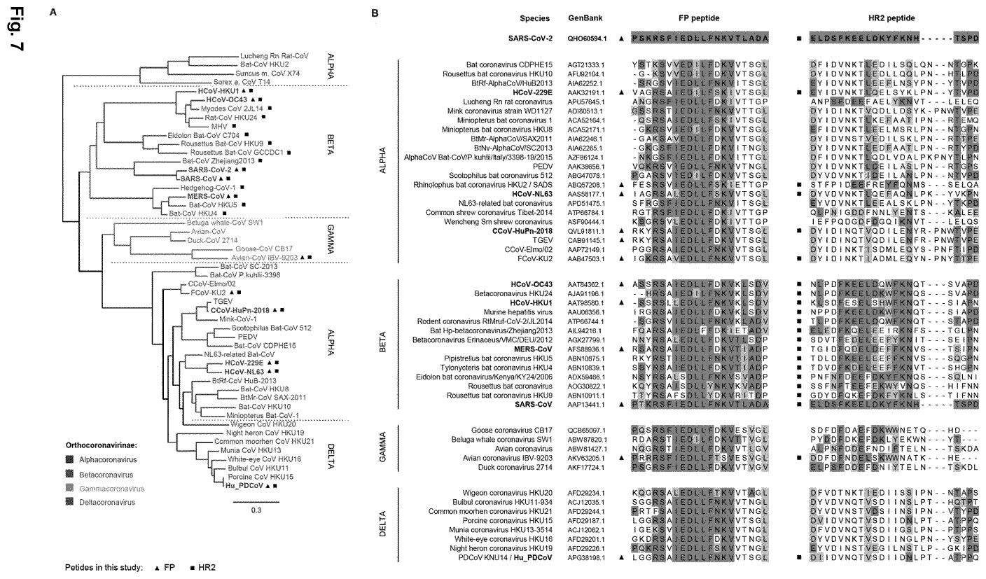

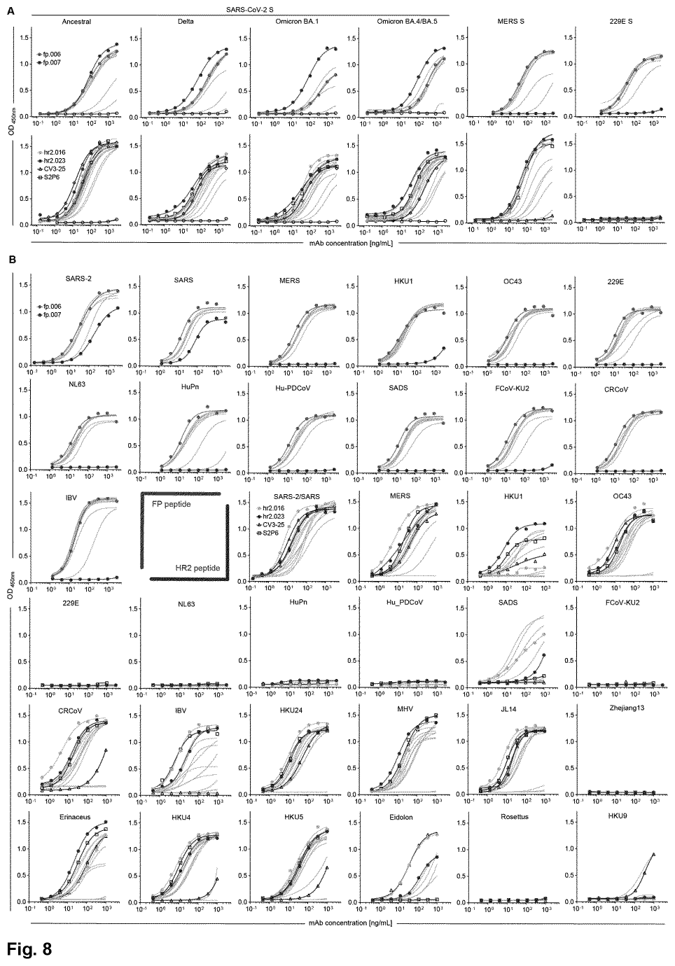

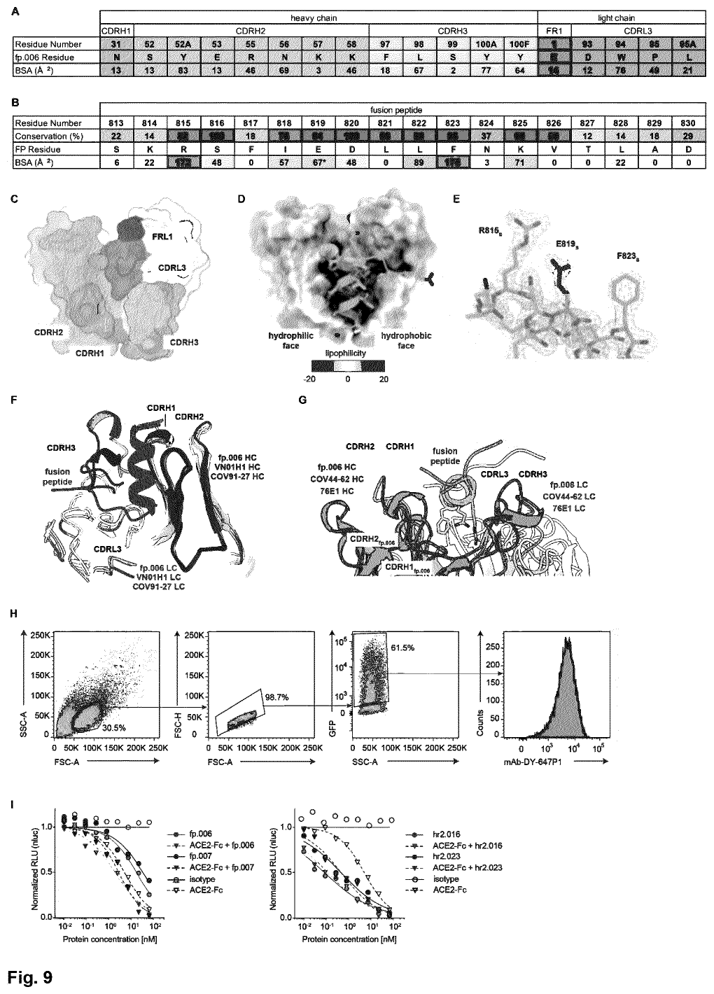

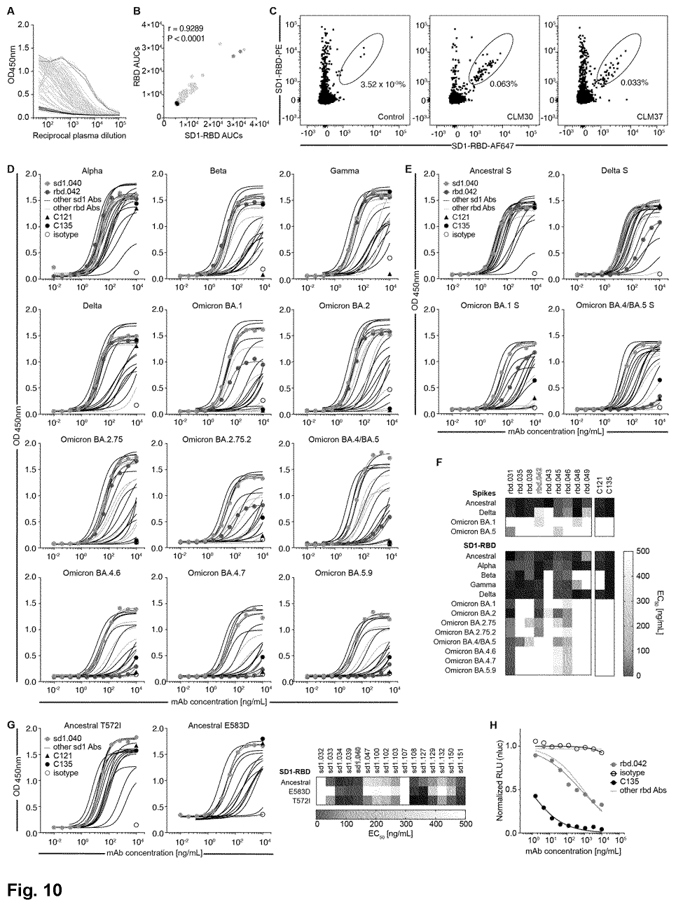

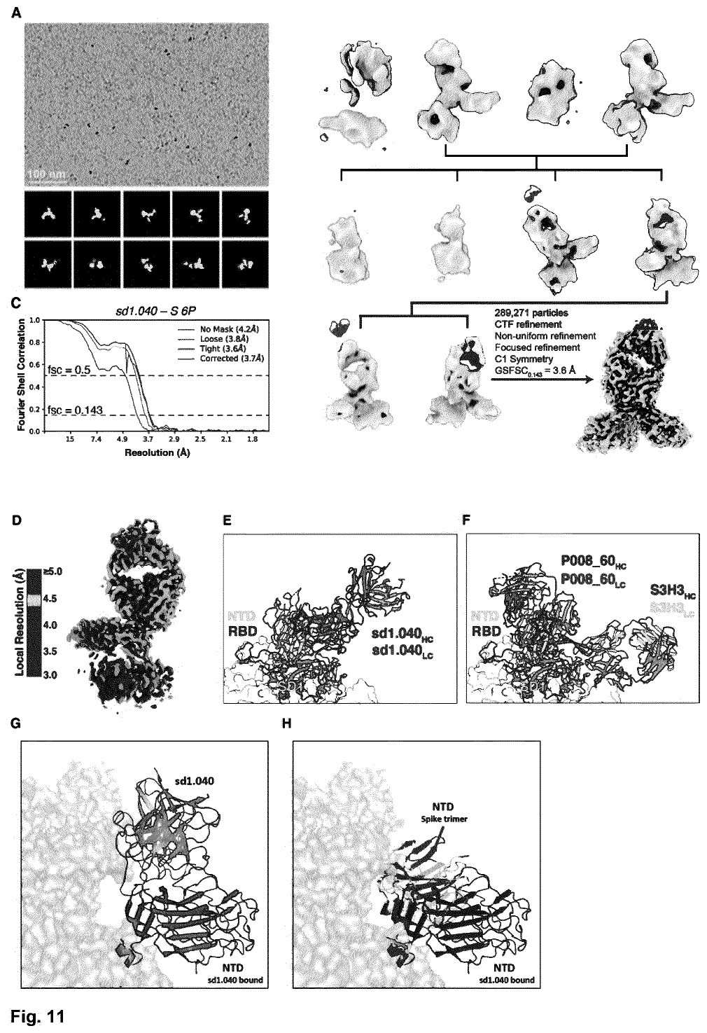

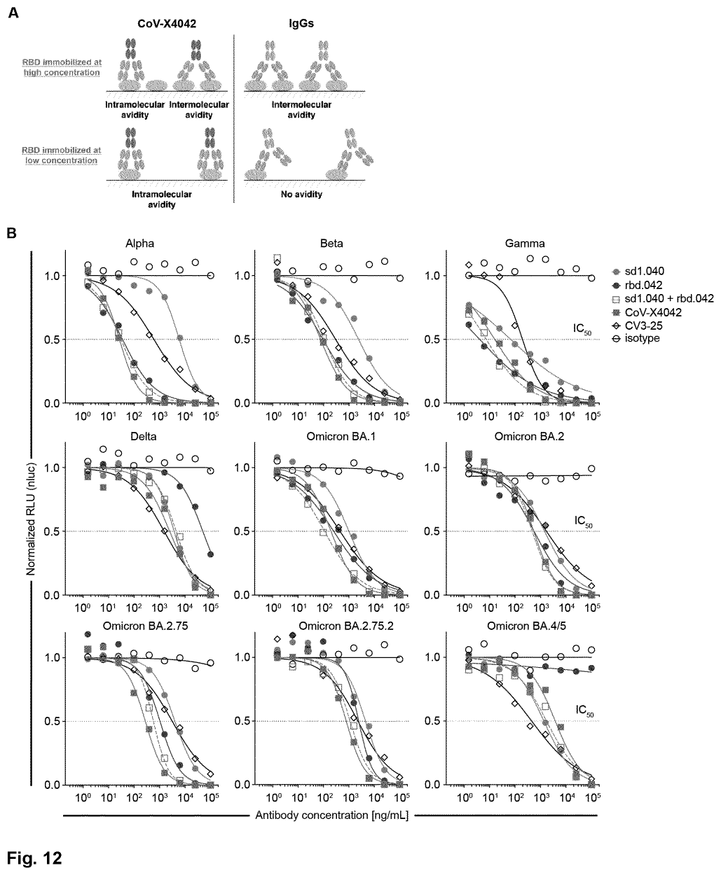

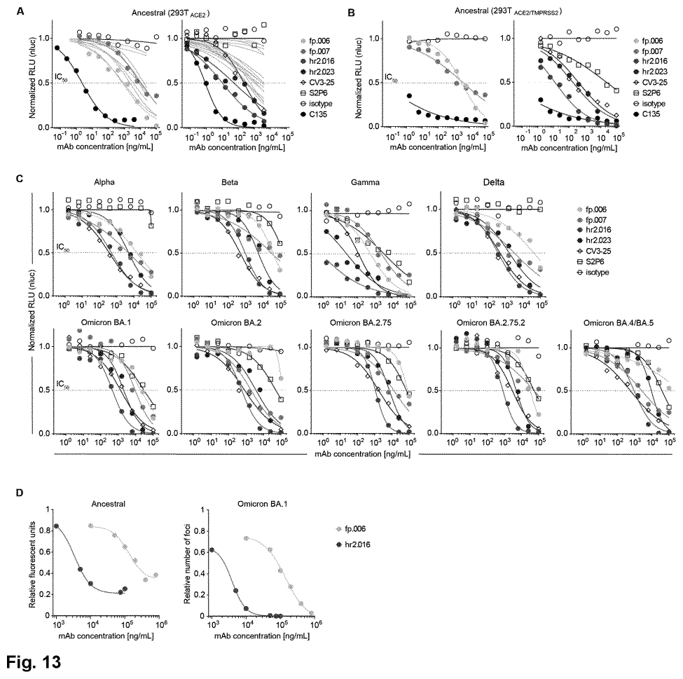

Peptide ELISA: 96-well plates (ThermoFisher, 442404) were coated with 50 μl per well of a 2 μg/ml Neutravidin (Life Technologies, 31000) solution in PBS, overnight at room temperature. Plates were washed 4 times with washing buffer (PBS+0.05% Tween-20 [Sigma-Aldrich]) and incubated with 50 μl per well of a 50 nM biotinylated peptide solution in PBS for 1 h at room temperature. After washing 4 times with washing buffer, plates were incubated with 200 μl per well of blocking buffer (PBS+2% BSA+0.05% Tween-20) for 2 h at room temperature. Plates were then washed 4 times with washing buffer, and serial dilutions of monoclonal antibodies or plasma were added in PBS+0.05% Tween-20 and incubated for 1 h at room temperature. To screen for the presence of anti-coldspot peptide IgGs, plasma samples were assayed at 1:50 starting dilution followed by 7 (FIGS. 1C and 3B) or 3 (FIG. 6) threefold serial dilutions. Monoclonal antibodies were tested starting at the indicated concentrations and followed by threefold serial dilutions. Plates were subsequently washed 4 times with washing buffer and incubated with anti-human IgG secondary antibody conjugated to horseradish peroxidase (HRP) (GE Healthcare, NA933) at a 1:5000 dilution in PBS+0.05% Tween-20. Finally, after washing 4 times with washing buffer, plates were developed by the addition of 50 μl per well of the HRP substrate TMB (ThermoFisher, 34021) for 10 min. The developing reaction was stopped with 50 μl per well of a 1M H₂SO₄ solution, and absorbance was measured at 450 nm with an ELISA microplate reader (BioTek) with Gen5 software. The Area Under the Curve (AUC) was obtained from two independent experiments and plotted with GraphPad Prism.

Protein ELISA: Experiments were performed with 96-well plates coated with 50 μl per well of a 5 μg/ml protein solution in PBS overnight at room temperature, and subsequently blocked and treated as described above. Monoclonal antibodies were tested starting at the indicated concentrations and followed by three-, four- or fivefold serial dilutions. Cross-reactivity ELISAs on SD1-RBD variants were performed in 96-well plates with half-area (Corning, 3690) and using half of the volumes mentioned above.

ACE2 binding ELISA: Experiments were as previously described (De Gasparo, R. et al. Nature 593, 424-428 (2021)). Briefly, 96-well plates with half-area (Corning, 3690) were coated with 25 μl per well of a 5 μg/mL spike solution in PBS and incubated overnight at 4° C. After washing, blocking was performed with 10% FBS in PBS for 1 h at RT. Monoclonal antibodies were added at the indicated concentrations and followed by threefold serial dilutions in blocking buffer. After washing, 25 μl per well of a 5 μg/mL solution of human ACE2 fused to the Fc portion of mouse IgG were added to the plate. Detection of ACE2 was performed with an AP-conjugated anti-mouse IgG secondary Ab (Southern Biotechnology Associates, 1030-04) diluted 1:500 in blocking buffer.

Protein biotinylation for use in flow cytometry. Purified, Avi-tagged SARS-COV-2 RBD and SD1-RBD (both corresponding to SARS-COV-2 ancestral virus) were biotinylated using the Biotin-Protein Ligase-BIRA kit according to manufacturer's instructions (Avidity). Ovalbumin (Sigma, A5503-1G) was biotinylated using the EZ-Link Sulfo-NHS-LC-Biotinylation kit according to the manufacturer's instructions (Thermo Scientific). Biotinylated Ovalbumin and RBD were conjugated to streptavidin-BV711 (BD biosciences, 563262) and SD1-RBD to streptavidin-PE (BD biosciences, 554061) and streptavidin-Alexa Fluor 647 (AF647, Biolegend, 405237), respectively.

Single-cell sorting by flow cytometry. B cells from PBMCs of uninfected controls or of COVID-19 convalescent individuals were enriched using the pan-B-cell isolation kit according to manufacturer's instructions (Miltenyi Biotec, 130-101-638). The enriched B cells were subsequently stained in FACS buffer (PBS+2% FCS+1 mM EDTA) with the following antibodies/reagents (all at 1:200 dilution) for 30 min on ice: anti-CD20-PE-Cy7 (BD Biosciences, 335828), anti-CD14-APC-eFluor 780 (Thermo Fischer Scientific, 47-0149-42), anti-CD16-APC-eFluor 780 (Thermo Fischer Scientific, 47-0168-41), anti-CD3-APC-eFluor 780 (Thermo Fischer Scientific, 47-0037-41), anti-CD8-APC-eFluor 780 (Invitrogen, 47-0086-42), Zombie NIR (BioLegend, 423105), as well as fluorophore-labeled ovalbumin (Ova) and peptides. Live single Zombie-NIR⁻CD14⁻CD16⁻CD3⁻CD8⁻CD20⁺Ova⁻peptide⁻PE⁺peptide-AF647⁺ B cells were single-cell sorted into 96-well plates containing 4 μl of lysis buffer (0.5×PBS, 10 mM DTT, 3,000 units/ml RNasin Ribonuclease Inhibitors [Promega, N2615]) per well using a FACS Aria III, and the analysis was performed with FlowJo software. The isolation of SD1-enriched B cells was performed similarly, except that sorted cells were live single Zombie-NIR⁻CD14⁺CD16⁺CD3⁺CD8⁺CD20⁺Ova⁻RBD⁻SD1-RBD-PE⁺SD1-RBD-AF647⁺. The gating strategy is shown in FIG. 6.

Antibody gene sequencing, cloning and expression. Antibody gene sequences were identified as described previously (Robbiani, D. F. et al. Nature 584, 437-442 (2020). Briefly, single cell RNA was reverse-transcribed (SuperScript III Reverse Transcriptase, Invitrogen, 18080-044) and the cDNA stored at −20° C. or used for subsequent amplification of the variable IGH, IGL and IGK genes by nested PCR and Sanger sequencing. Amplicons from the first PCR reaction were used as templates for Sequence- and Ligation-Independent Cloning (SLIC) into antibody expression vectors. Recombinant monoclonal antibodies and Fabs were produced and purified as previously described (Robbiani, D. F. et al. Cell 169, 597-609.e11 (2017)). C121, C135 anti-SARS-COV-2 antibodies and isotype control anti-Zika virus antibody Z021 were previously published (Robbiani, D. F. et al. Nature 584, 437-442 (2020); Robbiani, D. F. et al. Cell 169, 597-609.e11 (2017)); the sequences of antibodies CV3-25 and S2P6 were derived from the literature (GenBank: MW681575.1 and MW681603.1 (M. F. Jennewein et al., Cell Rep. 36, 109353 (2021)); PDB 7RNJ (D. Pinto et al, Science. 373, 1109-1116 (2021)) and produced in house starting from synthetic DNA (Genscript). The human IgG-like bispecific CoV-X4042 was designed based on the variable regions of antibodies sd1.040 and rbd.042 in the CrossMAb format (W. Schaefer, J. T. et al., Proc. Natl. Acad. Sci. U.S.A 108, 11187-11192 (2011). Four pcDNA3.1(+) mammalian expression plasmids for CrossMAb production were synthesized (Genscript), used to transfect Expi293F cells (ThermoFisher) in a 1:1:1:1 ratio, and purified from the cell supernatants as previously described (R. De Gasparo et al., Nature. 593, 424-428 (2021)). All the antibodies were tested to ensure functionality, stability and batch-to-batch reproducibility.

SARS-COV-2 pseudotyped reporter viruses. The generation of plasmids to express a C-terminally truncated SARS-COV-2 S protein (pSARS-COV2-S_{trunc}), the HIV-1 structural/regulatory proteins (pHIV_{NL}GagPol) and the NanoLuc reporter construct (pCCNanoLuc2AEGFP) were previously described (Schmidt, F. et al. J. Exp. Med. 217, e20201181 (2020)), and like the pSARS-COV2-S_{trunc} plasmid for Delta variant, they were kindly gifted by Drs. Paul Bieniasz and Theodora Hatziioannou (The Rockefeller University, New York). Plasmids expressing Alpha, Beta, Gamma, Delta, Omicron BA.1, BA.2, BA.2.75, BA.2.75.2 and BA.4/BA.5. SARS-COV-2-S_{trunc} variants were generated in house by site-directed mutagenesis (QuikChange Multi Site-Directed Mutagenesis Kit, Agilent) starting from synthetic DNA (Genscript). The sequences corresponding to SARS-COV-2 VOC were based on: Alpha (B1.1.7; GenBank QWE88920.1), Beta (B.1.351; GenBank QRN78347.1), Gamma (B.1.1.28.1; GenBank QRX39425.1), Delta (B.1.617.2; GenBank QWK65230), Omicron BA.1 (B.1.1.529; GenBank UFO69279.1), Omicron BA.2 (GenBank ULB15050.1), Omicron BA.2.75 (GenBank UTM82166.1), Omicron BA.2.75.2 (GenBank UTM82166.1+R343T+F483S+D1196N), Omicron BA.4/BA.5 (GenBank UPP14409.1+G3V). In all pseudoviruses, the intracellular domain was similarly truncated and the S1/S2 furin cleavage site was unchanged. The generation of pseudotyped virus stocks was as previously described, with minor modifications (Schmidt, F. et al. J. Exp. Med. 217, e20201181 (2020)). Briefly, 293T cells were transfected with pHIV_{NL}GagPol, pCCNanoLuc2AEGFP and pSARS-COV2-S_{trunc} plasmids using PEI-MAX (Polysciences). At 24 h after transfection, supernatants containing non-replicating virions were harvested, filtered and stored at −80° C. Infectivity was determined by titration on 293T_{ACE2} and 293_{TACE2/TMPRSS2} cells.

Pseudotyped virus neutralization assay. The assay was previously described (Schmidt, F. et al. J. Exp. Med. 217, e20201181 (2020)). Briefly, three- or four-fold serially diluted monoclonal antibodies were incubated with the SARS-COV-2 pseudotyped virus for 1 hour at 37° C. degrees. The mixture was subsequently incubated with 293TACE2 or 293TACE2/TMPRSS2 cells for 48 hours, after which cells were washed once with PBS and lysed with Luciferase Cell Culture Lysis 5x reagent (Promega). Nanoluc Luciferase activity of lysates was then measured using the Nano-Glo Luciferase Assay System (Promega) with GloMax Discover System reader (Promega). Relative luminescence units were then normalized to those derived from cells infected with SARS-COV-2 pseudotyped virus in the absence of monoclonal antibodies. The half-maximal inhibitory concentration of monoclonal antibodies (IC50) was determined using four-parameter nonlinear regression curve fit (GraphPad Prism).

Detection of monoclonal antibody binding to S by flow cytometry. 1.9×10⁶ 293T cells were plated in 60×15 mm dishes (Corning, Ref #430166) and co-transfected with two plasmids encoding GFP (2.25ug) and the SARS-COV-2 S protein (pSARS-COV2-S_{trunc}; 2.25ug) using 18 μg PEI-MAX as a transfection reagent 24 hours later. 40 hours upon transfection, cells were collected by gentle pipetting, and 50′000 transfected cells per well (in U-bottom 96-well plates; Corning, Ref #3799) were subsequently stained with 10 μg/ml pre-labelled monoclonal antibodies in the presence or absence of 30 μg/ml human ACE2 in a total volume of 100 μl PBS supplemented with 5% FBS and 2 mM EDTA for 2 h at room temperature similar to a previous report (Low, J. S. et al. bioRxiv 2022.03.30.486377 (2022)). (FIG. 2H). In FIG. 4F, cells were pre-incubated with sd1.040 or Z021 (isotype) antibodies at final concentration of 10 μg/ml for 30 minutes at room temperature before the addition of ACE2 and pre-labelled fp.006. Fluorescent labeling of monoclonal antibodies was performed with the DY-647P1-NHS-ester reagent (Dyomics, Ref #647P1-01) according to manufacturer's instructions. After staining, cells were washed twice, acquired with BD FACSCanto and analyzed with FlowJo software.

Inhibition of cell-cell fusion. Inhibition of spike-mediated cell-cell fusion was tested using an assay developed by Invivogen. Briefly, hMyD88 expressing 293 cells (Invivogen, cat. code 293-hmyd) were transfected with Wuhan pSARS-COV2-S_{trunc} plasmid using jetOptimus® (Polyplus). At 24 h after transfection, cells were resuspended in complete media and incubated with 200 μg/mL of antibodies for 1 h at 37° C., before addition of SEAP reporter 293 cells expressing hACE2 (Invivogen, cat. code hkb-hace2). Cells were co-cultured for 24 h and cell-cell fusion was assessed measuring secreted embryonic alkaline phosphatase (SEAP) activity into cells supernatant using QUANTI-Blue™ n (Invivogen), according to the manufacturer's protocol.

Surface Plasmon Resonance (SPR) assays. The IgG antibody or Fab binding properties were analyzed at 25° C. using a Biacore 8K instrument (GE Healthcare) with 10 mM HEPES pH 7.4, 150 mM NaCl, 3 mM EDTA and 0.005% Tween-20 as running buffer. SARS-COV-2 antigens (SD1-RBD or S-2P) were immobilized on the surface of CM5 chips (Cytiva) through standard amine coupling. Increasing concentrations of IgG/Fab were injected using a single-cycle kinetics setting and dissociation was followed for 10 minutes. Analyte responses were corrected for unspecific binding and buffer responses. Curve fitting and data analysis were performed with Biacore Insight Evaluation Software v.2.0.15.12933. Competition experiments were performed to obtain information on the IgG/Fab binding regions. First, antibody was immobilized on the surface of CM5 chips (Cytiva) through standard amine coupling; SD1-RBD was then flowed to form SD1-RBD/antibody complex and, shortly thereafter, the second antibody was injected. If a binding event is detected at the final step, then the 2nd antibody has a different epitope compared to the 1st (immobilized) antibody. If no binding event is detected, the two antibodies share overlapping epitopes. Competition experiments were also used to confirm the functionality of both arms of the CoV-X4042 bispecific. First, sd1.040 or rbd.042 antibodies were immobilized on the surface of CM5 chips (Cytiva) through standard amine coupling; then SD1-RBD was flowed to form RBD-SD1/antibody complex and, shortly thereafter, CoV-X4042 was injected. The analysis and comparison of kinetics parameters at different SD1-RBD concentrations were also performed as previously described (De Gasparo, R. et al. Nature 593, 424-428 (2021)) to assess the ability of CoV-X4042 to bind bivalently to a single SD1-RBD molecule.

Cell lines. 293TACE2/TMPRSS2 cell line was generated by transfecting 293TACE2 (Schmidt, F. et al. J. Exp. Med. 217, e20201181 (2020)) cells with pCMV3-FLAG-TMPRSS2 (SinoBiological) using Lipofectamine 3000 (Invitrogen) and selected with 200 μg/ml Hygromycin B (Invivogen) two days post-transfection. 293T cells for pseudotyped virus production were cultured in DMEM supplemented with 10% FBS. 293TACE2 cells were cultured in DMEM supplemented with 10% FBS, 1% NEAA, 1 mM Sodium Pyruvate, 1x Penicillin/Streptomycin and 5 μg/mL Blasticidin. Vero cells were from ATCC (CCL-81), Expi293F and 293 FT from ThermoFisher (#A14528 and R70007). hMyD88 expressing 293 cells (Invivogen) were cultured in DMEM supplemented with 10% FBS, 1x Penicillin/Streptomycin and 10 μg/mL Puromycin. SEAP reporter 293 cells expressing hACE2 (Invivogen) were grown in DMEM supplemented with 10% FBS, 1x Penicillin/Streptomycin, 1 μg/mL Puromycin and 100 μg/mL Zeocin.

Focus Reduction Neutralization Tests (FRNT). The assay was performed similar to how previously described (Robbiani, D. F. et al. Nature 584, 437-442 (2020)). Briefly, the day before infection, Vero cells were seeded at 1×10⁴ cells/well in 96-well plates.

The antibodies were diluted to final concentrations in Dulbecco's modified Eagle's medium (DMEM) supplemented with 10% newborn calf serum, 100 U/mL penicillin, 100 μg/mL streptomycin, and 1% glutamine (Sigma-Aldrich, Prague, Czech Republic). Subsequently, the diluted samples were mixed with 1000 PFU/well of ancestral SARS-CoV-2 (strain SARS-COV-2/human/Czech Republic/951/2020) or Omicron (B.1.1.529-like; hCoV-19/Czech Republic/KNL_2021-110119140/2021) and incubated at 37° C. for 90 minutes. The antibody-virus mixture was then applied directly to Vero cells (MOI of ˜0.1 PFU/cell) and incubated for 22 hours at 37° C. and 5% CO₂. Cells were then fixed by cold acetone-methanol fixation (1:1, v/v) and blocked with 10% fetal bovine serum. Cells were incubated with a rabbit (2019-nCOV) S1 antibody (1:50; Sino Biological, Duesseldorf, Germany) and then incubated for 1 hour at 37° C. with secondary goat anti-rabbit antibodies conjugated with fluorescein isothiocyanate (FITC; 1:250; Sigma-Aldrich, Prague, Czech Republic). Fluorescence intensity was measured using the Synergy H1 microplate reader (BioTek) with the following parameters: Plate type (96 WELL PLATE), fluorescence (area scan) excitation 490/emission 525, optics (Top) and gain (125), light source (Xenon Flash), lamp energy (High), reading speed (Normal), delay (100 msec), and reading height (6 mm). For Omicron, fluorescent foci were manually counted using an Olympus IX71 epifluorescence microscope and the numbers obtained normalized to no antibody control.

In vivo protection experiments. This study was performed in strict accordance with Czech laws and guidelines on the use of experimental animals and the protection of animals against cruelty (Animal Welfare Act No. 246/1992 Coll.). The protocol was approved by the Ethics Committee for Animal Experiments of the Institute of Parasitology, Institute of Molecular Genetics of the Czech Academy of Sciences, and by the Departmental Expert Committee for Approval of Projects of Experiments on Animals of the Czech Academy of Sciences (approvals 82/2020 and 101/2020). Thirteen-to fifteen-week-old female C57BL/6NCrl mice were ACE2-humanized by inhalation of a modified adeno-associated virus (AAV) (AAV-hACE2), as described previously (De Gasparo, R. et al. Nature 593, 424-428 (2021)). At least 7 days after application of AAV-hACE2 virus particles, mice were intranasally infected with SARS-CoV-2 (1×10⁴ plaque-forming units; ancestral strain SARS-COV-2/human/Czech Republic/951/2020, or Omicron B.1.1.529-like; hCoV-19/Czech Republic/KNL_2021-110119140/2021, both isolated from clinical specimens at the National Institute of Public Health, Prague; passaged five times (six times for Omicron) in Vero E6 cells before use in this study) in a total volume of 50 μl DMEM. Twenty-four hours before (pre-exposure prophylaxis), or 2 hours after (post-exposure prophylaxis) infection, mice were injected intraperitoneally with either hr2.016, CoV-X4042 (both at 300 μg), fp.006 (500 μg in pre-exposure or 300 μg in post-exposure) or isotype control (either at 300 or 500 μg). Mice were culled at the indicated time points after infection, and their tissues were collected for analysis. Lung tissue was homogenized using Mixer Mill MM400 (Retsch, Haan, Germany) and processed as a 20% (w/v) suspension in DMEM containing 10% newborn calf serum. Homogenates were clarified by centrifugation at 14,000 g (10 min, 4° C.), and supernatant medium was used for plaque assay as previously described (De Gasparo, R. et al. Nature 593, 424-428 (2021)).

Computational analyses of viral sequences. Sequences of reference for ancestral SARS-COV-2 and its variants of interest and concern (FIG. 1A) were derived from Viralzone (https://viralzone.expasy.org/9556), matched the WHO classification (https://www.who.int/en/activities/tracking-SARS-COV-2-variants/) and were as follow: ancestral SARS-COV-2 Wuhan-Hu-1 (19A; GenBank: QHO60594.1); Alpha (B1.1.7; GenBank: QWE88920.1); Beta (B.1.351; GenBank: QRN78347.1); Gamma (B.1.1.28.1; GenBank: QVE55289.1); Delta (B.1.617.2; GenBank: QWK65230.1); Epsilon (B.1.427; B.1.429; GenBank QQM19141.1); Zeta (B.1.1.28.2; GenBank; QQX30509.1); Eta (B.1.525; GenBank: QRF70806.1); Theta (B.1.1.28.3); lota (B.1.526; GenBank: QRX49325.1); Kappa (B.1.617.1; GenBank: QTY83052.1); Lambda (B.1.1.1.C37; GenBank: QTJ90974.1); Mu (B.1.621); Omicron BA.1 (previously B.1.1.529; GenBank: UFO69279.1); Omicron BA.2 (GenBank: ULB15050.1); Omicron BA.3 (GISAID: EPI ISL 9092427); Omicron BA.4 (GenBank: Upp14409.1); omicron BA.5 (GenBank: UOZ45804.1). For the analysis of SARS-COV-2 amino acid substitutions, viral sequences of S that were present in GISAID as of either 31 Dec. 2020, 31 Dec. 2021, or Apr. 29 2022, were downloaded. Sequences with a length of S corresponding between 1223 and 1323 aa, and no undetermined aa, were aligned to determine the frequency of aa changes over ancestral reference sequence using BLASTP version 2.5.0 with default settings. Frequencies were computed using in house developed bash and C++ pipeline available at GitHub (https://github.com/cavallilab/coldspot). The models of the full S, glycosylated and with a membrane, both closed and open conformation, were taken from a previous publication (Casalino, L. et al. ACS Cent. Sci. 6, 1722-1734 (2020)) and rendered with Pymol 2.3.5 (FIGS. 1c and 3a). For the phylogenetic analysis and peptide sequence alignment, sequences of representative S protein of CoV species classified according to the ICTV taxonomical classification (https://talk.ictvonline.org/taxonomy/) (International Committee on Taxonomy of Viruses Executive Committee. Nat. Microbiol. 5, 668-674 (2020)) were derived from the NCBI taxonomy database (https://www.ncbi.nlm.nih.gov/data-hub/taxonomy/11118/) (Schoch, C. L. et al. Database J. Biol. Databases Curation 2020, baaa062 (2020)), aligned using ClustalW (SnapGene), and the phylogenetic tree was built using phylogeny.fr with default settings (Dereeper, A. et al. Nucleic Acids Res. 36, W465-469 (2008)).

X-ray crystallography. fp.006 Fab in 1×TBS (20 mM Tris, 150 mM NaCl) was mixed with the fusion peptide (PBS with 10% DMSO; PSKRSFIEDLLFNKVTLADA with N-terminal Biotin-Ahx and C-terminal amidation) at a 1:2 molar ratio (Fab:peptide). The sample was incubated overnight at room temperature, and then concentrated to ˜8.8 mg/mL using an Amicon spin filter with a 30 kDa molecular weight cutoff (Millipore Sigma) after diluting with an additional sample volume of 1×TBS to decrease the proportion of PBS and DMSO in the complex.

Crystallization trials were set up using the sitting drop vapor diffusion method by mixing equal volumes of fp.006-FP complex and reservoir using a Douglas Oryx8 robot and commercially available 96-well crystallization screens (Hampton Research). Crystals were grown at 16° C. and observed in multiple conditions. The single crystal that was used for structure determination of fp.006-FP was obtained in 0.2 M Potassium phosphate monobasic pH 4.8 and 20% w/v Polyethylene glycol 3350, and were cryoprotected in a solution matching the reservoir and 30% glycerol and then cryocooled in liquid nitrogen.

X-ray diffraction data was collected at the Stanford Synchrotron Radiation Lightsource (SSRL) beamline 12-1 with an Eiger X 16 M pixel detector (Dectris) at a wavelength of 0.979 Å and temperature of 100 K. Data from a single crystal was indexed and integrated in XDS/Dials (Kabsch, W. Acta Crystallogr. D Biol. Crystallogr. 66, 133-144 (2010)), and then merged using AIMLESS in CCP4 (Winn, M. D. et al. Acta Crystallogr. D Biol. Crystallogr. 67, 235-242 (2011)). Structures were determined using molecular replacement in PHASER (McCoy, A. J. et al. J. Appl. Crystallogr. 40, 658-674 (2007)) using two copies of each of the following individual chains as search models: VH (PDB: 4GXU chain M with CRH3 trimmed), VL (PDB: 6FG1 chain B with CDRL3 trimmed), CH (PDB: 4GXU), and CL (PDB: 6FG1). Coordinates were refined using iterative rounds of automated and interactive refinement in Phenix (Adams, P. D. et al. PHENIX: a comprehensive Python-based system for macromolecular structure solution. Acta Crystallogr. D Biol. Crystallogr. 66, 213-221 (2010)) and Coot (Emsley, P., Lohkamp, B., Scott, W. G. & Cowtan, K. Acta Crystallogr. D Biol. Crystallogr. 66, 486-501 (2010)), respectively. The final model contains 97.9% Ramachandran favored residues, with 2.0% allowed and the remaining 0.1% Ramachandran outliers.

Cryo-EM sample preparation. Concentrated and purified sd1.040 Fab was mixed with SARS-COV-2 S-2P trimer at a 1.1:1 molar ratio (Fab: trimer) to a final concentration of 3 mg/mL and incubated at room temperature for thirty minutes. Immediately prior to application of 3.1 μL of sample to a freshly glow-discharged 300 mesh Quantifoil R1.2/1.3 grid, fluorinated octyl-maltoside (FOM) was added to a final concentration of 0.02% (w/v). Complex was vitrified by plunging into 100% liquid ethane after blotting for 3.5 s with Whatman No. 1 filter paper at 22° C. and 100% humidity using a Mark IV Vitrobot (Thermo Fisher).

Cryo-EM data collection and processing. Single particle movies were collected on a Titan Krios TEM (300 kV) using SerialEM automated data collection software (Mastronarde, D. N. J. Struct. Biol. 152, 36-51 (2005)) and a K3 camera (Gatan) behind a BioQuantum energy filter (Gatan) with a 20 eV slit size (0.85 Å/pixel). Data processing followed a similar workflow as previously described (Wang, Z. et al. Immunity S1074-7613 (22) 00174-1 (2022)). Briefly, 9,894 movies were patch motion corrected for beam-induced motion including dose-weighting within cryoSPARC v3.1 (Punjani, A., Rubinstein, J. L., Fleet, D. J. & Brubaker, M. A. Nat. Methods 14, 290-296 (2017)). CTF estimates were performed on non-doseweighted micrographs, which were subsequently curated to remove poor fits and images with thick ice. An initial set of particles were generated using automated blob picking, of which, a subset (300,000 particles) was used to generate four ab initio volumes. The entire particle stack (4,343,219 particles) were extracted 4x-binned and heterogeneously refined into the four ab initio volumes. Particles corresponding to the volume that best demonstrated features of a Fab bound to a protomer were cleaned up using 2D classification, and reextracted with 2x-binning. The resulting particle stack (1,552,774 particles) was further 3D classified in cryoSPARC (k=6). Particles corresponding to the 3D volumes with well-defined secondary structural features were pooled and homogeneously refined with C1 symmetry. Following CTF refinement and application of a mask for focus refinement on the RBD-SD1-Fab regions, the final reconstructed volume achieved a global resolution of 3.7 Å based on gold standard FSC calculations.

Cryo-EM structure modeling and refinement. Coordinates for the sd1.040-S1 protomer complex were generated by docking individual domains from reference structures (individual spike domains, PDB 6VXX-chain A; Fab heavy chain, PDB 5AZE-chain H; Fab light chain, PDB 7DOD-chain L) into cryo-EM density using UCSF Chimera (Goddard, T. D., Huang, C. C. & Ferrin, T. E. J. Struct. Biol. 157, 281-287 (2007)). Initial models were rigid body refined into cryo-EM density, followed by real space refinement with morphing in Phenix. Sequence matching was interactively performed in Coot and models were further refined in Phenix. Validation of model coordinates was performed using MolProbity (Chen, V. B. et al. Acta Crystallogr. D Biol. Crystallogr. 66, 12-21 (2010)).

Structural Analyses. CDR and somatic mutation assignments were produced by IMGT V-QUEST (Brochet, X., Lefranc, M.-P. & Giudicelli, V. Nucleic Acids Res. 36, W503-508 (2008).). Graphics describing structures were made in ChimeraX (Goddard, T. D. et al. Protein Sci. Publ. Protein Soc. 27, 14-25 (2018)). Buried surface areas were calculated using the online PDBePISA server (Krissinel, E. & Henrick, K. J. Mol. Biol. 372, 774-797 (2007)). Contacting residues are defined as those with less than 4 Å distance between atoms of different chains. Hydrogen bond assignments were made using a 3.5 Å cutoff and A-D-H angle greater than 90°. RMSD calculations were done in PyMOL (Schrödinger). Antibody residues are numbered according to the Kabat convention.

### Example 1

To determine whether antibodies to the FP and HR2 coldspots occur naturally, we evaluated plasma samples from a COVID-19 convalescent cohort by enzyme-linked immunosorbent assay (ELISA; n=67; see Methods). High levels of IgG antibodies binding to peptides at the FP and HR2 coldspots were found in convalescent individuals (FIG. 1C). In comparison, IgG levels were low to undetectable in samples from uninfected controls, in pre-pandemic samples obtained after documented common cold CoV infection, and in most COVID-19 vaccinated individuals, except for some who received inactivated virus-based vaccines (FIG. 6). To examine the molecular features of coldspot antibodies, we used flow cytometry to isolate B cells specific for FP and HR2 peptides from those individuals with high antibody levels in plasma (FIG. 1D, FIG. 6, C and D; see Methods). We obtained 55 (FP) and 100 (HR2) paired IgG heavy and light chain antibody sequences, some of which clustered in expanded clones of related antibodies (FIG. 6). The average number of V gene somatic nucleotide mutations was high: 42 for FP (range: 6-76) and 28 for HR2 antibodies (range: 8-92; FIG. 1E).

### Example 2

Twenty-nine monoclonal antibodies, including at least one representative for each of the expanded clones, were recombinantly expressed and tested in ELISA. Ten out of 11 FP antibodies bound to the peptide with half-maximal effective concentrations (EC₅₀) between 25-119 ng/ml. The EC₅₀ values of the same antibodies to S were on average 4.8-fold higher, except for antibody fp.007, where the observed EC₅₀ declined from 177 to 58 ng/ml (FIG. 1F and FIG. 7). Similarly, all 18 HR2 antibodies bound to the peptide with EC₅₀ values between 7-117 ng/ml, and with an average of 1.3-fold higher EC₅₀ to S. Several of the antibodies were broadly cross-reactive since they bound to representative S trimers of SARS-COV-2 VOC, MERS and HCoV-229E, as well as to FP and HR2 peptides corresponding to other CoVs. Noteworthy, most FP antibodies recognized CoVs of the four genera (alpha to delta, including all 9 CoVs associated with human disease), and some HR2 antibodies cross-reacted not only with beta-, but also with alpha- and gammacoronaviruses (FIG. 1F, FIGS. 7 and 8; Table 1).

### Example 3

To evaluate the antibodies' ability to neutralize SARS-COV-2, we used a previously established SARS-COV-2 pseudovirus assay (Robbiani, D. F. et al. Nature 584, 437-442 (2020)). The most potent FP antibody (fp.006) displayed a half-maximal inhibitory concentration (IC₅₀) of 737 ng/ml, while the best HR2 neutralizer (hr2.016) had an IC₅₀ of 10 ng/ml, which was better than previously reported antibodies to a different but similar region that were tested alongside (CV3-25 and S2P6; FIG. 1G) (Pinto, D. et al. Science 373, 1109-1116 (2021); Jennewein, M. F. et al. Cell Rep. 36, 109353 (2021)). Select anti-FP and anti-HR2 antibodies blocked infection regardless of TMPRSS2-expression by target cells and, consistent with the view that they antagonize post-attachment events, they did not interfere with ACE2 binding to S in ELISA but inhibited cell fusion (FIG. 1, H and I, FIG. 13, A and B). As expected, based on the absence of aa changes at coldspot regions, some FP and HR2 antibodies were effective against pseudoviruses corresponding to SARS-COV-2 VOC, against ancestral and Omicron SARS-COV-2 in vitro, and protected mice challenged with ancestral virus when administered either as pre- or post-exposure prophylaxis (FIG. 1, G, J and K; FIG. 13, C and D). Thus, natural antibodies exist that protect against SARS-CoV-2 by binding to highly conserved linear epitopes at functional regions of S.

### Example 4

To gain insight into the mechanism of broad recognition by fp.006, we obtained a 2.0 Å resolution crystal structure of its Fab in complex with the ₈₁₂PSKRSFIEDLLFNKVTLADA₈₃₁ FP peptide (FIG. 2). In the bound structure, FP residues 813-825 adopt an a-helical conformation, extending an amphipathic helix observed in prefusion S trimer structures. The helical peptide sits within a groove that is formed by fp.006 complementarity-determining region (CDR) 3 loops, which mediate most epitope contacts (FIG. 2A). Additional contacts with heavy chain CDR1 and CDR2 loops result in a total buried surface area (BSA) of 1,466 Å² (686 Å² paratope BSA+780 Å² epitope BSA) Of the 13 antigenically distinct CoV FPs tested here, the majority of epitope residues contacted by fp.006 are highly conserved, explaining fp.006's breadth of binding (FIG. 1F; FIGS. 7B and 9B). In particular, three FP residues (R₈₁₅, E₈₁₉, and F₈₂₃) are completely buried in the Fab groove and make extensive hydrogen bond and hydrophobic interactions (FIG. 2, B to G). As a result, one face of the FP amphipathic helix comprises two polar residues that contact a polar patch on the edge of the Fab trough formed by CDRH2, and the opposite hydrophobic face engages hydrophobic residues in the CDRH3 loop (FIG. 9, D and E).Notably, residue R₈₁₅, which is critical for TMPRSS2 and Cathepsin cleavage, forms hydrogen bonds with the Fab CDRH1 loop and a cation-π interaction with Y_{52A} in CDRH2 (FIG. 2D). Given the importance of R₈₁₅ in protease cleavage, fp.006-mediated neutralization likely includes steric hinderance of TMPRSS2 and Cathepsin binding and further processing of the S trimer for productive fusion. Interestingly, superposition of the Fab-FP complex crystal structure with a prefusion S trimer structure revealed an approach angle incompatible with Fab binding, which explains the weak binding observed for FP antibodies to prefusion S trimers (FIG. 1F and FIG. 2C). Thus, cleavage by cellular proteases at the S2′ site and antibody recognition of this partially cryptic epitope likely involves transient conformational changes that are necessary to expose the FP epitope.

In agreement with this view, ACE2 engagement of cell surface-expressed S, which is known to alter S conformation, increased fp.006 binding by 5.8-fold in a flow cytometry assay, and the addition of soluble ACE2 synergized with fp.006 for neutralization (FIG. 2H and FIG. 9, H and I). Interestingly, and consistent with the ELISA and neutralization data (FIG. 1, F and G), binding of fp.006 to S was weaker with Omicron by this assay, but ACE2 attachment improved it to similar levels as with ancestral (5.3-fold for BA.1 and 8.2-fold for BA.4/BA.5; FIG. 2H). Therefore, ACE2 can induce conformational changes of S that expose the highly conserved FP epitope and favor neutralization. ACE2 attachment also increased the binding of hr2.016 and hr2.023 to both ancestral (2.3- and 2.4-fold) and Omicron S (4.9-fold and 5.8-fold for BA.1, 4.6-fold and 5.3-fold for BA.4/BA.5, respectively; FIG. 2H), but did not improve the binding of fp.007, a neutralizing FP-antibody that displays a different pattern of cross-reactivity than fp.006 (FIGS. 1F and 2H). Therefore, optimal FP recognition by neutralizing fp.007-like antibodies does not require ACE2-induced conformational changes.

### Example 5

The subdomain 1 (SD1) of SARS-COV-2 S is adjacent to the RBD and its sequence is conserved across SARS-COV-2 variants, except for substitutions A570D in Alpha (B.1.1.7) and T547K in Omicron BA.1 (B.1.1.529; FIGS. 1, A and B and 3A). To identify antibodies targeting SD1, we designed a flow cytometry-based strategy that combines negative selection of B cells binding to RBD (aa 331-529) with positive selection of those binding to SD1-RBD (aa 319-591; FIG. 6; see Methods). Peripheral blood mononuclear cells were obtained from individuals with high plasma IgG reactivity to SD1-RBD (FIG. 3B and FIG. 10, A and B) and B cells enriched for binding to SD1 were sorted as single cells for antibody gene sequencing (FIG. 3C, FIG. 10C).

Twenty-five monoclonal antibodies were cloned and produced. All 25 bound to SD1-RBD in ELISA, and 16 were SD1-specific (FIG. 3, D and E). Six of the SD1 antibodies cross-reacted with SD1-RBD proteins corresponding to all twelve CoV VOC with EC₅₀ values of 66.25 ng/ml or lower, while only two RBD antibodies cross-reacted with all variants' RBD at low EC₅₀ (FIG. 3F; FIG. 10, D to F). Select antibodies also bound effectively to the common SD1 variants T572I and E583D (FIG. 10G). In pseudovirus-based neutralization assays, the best broadly cross-reactive SD1 antibody was sd1.040 (IC₅₀=245 ng/ml; FIG. 3G). This demonstrates that naturally occurring antibodies can neutralize SARS-COV-2 by binding to SD1.

### Example 6

The mechanism of neutralization by sd1.040 does not involve inhibition of receptor binding, since sd1.040, unlike C121, failed to prevent ACE2 binding to S in ELISA (FIG. 4a). To gain insight into the neutralization mechanism, we formed a complex between sd1.040 Fabs bound to a prefusion S-2P trimer and used cryo-electron microscopy (cryo-EM) for structure determination. Interestingly, no intact Fab-S trimer structures were observed within the dataset (FIG. 11A to D). Instead, our 3.7 Å cryo-EM structure revealed sd1.040 Fabs in complex with S1 protomers, recognizing an epitope comprising SD1 residues 554-562, 577-581, and RBD residues 520-524 (FIG. 4, B to D, FIG. 11A to D). Superposition of the cryo-EM sd1.040-S protomer complex on published prefusion S structures, indiscriminate of RBD conformation, revealed minor clashes with the N-terminal domain of the adjacent protomer similar to antibody P008_60 which contrasts the recently described SD1-specific murine antibody S3H3 (FIG. 4B, inset, and FIG. 11, E and F). It is likely that sd1.040 binding to S requires minor local rearrangement of the NTD. Indeed, docking and Molecular Dynamics computational simulations suggest the presence of quaternary interactions between the antibody, SD1 and RBD on one protomer, and NTD on another, which require minor rearrangement of the NTD hinging around residues 295-300 towards the end of the NTD (FIG. 11, G and H). Such rearrangement does not result in clashes with other S regions. The CDRH1 and CDRH3, which do not interact with RBD/SD1 in the cryo-EM protomer structure, are shown to directly contact the NTD in the simulations. These quaternary interactions are likely to stabilize the complex, which by surface plasmon resonance (SPR) displays ˜20 times stronger binding affinity for S (K_{D} 0.2 nM) than for SD1-RBD (K_{D} 4.2 nM; FIG. 4E). To test the hypothesis that the mechanism of neutralization by sd1.040 is through the inhibition of S molecular rearrangements, we measured fp.006 binding to S by flow cytometry. ACE2 attachment in this assay improves fp.006 binding to S, which is however blocked by treatment with sd1.040 (FIG. 4F). Thus sd1.040 interferes with conformational changes downstream of ACE2 attachment.

Based on the breadth of binding exhibited by antibodies sd1.040 and rbd.042 against SARS-COV-2 VOC, their potency of neutralization, and preliminary experiments that indicated synergistic effects from combining them (FIG. 3, F and G and FIG. 10, F and H), we produced a bispecific antibody that includes the moieties of both antibodies, named CoV-X4042. CoV-X4042 has a natural IgG format including the entire Fc region and complementary modifications in the Fc and CH1/CL regions that minimize formation of undesired byproducts. Consistent with the parental antibodies binding to distinct epitopes, both arms of CoV-X4042 can simultaneously engage the same SD1-RBD molecule (FIG. 5, A and B and FIG. 12). In pseudovirus-based neutralization assays, CoV-X4042, like the sd1.040/rbd.042 cocktail, exhibited significant synergistic activity over either of the parental antibodies alone (FIG. 5C). CoV-X4042 neutralized pseudoviruses corresponding to VOC, and remained effective even when one of the parental antibodies lost efficacy (e.g. rbd.042 against Omicron BA.4/BA.5; FIG. 5D). Efficacy of the bispecific antibody was confirmed against ancestral and Omicron SARS-COV-2 in vitro and by in vivo protection experiments showing that mice treated with CoV-X4042, either as pre- or post-exposure prophylaxis, maintained body weight and displayed diminished pathology and infectious virus titers in the lungs (FIG. 5, E to G). Therefore, bispecific antibodies composed of moieties that simultaneously target conserved neutralizing epitopes on SD1 and RBD are effective against SARS-COV-2 in preclinical models.

TABLE 1
Neutralizing and binding data of the monoclonal antibodies
a

| | | Ancestral | Alpha | Beta | Gamma | Delta |
|---|---|---|---|---|---|---|
| Participant | Antibody | Pseudovirus | Pseudovirus | Pseudovirus | Pseudovirus | Pseudovirus |
| ID | ID | IC₅₀ ng/mL | IC₅₀ ng/mL | IC₅₀ ng/mL | IC₅₀ ng/mL | IC₅₀ ng/mL |
| CLM25 | fp.001 | 2187 | NT | NT | NT | NT |
| CLM25 | fp.002 | 16064 | NT | NT | NT | NT |
| CLM25 | fp.003 | 9263 | NT | NT | NT | NT |
| CLM25 | fp.004 | 7844 | NT | NT | NT | NT |
| CLM25 | fp.005 | 9091 | NT | NT | NT | NT |
| CLM25 | fp.006 | 737.2 | 15602 | 29630 | 408.1 | 41984 |
| CLM25 | fp.007 | 11084 | 5852 | 42857 | 801.1 | 3228 |
| CLM25 | fp.008 | 3564.00 | NT | NT | NT | NT |
| CLM25 | fp.009 | 872.9 | NT | NT | NT | NT |
| CLM66 | fp.010 | ND | NT | NT | NT | NT |
| CLM66 | fp.011 | ND | NT | NT | NT | NT |
| CLM30 | hr2.012 | 1722 | NT | NT | NT | NT |
| CLM30 | hr2.013 | ND | NT | NT | NT | NT |
| CLM30 | hr2.014 | 355.1 | NT | NT | NT | NT |
| CLM30 | hr2.015 | 3828 | NT | NT | NT | NT |
| CLM30 | hr2.016 | 10.0 | 307.3 | 483.7 | 0.5992 | 383.8 |
| CLM30 | hr2.017 | 542.1 | NT | NT | NT | NT |
| CLM41 | hr2.018 | ND | NT | NT | NT | NT |
| CLM41 | hr2.019 | 1222 | NT | NT | NT | NT |
| CLM41 | hr2.020 | ND | NT | NT | NT | NT |
| CLM33 | hr2.021 | 3020 | NT | NT | NT | NT |
| CLM33 | hr2.023 | 52.36 | 4713 | 5569 | 20.94 | 1687 |
| CLM33 | hr2.024 | 301.4 | NT | NT | NT | NT |
| CLM65 | hr2.025 | ND | NT | NT | NT | NT |
| CLM65 | hr2.027 | ND | NT | NT | NT | NT |
| CLM30 | hr2.086 | ND | NT | NT | NT | NT |
| CLM30 | hr2.090 | 1021.00 | NT | NT | NT | NT |
| CLM30 | hr2.091 | 306.2 | NT | NT | NT | NT |
| CLM30 | hr2.094 | 2954 | NT | NT | NT | NT |
| | S2P6 | ND | ND | ND | 471.1 | ND |
| | CV3-25 | 223.3 | 467.5 | 363.9 | 147.4 | 496.9 |

b

| | | Omicron | Omicron | Omicron | Omicron | Omicron | Ancestal |
|---|---|---|---|---|---|---|---|
| | | BA.1 | BA.2 | BA.2.75 | BA.2.75.2 | BA.4/BA.5 | Pseudovirus |
| Participant | Antibody | Pseudovirus | Pseudovirus | Pseudovirus | Pseudovirus | Pseudovirus | IC₅₀ ng/mL |
| ID | ID | IC₅₀ ng/mL | IC50 ng/mL | IC50 ng/mL | IC50 ng/mL | IC₅₀ ng/mL | (on cells with |
| CLM25 | fp.001 | NT | NT | NT | NT | NT | NT |
| CLM25 | fp.002 | NT | NT | NT | NT | NT | NT |
| CLM25 | fp.003 | NT | NT | NT | NT | NT | NT |
| CLM25 | fp.004 | NT | NT | NT | NT | NT | NT |
| CLM25 | fp.005 | NT | NT | NT | NT | NT | NT |
| CLM25 | fp.006 | 19145 | ND | 80779 | 30308 | ND | 3144 |
| CLM25 | fp.007 | 9233 | 1889 | 12675 | 14469 | 2624 | 3978 |
| CLM25 | fp.008 | NT | NT | NT | NT | NT | NT |
| CLM25 | fp.009 | NT | NT | NT | NT | NT | NT |
| CLM66 | fp.010 | NT | NT | NT | NT | NT | NT |
| CLM66 | fp.011 | NT | NT | NT | NT | NT | NT |
| CLM30 | hr2.012 | NT | NT | NT | NT | NT | NT |
| CLM30 | hr2.013 | NT | NT | NT | NT | NT | NT |
| CLM30 | hr2.014 | NT | NT | NT | NT | NT | NT |
| CLM30 | hr2.015 | NT | NT | NT | NT | NT | NT |
| CLM30 | hr2.016 | 453.6 | 558.6 | 1082.0 | 867.0 | 934.0 | 12.0 |
| CLM30 | hr2.017 | NT | NT | NT | NT | NT | NT |
| CLM41 | hr2.018 | NT | NT | NT | NT | NT | NT |
| CLM41 | hr2.019 | NT | NT | NT | NT | NT | NT |
| CLM41 | hr2.020 | NT | NT | NT | NT | NT | NT |
| CLM33 | hr2.021 | NT | NT | NT | NT | NT | NT |
| CLM33 | hr2.023 | 2030 | 3600 | 7058 | 4244 | 9405 | 167.8 |
| CLM33 | hr2.024 | NT | NT | NT | NT | NT | NT |
| CLM65 | hr2.025 | NT | NT | NT | NT | NT | NT |
| CLM65 | hr2.027 | NT | NT | NT | NT | NT | NT |
| CLM30 | hr2.086 | NT | NT | NT | NT | NT | NT |
| CLM30 | hr2.090 | NT | NT | NT | NT | NT | NT |
| CLM30 | hr2.091 | NT | NT | NT | NT | NT | NT |
| CLM30 | hr2.094 | NT | NT | NT | NT | NT | NT |
| | S2P6 | 29351 | 49335 | 68964 | 59887 | 39342 | 23177 |
| | CV3-25 | 1333.0 | 806.0 | 1675.0 | 2469.0 | 571.9 | 375.2 |

c

| | | | | SARS-CoV-2 | SARS-CoV-2 | | |
|---|---|---|---|---|---|---|---|
| | | SARS-CoV-2 | SARS-CoV-2 | S Omicron | S Omicron | | HCoV- |
| Participant | Antibody | S Ancestral | S Delta | BA.1 | BA.4/BA.5 | MERS S | 229E S |
| ID | ID | EC₅₀ ng/mL | EC₅₀ ng/mL | EC₅₀ ng/mL | EC₅₀ ng/mL | EC₅₀ ng/mL | EC₅₀ ng/mL |
| CLM25 | fp.001 | 83.68 | 186.2 | 1807 | 407.2 | 43.28 | 27.99 |
| CLM25 | fp.002 | 83.4 | 366.8 | 338.1 | 350 | 50.84 | 40.00 |
| CLM25 | fp.003 | 130.5 | 265.2 | 2079 | 605 | 66.49 | 44.34 |
| CLM25 | fp.004 | 132.5 | 341 | 1744 | 476.2 | 64.95 | 36.98 |
| CLM25 | fp.005 | 88.0 | 212.3 | 276.2 | 421.1 | 51.26 | 41.63 |
| CLM25 | fp.006 | 75.5 | 235 | 486.3 | 345.6 | 41.20 | 34.89 |
| CLM25 | fp.007 | 58.35 | 74.02 | 84.7 | 102.8 | ND | ND |
| CLM25 | fp.008 | 97.0 | 377.9 | 351.2 | 557.9 | 59.24 | 48.19 |
| CLM25 | fp.009 | 127.00 | 221.8 | 194 | 197.7 | 51.48 | 28.48 |
| CLM66 | fp.010 | ND | 5035 | ND | ND | 1963.00 | ND |
| CLM66 | fp.011 | 1004.00 | 4803 | ND | ND | 132.60 | 150.60 |
| CLM30 | hr2.012 | 44.66 | 70.6 | 57.22 | 54.18 | 302.80 | NT |
| CLM30 | hr2.013 | 50.78 | 95.3 | 66.6 | 37.05 | 167.70 | NT |
| CLM30 | hr2.014 | 59.12 | 72.2 | 54.08 | 73.67 | 116.80 | NT |
| CLM30 | hr2.015 | 69.33 | 309.9 | 175.4 | 745.8 | 685.60 | NT |
| CLM30 | hr2.016 | 29.32 | 60.2 | 39.59 | 104.7 | 69.40 | ND |
| CLM30 | hr2.017 | 36.32 | 94.4 | 90.76 | 79.3 | ND | ND |
| CLM41 | hr2.018 | 54.13 | 158.5 | 116.6 | 177.2 | ND | NT |
| CLM41 | hr2.019 | 38.44 | 103.60 | 84.47 | 77.28 | 506.00 | NT |
| CLM41 | hr2.020 | 50.13 | 123.10 | 113.3 | 102.9 | ND | NT |
| CLM33 | hr2.021 | 43.05 | 115.6 | 91.24 | 92.87 | 58.37 | ND |
| CLM33 | hr2.023 | 21.10 | 46.42 | 36.01 | 39.28 | 51.62 | ND |
| CLM33 | hr2.024 | 39.59 | 79.98 | 54.37 | 90.03 | 80.22 | ND |
| CLM65 | hr2.025 | 175.20 | 537.9 | 786.4 | 865.1 | ND | NT |
| CLM65 | hr2.027 | 134.80 | 370.1 | 474.8 | 540.9 | ND | NT |
| CLM30 | hr2.086 | 28.76 | 46.81 | 34.17 | 34.34 | 1159.00 | ND |
| CLM30 | hr2.090 | 38.20 | 44.53 | 57.08 | 71.28 | ND | ND |
| CLM30 | hr2.091 | 88.67 | 58.49 | 68.93 | 126 | 524.00 | ND |
| CLM30 | hr2.094 | 69.64 | 73.25 | 99.63 | 143.5 | 124.00 | ND |
| | S2P6 | 37.86 | 48.18 | 31.9 | 63.43 | 65.98 | ND |
| | CV3-25 | 13.90 | 63.71 | 52.61 | 213.2 | ND | ND |

d

| | | SARS-CoV-2 | SARS-CoV | MERS | HKU1 | OC43 | 229E |
|---|---|---|---|---|---|---|---|
| Participant | Antibody | peptide | peptide | peptide | peptide | peptide | peptide |
| ID | ID | EC₅₀ ng/mL | EC₅₀ | EC₅₀ ng/mL | EC₅₀ ng/mL | EC₅₀ ng/mL | EC₅₀ ng/mL |
| CLM25 | fp.001 | 24.93 | 28.85 | 36.92 | 29.76 | 17.49 | 23.15 |
| CLM25 | fp.002 | 31.96 | 28.52 | 35.83 | 25.56 | 21.14 | 17.64 |
| CLM25 | fp.003 | 36.9 | 28.53 | 34.9 | 23.8 | 14.85 | 20.07 |
| CLM25 | fp.004 | 35.16 | 29.98 | 38.59 | 27.25 | 22.16 | 20.23 |
| CLM25 | fp.005 | 29.51 | 27.55 | 40 | 27.63 | 24.36 | 17.55 |
| CLM25 | fp.006 | 33.1 | 15.68 | 22.81 | 16.75 | 13.42 | 12.15 |
| CLM25 | fp.007 | 176.9 | 77.22 | ND | ND | ND | ND |
| CLM25 | fp.008 | 36.58 | 26.66 | 36.08 | 21.86 | 23.53 | 18.96 |
| CLM25 | fp.009 | 37.98 | 25.08 | 34.62 | 24.14 | 25.98 | 23.59 |
| CLM66 | fp.010 | 119.1 | 38.83 | 59.31 | 37.23 | 42.06 | 135.1 |
| CLM66 | fp.011 | 36.3 | 23.04 | 36.26 | 19.67 | 21.8 | 50.31 |

| | | | | | | |
|---|---|---|---|---|---|---|
| CLM30 | hr2.012 | 19.56 | 27.44 | 30.84 | 34.81 | ND |
| CLM30 | hr2.013 | 31.69 | 37.21 | 35.69 | 52.66 | ND |
| CLM30 | hr2.014 | 35.67 | 58.68 | ND | ND | ND |
| CLM30 | hr2.015 | 47.00 | 62.04 | 34.13 | 71.24 | ND |
| CLM30 | hr2.016 | 6.59 | 6.31 | ND | 9.00 | ND |
| CLM30 | hr2.017 | 14.50 | 455.90 | ND | 20.81 | ND |
| CLM41 | hr2.018 | 33.55 | 42.72 | ND | ND | ND |
| CLM41 | hr2.019 | 26.65 | 27.69 | ND | ND | ND |
| CLM41 | hr2.020 | 35.27 | 98.25 | ND | 40.95 | ND |
| CLM33 | hr2.021 | 24.21 | 24.54 | 23.06 | 26.69 | ND |
| CLM33 | hr2.023 | 17.46 | 22.92 | 7.70 | 16.47 | ND |
| CLM33 | hr2.024 | 106.30 | 56.12 | 45.47 | 31.03 | ND |
| CLM65 | hr2.025 | 117.20 | ND | ND | 51.79 | ND |
| CLM65 | hr2.027 | 61.62 | ND | ND | 28.17 | ND |
| CLM30 | hr2.086 | 23.7 | 6.1 | ND | 877.4 | ND |
| CLM30 | hr2.090 | 85.0 | ND | ND | 311.8 | ND |
| CLM30 | hr2.091 | 84.0 | 23.9 | ND | 294.2 | ND |
| CLM30 | hr2.094 | 83.5 | 24.0 | ND | 20.0 | ND |
| | S2P6 | 16.63 | 20.16 | 9.84 | 17.52 | ND |
| | CV3-25 | 9.68 | 42.29 | ND | 8.95 | ND |

e

| | | | | | | | CRCoV- |
|---|---|---|---|---|---|---|---|
| | | NL63 | CCoV_HuPn | Hu_PDCoV | SADS | FCoV-KU2 | 240/05 |
| Participant | Antibody | peptide | peptide | peptide | peptide | peptide | peptide |
| ID | ID | EC₅₀ ng/mL | EC₅₀ ng/mL | EC₅₀ | EC₅₀ ng/mL | EC₅₀ ng/mL | EC₅₀ ng/mL |
| CLM25 | fp.001 | 21.44 | 21.28 | 31.99 | 25.54 | 31.84 | 31.94 |
| CLM25 | fp.002 | 24.47 | 27.14 | 21.55 | 25.18 | 20.48 | 15.8 |
| CLM25 | fp.003 | 24.93 | 22.68 | 29.78 | 30.53 | 29.51 | 26.57 |
| CLM25 | fp.004 | 26.7 | 22.86 | 34.34 | 27.56 | 28.79 | 26.21 |
| CLM25 | fp.005 | 23.09 | 23.82 | 28.12 | 25.23 | 28.05 | 25.94 |
| CLM25 | fp.006 | 14.37 | 13.97 | 14.42 | 15.4 | 14.95 | 14.85 |
| CLM25 | fp.007 | ND | ND | ND | ND | ND | ND |
| CLM25 | fp.008 | 22.65 | 24.11 | 25.42 | 24.05 | 26.73 | 25.96 |
| CLM25 | fp.009 | 21.42 | 20.97 | 27.56 | 24.69 | 28.49 | 26 |
| CLM66 | fp.010 | 45.08 | ND | 63.13 | 64.45 | 124.9 | 40.65 |
| CLM66 | fp.011 | 26.1 | 145.3 | 27.02 | 27.56 | 32.81 | 21.44 |
| CLM30 | hr2.012 | ND | ND | ND | 22.96 | ND | 27.27 |
| CLM30 | hr2.013 | ND | ND | ND | 41.95 | ND | 57.28 |
| CLM30 | hr2.014 | ND | ND | ND | 704.7 | ND | ND |
| CLM30 | hr2.015 | ND | ND | ND | 73.11 | ND | 57.46 |
| CLM30 | hr2.016 | ND | ND | ND | 73.85 | ND | 5.125 |
| CLM30 | hr2.017 | ND | ND | ND | ND | ND | 18.02 |
| CLM41 | hr2.018 | ND | ND | ND | ND | ND | ND |
| CLM41 | hr2.019 | ND | ND | ND | ND | ND | ND |
| CLM41 | hr2.020 | ND | ND | ND | ND | ND | 40.92 |
| CLM33 | hr2.021 | ND | ND | ND | ND | ND | 24.48 |
| CLM33 | hr2.023 | ND | ND | ND | ND | ND | 16.46 |
| CLM33 | hr2.024 | ND | ND | ND | ND | ND | 54.9 |
| CLM65 | hr2.025 | ND | ND | ND | ND | ND | 74.84 |
| CLM65 | hr2.027 | ND | ND | ND | ND | ND | 41.32 |
| CLM30 | hr2.086 | ND | ND | ND | ND | ND | 245.6 |
| CLM30 | hr2.090 | ND | ND | ND | ND | ND | 658.3 |
| CLM30 | hr2.091 | ND | ND | ND | ND | ND | 87.91 |
| CLM30 | hr2.094 | ND | ND | ND | ND | ND | 14.07 |
| | S2P6 | ND | ND | ND | ND | ND | 18.88 |
| | CV3-25 | ND | ND | ND | ND | ND | 1915 |

f

| | | gamma-IBV | HKU24 | MHV | JL14 | Zhejiang13 | Erinaceus |
|---|---|---|---|---|---|---|---|
| Participant | Antibody | peptide | peptide | peptide | peptide | peptide | peptide |
| ID | ID | EC₅₀ ng/mL | EC₅₀ ng/mL | EC₅₀ ng/mL | EC₅₀ ng/mL | EC₅₀ ng/mL | EC₅₀ ng/mL |
| CLM25 | fp.001 | 18.82 | NT | NT | NT | NT | NT |
| CLM25 | fp.002 | 18.06 | NT | NT | NT | NT | NT |
| CLM25 | fp.003 | 15.85 | NT | NT | NT | NT | NT |
| CLM25 | fp.004 | 20.72 | NT | NT | NT | NT | NT |
| CLM25 | fp.005 | 19.40 | NT | NT | NT | NT | NT |
| CLM25 | fp.006 | 20.19 | NT | NT | NT | NT | NT |
| CLM25 | fp.007 | ND | NT | NT | NT | NT | NT |
| CLM25 | fp.008 | 16.98 | NT | NT | NT | NT | NT |
| CLM25 | fp.009 | 18.08 | NT | NT | NT | NT | NT |
| CLM66 | fp.010 | 186.40 | NT | NT | NT | NT | NT |
| CLM66 | fp.011 | 23.67 | NT | NT | NT | NT | NT |
| CLM30 | hr2.012 | ND | 24.94 | 17.75 | 11.93 | ND | 40.2 |
| CLM30 | hr2.013 | 72.63 | 26.99 | 42.98 | 20.78 | ND | 51.49 |
| CLM30 | hr2.014 | ND | ND | ND | ND | ND | 116.8 |
| CLM30 | hr2.015 | ND | 48.2 | 54.32 | 21.21 | ND | 83.08 |
| CLM30 | hr2.016 | 8.054 | 9.582 | 26.6 | 7.274 | ND | 166.8 |
| CLM30 | hr2.017 | 20.61 | 17.65 | 19.98 | 12.57 | ND | ND |
| CLM41 | hr2.018 | ND | ND | ND | ND | ND | ND |
| CLM41 | hr2.019 | ND | ND | ND | ND | ND | ND |
| CLM41 | hr2.020 | ND | 32.87 | 28.52 | 35.09 | ND | ND |
| CLM33 | hr2.021 | 19.87 | 19.51 | 33.25 | 27.34 | ND | 37.28 |
| CLM33 | hr2.023 | 22.84 | 12.4 | 10.65 | 21.36 | ND | 22.69 |
| CLM33 | hr2.024 | 32.76 | 21.76 | 25.9 | 38.4 | ND | 32.24 |
| CLM65 | hr2.025 | ND | 71.89 | 69.09 | 52.2 | ND | ND |
| CLM65 | hr2.027 | ND | 91.94 | 88.19 | 61.05 | ND | ND |
| CLM30 | hr2.086 | 45.49 | ND | ND | 238.7 | ND | ND |
| CLM30 | hr2.090 | ND | ND | 761.2 | 942.4 | ND | ND |
| CLM30 | hr2.091 | ND | 60.31 | 948 | 96.59 | ND | ND |
| CLM30 | hr2.094 | 11.52 | 11.99 | 18.94 | 15.89 | ND | 19.80 |
| | S2P6 | 6.284 | 10.07 | 21.45 | 10.8 | ND | 34.41 |
| | CV3-25 | ND | 52.09 | 23.94 | 17.64 | ND | 97.76 |

g

| | | HKU4 | HKU5 | Eidolon | Rosettus | HKU9 |
|---|---|---|---|---|---|---|
| Participant | Antibody | peptide | peptide | peptide | peptide | peptide |
| ID | ID | EC₅₀ ng/mL | EC₅₀ ng/mL | EC₅₀ ng/mL | EC₅₀ ng/mL | EC₅₀ ng/mL |
| CLM25 | fp.001 | NT | NT | NT | NT | NT |
| CLM25 | fp.002 | NT | NT | NT | NT | NT |
| CLM25 | fp.003 | NT | NT | NT | NT | NT |
| CLM25 | fp.004 | NT | NT | NT | NT | NT |
| CLM25 | fp.005 | NT | NT | NT | NT | NT |
| CLM25 | fp.006 | NT | NT | NT | NT | NT |
| CLM25 | fp.007 | NT | NT | NT | NT | NT |
| CLM25 | fp.008 | NT | NT | NT | NT | NT |
| CLM25 | fp.009 | NT | NT | NT | NT | NT |
| CLM66 | fp.010 | NT | NT | NT | NT | NT |
| CLM66 | fp.011 | NT | NT | NT | NT | NT |
| CLM30 | hr2.012 | 18.43 | 20.44 | 463.4 | ND | ND |
| CLM30 | hr2.013 | 18.07 | 29.23 | 186.2 | ND | ND |
| CLM30 | hr2.014 | 45.73 | 31.23 | ND | ND | ND |
| CLM30 | hr2.015 | 50.04 | 46.7 | ND | ND | ND |
| CLM30 | hr2.016 | 7.441 | 25.63 | 32.98 | ND | ND |
| CLM30 | hr2.017 | 20.31 | 24.91 | ND | ND | ND |
| CLM41 | hr2.018 | 41.7 | ND | ND | ND | ND |
| CLM41 | hr2.019 | 15.38 | 50.47 | ND | ND | ND |
| CLM41 | hr2.020 | 35.17 | 63.14 | ND | ND | ND |
| CLM33 | hr2.021 | 20.99 | 55.09 | ND | ND | ND |
| CLM33 | hr2.023 | 14.9 | 42.2 | 126.4 | ND | ND |
| CLM33 | hr2.024 | 18.4 | 48.03 | ND | ND | ND |
| CLM65 | hr2.025 | ND | ND | 2924 | ND | ND |
| CLM65 | hr2.027 | ND | ND | 1210 | ND | ND |
| CLM30 | hr2.086 | 26.92 | ND | ND | ND | ND |
| CLM30 | hr2.090 | 424.6 | ND | ND | ND | ND |
| CLM30 | hr2.091 | ND | ND | ND | ND | ND |
| CLM30 | hr2.094 | 15.65 | 19.19 | ND | 656.30 | 227.10 |
| | S2P6 | 9.751 | 34.54 | ND | ND | ND |
| | CV3-25 | ND | 1569 | 35.09 | ND | 358.3 |

(NT = not tested; ND = not determined)

TABLE 2
Neutralizing and binding data of the monoclonal antibodies
a

| | | Ancestral | Alpha | Beta | Gamma | Delta | Omicron BA.1 |
|---|---|---|---|---|---|---|---|
| Participant | Antibody | Pseudovirus | Pseudovirus | Pseudovirus | Pseudovirus | Pseudovirus | Pseudovirus |
| ID | ID | IC₅₀ ng/mL | IC₅₀ ng/mL | IC₅₀ ng/mL | IC₅₀ ng/mL | IC₅₀ ng/mL | IC₅₀ ng/mL |
| CLM30 | sd1.032 | ND | NT | NT | NT | NT | NT |
| CLM30 | sd1.033 | ND | NT | NT | NT | NT | NT |
| CLM30 | sd1.034 | 386.9 | NT | NT | NT | NT | NT |
| CLM30 | sd1.039 | ND | NT | NT | NT | NT | NT |
| CLM30 | sd1.040 | 245.1 | 5844 | 2355 | 66.4 | 3721 | 1004 |
| CLM37 | sd1.047 | ND | NT | NT | NT | NT | NT |
| CLM30 | sd1.100 | NT | NT | NT | NT | NT | NT |
| CLM30 | sd1.102 | NT | NT | NT | NT | NT | NT |
| CLM30 | sd1.103 | NT | NT | NT | NT | NT | NT |
| CLM30 | sd1.107 | NT | NT | NT | NT | NT | NT |
| CLM30 | sd1.108 | 445.8 | NT | NT | NT | NT | NT |
| CLM30 | sd1.127 | 6682 | NT | NT | NT | NT | NT |
| CLM30 | sd1.129 | NT | NT | NT | NT | NT | NT |
| CLM30 | sd1.132 | NT | NT | NT | NT | NT | NT |
| CLM30 | sd1.150 | NT | NT | NT | NT | NT | NT |
| CLM30 | sd1.151 | ND | NT | NT | NT | NT | NT |
| CLM30 | rbd.031 | 861.9 | NT | NT | NT | NT | NT |
| CLM30 | rbd.035 | 1.518 | NT | NT | NT | NT | NT |
| CLM30 | rbd.038 | 0.5518 | NT | NT | NT | NT | NT |
| CLM30 | rbd.042 | 281 | 34.51 | 94.71 | 4.031 | 49519 | 252.6 |
| CLM30 | rbd.043 | 0.5734 | NT | NT | NT | NT | NT |
| CLM30 | rbd.045 | 5.574 | NT | NT | NT | NT | NT |
| CLM37 | rbd.046 | ND | NT | NT | NT | NT | NT |
| CLM37 | rbd.048 | 2.75 | NT | NT | NT | NT | NT |
| CLM37 | rbd.049 | 3711 | NT | NT | NT | NT | NT |
| CLM30 | rbd.104 | 6202 | NT | NT | NT | NT | NT |
| CLM30 | rbd.106 | 67.44 | NT | NT | NT | NT | NT |
| CLM30 | rbd.110 | 4254 | NT | NT | NT | NT | NT |
| CLM30 | rbd.112 | 86.73 | NT | NT | NT | NT | NT |
| CLM30 | rbd.113 | 373.1 | NT | NT | NT | NT | NT |
| CLM30 | rbd.117 | 202.8 | NT | NT | NT | NT | NT |
| CLM30 | rbd.123 | 1210 | NT | NT | NT | NT | NT |
| CLM30 | rbd.141 | 163.6 | NT | NT | NT | NT | NT |
| CLM30 | rbd.142 | 5093 | NT | NT | NT | NT | NT |
| CLM30 | rbd.143 | 279.7 | NT | NT | NT | NT | NT |
| CoV-X404 [symbol] | | 50.94 | 26.27 | 76.57 | 15.81 | 2479 | 197.3 |
| sd1.040 + rbd. [symbol] | | 99.1 | 30.03 | 105.4 | 7.336 | 4452 | 110.9 |
| | C121 | NT | NT | NT | NT | NT | NT |
| | C135 | 0.6594 | NT | NT | NT | NT | NT |

b

| | | Omicron | Omicron | Omicron | Omicron | SARS-CoV-2 | SARS-CoV-2 |
|---|---|---|---|---|---|---|---|
| | | BA.2 | BA.2.75 | BA.2.75.2 | BA.4/BA.5 | RBD | SD1-RBD |
| Participant | Antibody | Pseudovirus | Pseudovirus | Pseudovirus | Pseudovirus | Ancestral | Ancestral |
| ID | ID | IC₅₀ ng/mL | IC50 ng/mL | IC50 ng/mL | IC₅₀ ng/mL | EC₅₀ ng/mL | EC₅₀ ng/mL |
| CLM30 | sd1.032 | NT | NT | NT | NT | ND | 629.7 |
| CLM30 | sd1.033 | NT | NT | NT | NT | ND | 36.23 |
| CLM30 | sd1.034 | NT | NT | NT | NT | ND | 19.25 |
| CLM30 | sd1.039 | NT | NT | | NT | ND | 20.76 |
| CLM30 | sd1.040 | 1436 | 4018 | 4486 | 1357 | ND | 23.59 |
| CLM37 | sd1.047 | NT | NT | NT | NT | ND | 270.2 |
| CLM30 | sd1.100 | NT | NT | NT | NT | ND | 227.6 |
| CLM30 | sd1.102 | NT | NT | NT | NT | ND | 131.9 |
| CLM30 | sd1.103 | NT | NT | NT | NT | ND | 73.17 |
| CLM30 | sd1.107 | NT | NT | NT | NT | ND | 767.3 |
| CLM30 | sd1.108 | NT | NT | NT | NT | ND | 25.3 |
| CLM30 | sd1.127 | NT | NT | NT | NT | ND | 20.5 |
| CLM30 | sd1.129 | NT | NT | NT | NT | ND | 64.88 |
| CLM30 | sd1.132 | NT | NT | NT | NT | ND | 120.7 |
| CLM30 | sd1.150 | NT | NT | NT | NT | ND | 63.28 |
| CLM30 | sd1.151 | NT | NT | NT | NT | ND | 33.09 |
| CLM30 | rbd.031 | NT | NT | NT | NT | 7.969 | 8.377 |
| CLM30 | rbd.035 | NT | NT | NT | NT | 17 | 23.11 |
| CLM30 | rbd.038 | NT | NT | NT | NT | 37.78 | 29.24 |
| CLM30 | rbd.042 | 556.5 | 1043 | 2438 | ND | 27.34 | 19.18 |
| CLM30 | rbd.043 | NT | NT | NT | NT | 23.04 | 16.98 |
| CLM30 | rbd.045 | NT | NT | NT | NT | 33.03 | 25.85 |
| CLM37 | rbd.046 | NT | NT | NT | NT | 64.58 | 22.31 |
| CLM37 | rbd.048 | NT | NT | NT | NT | 15.08 | 17.43 |
| CLM37 | rbd.049 | NT | NT | NT | NT | 89.82 | 71.15 |
| CLM30 | rbd.104 | NT | NT | NT | NT | 45.98 | 33.64 |
| CLM30 | rbd.106 | NT | NT | NT | NT | 50.58 | 51.85 |
| CLM30 | rbd.110 | NT | NT | NT | NT | 40.75 | 33.91 |
| CLM30 | rbd.112 | NT | NT | NT | NT | 37.91 | 32.45 |
| CLM30 | rbd.113 | NT | NT | NT | NT | 42.47 | 31.66 |
| CLM30 | rbd.117 | NT | NT | NT | NT | 37.6 | 37.58 |
| CLM30 | rbd.123 | NT | NT | NT | NT | 14.77 | 14.96 |
| CLM30 | rbd.141 | NT | NT | NT | NT | 6.462 | 18.73 |
| CLM30 | rbd.142 | NT | NT | NT | NT | 13.45 | 36.13 |
| CLM30 | rbd.143 | NT | NT | NT | NT | 12.75 | 37.13 |
| CoV-X404 [symbol] | | 539.8 | 277.3 | 785.1 | 3786 | NT | NT |
| sd1.040 + rbd. [symbol] | | 468.1 | 575.2 | 1114 | 1723 | NT | NT |
| | C121 | NT | NT | NT | NT | NT | 21.24 |
| | C135 | NT | NT | NT | NT | NT | 15.37 |

c

| | | SARS-CoV-2 | SARS-CoV-2 | | | | |
|---|---|---|---|---|---|---|---|
| | | SD1-RBD | SD1-RBD | SARS-CoV-2 | SARS-CoV-2 | SARS-CoV-2 | SARS-CoV-2 |
| | | Ancestral | Ancestral | SD1-RBD | SD1-RBD | SD1-RBD | SD1-RBD |
| Participant | Antibody | T572I | E583D | Alpha | Beta | Gamma | Delta |
| ID | ID | EC₅₀ ng/mL | EC₅₀ ng/mL | EC₅₀ ng/mL | EC₅₀ ng/mL | EC₅₀ ng/mL | EC₅₀ ng/mL |
| CLM30 | sd1.032 | 1644 | ND | 780 | 344.6 | 397.9 | 444.5 |
| CLM30 | sd1.033 | 72.98 | 1078 | 65.93 | 38.48 | 34.47 | 22.21 |
| CLM30 | sd1.034 | 19.35 | 34.76 | 20.28 | 19.06 | 20.64 | 11.15 |
| CLM30 | sd1.039 | 19.88 | 27.86 | 23.32 | 23.55 | 23.54 | 18.63 |
| CLM30 | sd1.040 | 23.72 | 27.93 | 31.4 | 20.31 | 19.11 | 15.75 |
| CLM37 | sd1.047 | 54.93 | ND | 119.4 | 454.3 | 425.8 | 310.2 |
| CLM30 | sd1.100 | 115.4 | 441.8 | 166.9 | ND | ND | 2404 |
| CLM30 | sd1.102 | 91.89 | 891.3 | 92.93 | 3165 | 1258 | 810.8 |
| CLM30 | sd1.103 | 57.11 | 345.2 | 78.42 | 358.4 | 244 | 300.3 |
| CLM30 | sd1.107 | 657.2 | 2978 | 898 | ND | 5962 | ND |
| CLM30 | sd1.108 | 11.75 | 20.93 | 19.32 | 26.52 | 18.33 | 24.09 |
| CLM30 | sd1.127 | 6.072 | 7.751 | 12.06 | 10.33 | 9.352 | 12.42 |
| CLM30 | sd1.129 | 25.53 | 190.1 | 53.2 | 1842 | 323.9 | 280.2 |
| CLM30 | sd1.132 | 77.7 | 1146 | 171.2 | 1825 | 932.3 | 919.1 |
| CLM30 | sd1.150 | 34.02 | 122.7 | 39.07 | 163 | 84.37 | 52.18 |
| CLM30 | sd1.151 | 20.01 | 24.49 | 29.24 | 23.53 | 19.84 | 32.26 |
| CLM30 | rbd.031 | NT | NT | 25.14 | 20.92 | 17.44 | 14.95 |
| CLM30 | rbd.035 | NT | NT | 29.5 | 12.98 | 14.2 | 16.5 |
| CLM30 | rbd.038 | NT | NT | 31.81 | 35.71 | 17.31 | 15.97 |
| CLM30 | rbd.042 | NT | NT | 14.2 | 13.64 | 19.66 | 17.77 |
| CLM30 | rbd.043 | NT | NT | 19.9 | ND | 971.8 | ND |
| CLM30 | rbd.045 | NT | NT | 36.19 | 32.29 | 25.79 | 23.23 |
| CLM37 | rbd.046 | NT | NT | 27.64 | 39.78 | 23.01 | 23.52 |
| CLM37 | rbd.048 | NT | NT | 14.49 | 936.3 | 78.45 | 12.24 |
| CLM37 | rbd.049 | NT | NT | 16.49 | 1182 | 1489 | 16.96 |
| CLM30 | rbd.104 | NT | NT | 22.34 | 28.55 | 20.7 | 26.86 |
| CLM30 | rbd.106 | NT | NT | 23.34 | 25.82 | 21.3 | 30.96 |
| CLM30 | rbd.110 | NT | NT | 24.09 | 22.29 | 17.52 | 26.92 |
| CLM30 | rbd.112 | NT | NT | 24.48 | 25.79 | 22.09 | 36.42 |
| CLM30 | rbd.113 | NT | NT | 24.19 | 21.75 | 18.31 | 27.86 |
| CLM30 | rbd.117 | NT | NT | 21.89 | 25.62 | 25.2 | 40.72 |
| CLM30 | rbd.123 | NT | NT | 10.91 | 9.4 | 8.52 | 10.64 |
| CLM30 | rbd.141 | NT | NT | 15.86 | 17.07 | 11.62 | 16.34 |
| CLM30 | rbd.142 | NT | NT | 27.07 | 36.76 | 21.06 | 29.95 |
| CLM30 | rbd.143 | NT | NT | 24.98 | 22.84 | 19.59 | 29.11 |
| CoV-X404 [symbol] | | NT | NT | NT | NT | NT | NT |
| sd1.040 + rbd. [symbol] | | NT | NT | NT | NT | NT | NT |
| | C121 | NT | NT | 18.65 | ND | ND | 18.13 |
| | C135 | NT | NT | 15.17 | 14.84 | 13.57 | 13.65 |

d

| | | SARS-CoV-2 | SARS-CoV-2 | SARS-CoV-2 | SARS-CoV-2 | SARS-CoV-2 | SARS-CoV-2 |
|---|---|---|---|---|---|---|---|
| | | SD1-RBD | SD1-RBD | SD1-RBD | SD1-RBD | SD1-RBD | SD1-RBD |
| | | Omicron | Omicron | Omicron | Omicron | Omicron | Omicron |
| Participant | Antibody | BA.1 | BA.2 | BA.2.75 | BA.2.75.2 | BA.4/BA.5 | BA.4.6 |
| ID | ID | EC₅₀ ng/mL | EC₅₀ ng/mL | EC₅₀ ng/mL | EC₅₀ ng/mL | EC50 ng/mL | EC₅₀ ng/mL |
| CLM30 | sd1.032 | ND | ND | ND | ND | ND | ND |
| CLM30 | sd1.033 | 654.5 | 382.8 | ND | ND | 319.6 | ND |
| CLM30 | sd1.034 | 22.54 | 19.33 | 35.86 | 34.36 | 35.88 | 34.84 |
| CLM30 | sd1.039 | 24.5 | 22.42 | 46.36 | 27.33 | 43.1 | 37.25 |
| CLM30 | sd1.040 | 20.97 | 15.34 | 48.74 | 27.17 | 38.42 | 38.04 |
| CLM37 | sd1.047 | ND | ND | ND | ND | ND | ND |
| CLM30 | sd1.100 | 149.7 | 73.94 | 187.1 | 190.4 | 134 | 198.7 |
| CLM30 | sd1.102 | ND | ND | 2934 | ND | 4908 | ND |
| CLM30 | sd1.103 | 8284 | 508.2 | 1649 | ND | 2833 | ND |
| CLM30 | sd1.107 | ND | 6302 | ND | ND | ND | ND |
| CLM30 | sd1.108 | 25.77 | 20.12 | 45.89 | 28.24 | 17.26 | 30.81 |
| CLM30 | sd1.127 | 11.18 | 9.606 | 19.49 | 10.43 | 7.411 | 13.1 |
| CLM30 | sd1.129 | 2004 | 1304 | 5097 | ND | 2795 | ND |
| CLM30 | sd1.132 | 11491 | 3127 | ND | ND | 3753 | ND |
| CLM30 | sd1.150 | 1241 | 233.6 | 255.7 | 1073 | 732.3 | 675.8 |
| CLM30 | sd1.151 | 25.79 | 26.31 | 66.25 | 16.79 | 18.32 | 24.2 |
| CLM30 | rbd.031 | 25.21 | 14.25 | 48.62 | 52.21 | 30.89 | 32.99 |
| CLM30 | rbd.035 | ND | ND | ND | ND | 46.11 | ND |
| CLM30 | rbd.038 | ND | ND | 123.8 | ND | 153.6 | ND |
| CLM30 | rbd.042 | 28.82 | 22.95 | 66.31 | 56.04 | ND | ND |
| CLM30 | rbd.043 | ND | 303.2 | ND | ND | ND | ND |
| CLM30 | rbd.045 | ND | 38.41 | 220.2 | ND | 65.03 | 383.1 |
| CLM37 | rbd.046 | 314.1 | 62.51 | 66.06 | 377.4 | 87.43 | 116 |
| CLM37 | rbd.048 | ND | ND | ND | ND | ND | ND |
| CLM37 | rbd.049 | ND | ND | ND | ND | ND | ND |
| CLM30 | rbd.104 | 26.92 | 22.17 | 39.06 | NT | 31.45 | NT |
| CLM30 | rbd.106 | 28.17 | 23.64 | 54.11 | NT | 30.78 | NT |
| CLM30 | rbd.110 | 18.89 | 19.27 | 52.48 | NT | 14.97 | NT |
| CLM30 | rbd.112 | 28.89 | 30.15 | 64.73 | NT | 21.29 | NT |
| CLM30 | rbd.113 | 19.94 | 19.84 | 51.09 | NT | 16.94 | NT |
| CLM30 | rbd.117 | 25.81 | 27.15 | 52.22 | NT | 23.43 | NT |
| CLM30 | rbd.123 | 9.418 | 8.765 | 13.64 | NT | 8.93 | NT |
| CLM30 | rbd.141 | 18.75 | 16.85 | 15.3 | NT | 19.67 | NT |
| CLM30 | rbd.142 | 23.2 | 22.76 | 29.08 | NT | 21.54 | NT |
| CLM30 | rbd.143 | 22.33 | 24.89 | 37.52 | NT | 26.08 | NT |
| CoV-x404 [symbol] | | NT | NT | NT | NT | NT | NT |
| sd1.040 + rbd. [symbol] | | NT | NT | NT | NT | NT | NT |
| | C121 | ND | ND | ND | ND | ND | ND |
| | C135 | ND | ND | ND | ND | ND | ND |

e

| | | SARS-CoV-2 | SARS-CoV-2 | | | SARS- | SARS- |
|---|---|---|---|---|---|---|---|
| | | SD1-RBD | SD1-RBD | SARS- | SARS- | CoV-2 S | CoV-2 S |
| | | Omicron | Omicron | CoV-2 S | CoV-2 S | Omicron | Omicron |
| Participant | Antibody | BA.4.7 | BA.5.9 | Ancestral | Delta | BA.1 | BA.4/BA.5 |
| ID | ID | EC₅₀ ng/mL | EC₅₀ ng/mL | EC₅₀ ng/mL | EC₅₀ ng/mL | EC₅₀ ng/mL | EC₅₀ ng/mL |
| CLM30 | sd1.032 | ND | ND | 1233 | 4471 | 8980 | ND |
| CLM30 | sd1.033 | ND | ND | 72.26 | 85.88 | 1265 | 438.2 |
| CLM30 | sd1.034 | 33.45 | 29.8 | 22.52 | 23.24 | 68.31 | 41.59 |
| CLM30 | sd1.039 | 39.07 | 30.46 | 39.35 | 42.26 | 59.73 | 32.3 |
| CLM30 | sd1.040 | 35.71 | 27.31 | 17.45 | 17.82 | 31.58 | 23.4 |
| CLM37 | sd1.047 | ND | ND | 165.3 | 438.7 | ND | 6125 |
| CLM30 | sd1.100 | 186.1 | 209.9 | 340.6 | 309 | 1842 | 309.2 |
| CLM30 | sd1.102 | ND | ND | 68.35 | 99.6 | 2219 | 537 |
| CLM30 | sd1.103 | ND | ND | 50.01 | 62.37 | 2029 | 239 |
| CLM30 | sd1.107 | ND | ND | 491.3 | 1559 | 2796 | 2964 |
| CLM30 | sd1.108 | 36.49 | 28.69 | 16.04 | 15.48 | 38.22 | 21.69 |
| CLM30 | sd1.127 | 12.86 | 10.23 | 14.17 | 12.75 | 15.98 | 10.71 |
| CLM30 | sd1.129 | ND | ND | 25.8 | 25.48 | 277.3 | 98.25 |
| CLM30 | sd1.132 | ND | ND | 74.03 | 69.39 | 1010 | 354.2 |
| CLM30 | sd1.150 | 1300 | 944.7 | 27.09 | 25.47 | 55.03 | 68.5 |
| CLM30 | sd1.151 | 22.7 | 18.83 | 24.12 | 20.19 | 37.25 | 19.89 |
| CLM30 | rbd.031 | 31.64 | 27.6 | 19.37 | 20.79 | 2013 | 57.99 |
| CLM30 | rbd.035 | ND | ND | 14.66 | 13.53 | 2718 | ND |
| CLM30 | rbd.038 | ND | ND | 18.27 | 17.51 | 3109 | 3407 |
| CLM30 | rbd.042 | ND | ND | 17.26 | 345.7 | 254.7 | ND |
| CLM30 | rbd.043 | ND | ND | 10.36 | 2898 | 10174 | 3160 |
| CLM30 | rbd.045 | 407.2 | 350.3 | 30.64 | 31.38 | ND | 159 |
| CLM37 | rbd.046 | 144.1 | 105.2 | 40.04 | 57.99 | 1499 | 576.6 |
| CLM37 | rbd.048 | ND | ND | 14.9 | 11.39 | 210.5 | 44070 |
| CLM37 | rbd.049 | ND | ND | 25.7 | 110.2 | ND | 4198 |
| CLM30 | rbd.104 | NT | NT | NT | NT | NT | NT |
| CLM30 | rbd.106 | NT | NT | NT | NT | NT | NT |
| CLM30 | rbd.110 | NT | NT | NT | NT | NT | NT |
| CLM30 | rbd.112 | NT | NT | NT | NT | NT | NT |
| CLM30 | rbd.113 | NT | NT | NT | NT | NT | NT |
| CLM30 | rbd.117 | NT | NT | NT | NT | NT | NT |
| CLM30 | rbd.123 | NT | NT | NT | NT | NT | NT |
| CLM30 | rbd.141 | NT | NT | NT | NT | NT | NT |
| CLM30 | rbd.142 | NT | NT | NT | NT | NT | NT |
| CLM30 | rbd.143 | NT | NT | NT | NT | NT | NT |
| CoV-X404 [symbol] | | NT | NT | NT | NT | NT | NT |
| sd1.040 + rbd. [symbol] | | NT | NT | NT | NT | NT | NT |
| | C121 | ND | ND | 18.67 | 16.02 | ND | ND |
| | C135 | ND | ND | 12.63 | 11.9 | ND | ND |

f

| | | Fab Ka | Fab Kd | Fab KD | Fab Ka | Fab Kd | Fab KD |
|---|---|---|---|---|---|---|---|
| | | (M⁻¹s⁻¹) on | (M⁻¹s⁻¹) on | (nM) on | (M⁻¹s⁻¹) on | (M⁻¹s⁻¹) on | (nM) on |
| Participant | Antibody | ancestral | ancestral | ancestral | ancestral | ancestral | ancestral |
| ID | ID | SD1-RBD | SD1-RBD | SD1-RBD | Spike | Spike | Spike |
| CLM30 | sd1.032 | NT | NT | NT | NT | NT | NT |
| CLM30 | sd1.033 | NT | NT | NT | NT | NT | NT |
| CLM30 | sd1.034 | NT | NT | NT | NT | NT | NT |
| CLM30 | sd1.039 | NT | NT | NT | NT | NT | NT |
| CLM30 | sd1.040 | 8.86E+05 | 3.72E−03 | 4.2 | 7.30E+05 | 1.20E−04 | 0.2 |
| CLM37 | sd1.047 | NT | NT | NT | NT | NT | NT |
| CLM30 | sd1.100 | NT | NT | NT | NT | NT | NT |
| CLM30 | sd1.102 | NT | NT | NT | NT | NT | NT |
| CLM30 | sd1.103 | NT | NT | NT | NT | NT | NT |
| CLM30 | sd1.107 | NT | NT | NT | NT | NT | NT |
| CLM30 | sd1.108 | NT | NT | NT | NT | NT | NT |
| CLM30 | sd1.127 | NT | NT | NT | NT | NT | NT |
| CLM30 | sd1.129 | NT | NT | NT | NT | NT | NT |
| CLM30 | sd1.132 | NT | NT | NT | NT | NT | NT |
| CLM30 | sd1.150 | NT | NT | NT | NT | NT | NT |
| CLM30 | sd1.151 | NT | NT | NT | NT | NT | NT |
| CLM30 | rbd.031 | NT | NT | NT | NT | NT | NT |
| CLM30 | rbd.035 | NT | NT | NT | NT | NT | NT |
| CLM30 | rbd.038 | NT | NT | NT | NT | NT | NT |
| CLM30 | rbd.042 | NT | NT | NT | NT | NT | NT |
| CLM30 | rbd.043 | NT | NT | NT | NT | NT | NT |
| CLM30 | rbd.045 | NT | NT | NT | NT | NT | NT |
| CLM37 | rbd.046 | NT | NT | NT | NT | NT | NT |
| CLM37 | rbd.048 | NT | NT | NT | NT | NT | NT |
| CLM37 | rbd.049 | NT | NT | NT | NT | NT | NT |
| CLM30 | rbd.104 | NT | NT | NT | NT | NT | NT |
| CLM30 | rbd.106 | NT | NT | NT | NT | NT | NT |
| CLM30 | rbd.110 | NT | NT | NT | NT | NT | NT |
| CLM30 | rbd.112 | NT | NT | NT | NT | NT | NT |
| CLM30 | rbd.113 | NT | NT | NT | NT | NT | NT |
| CLM30 | rbd.117 | NT | NT | NT | NT | NT | NT |
| CLM30 | rbd.123 | NT | NT | NT | NT | NT | NT |
| CLM30 | rbd.141 | NT | NT | NT | NT | NT | NT |
| CLM30 | rbd.142 | NT | NT | NT | NT | NT | NT |
| CLM30 | rbd.143 | NT | NT | NT | NT | NT | NT |
| CoV-X404 [symbol] | | NT | NT | NT | NT | NT | NT |
| sd1.040 + rbd. [symbol] | | NT | NT | NT | NT | NT | NT |
| | C121 | NT | NT | NT | NT | NT | NT |
| | C135 | NT | NT | NT | NT | NT | NT |

(NT = not tested; ND = not determined) [symbol]
indicates data missing or illegible when filed

TABLE 3
coldspot regions

| Coldspot | Position in | |
|---|---|---|
| name | S (*) | Sequence |
| cs1 (FP) | 814-838 | KRSFIEDLLFNKVTLADAGFIKQYG |
| cs2 (HR2) | 1141-1161 | QPELDSFKEELDKYFKNHTS |
| cs3 | 113-137 | KTQSLLIVNNATNVVIKVCEFQFCN |
| cs4 | 191-209 | EFVFKNIDGYFKIYSKHTP |
| cs5 | 309-338 | EKGIYQTSNFRVQPTESIVRFPNITNLCPF |
| cs6 | 377-404 | FKCYGVSPTKLNDLCFTNVYADSFVIRG |
| cs7 | 418-438 | IADYNYKLPDDFTGCVIAWNS |
| cs8 | 454-476 | RLFRKSNLKPFERDISTEIYQAG |
| cs9 | 523-546 | TVCGPKKSTNLVKNKCVNFNFNGL |
| cs10 | 584-612 | ILDITPCSFGGVSVITPGTNTSNQVAVLY |
| cs11 | 733-763 | KTSVDCTMYICGDSTECSNLLLQYGSFCTQL |
| cs12 | 860-878 | VLPPLLTDEMIAQYTSALL |
| cs13 | 889-928 | GAGAALQIPFAMQMAYRFNGIGVTQNVLYENQKLIANQFN |
| cs14 | 983-1019 | RLDKVEAEVQIDRLITGRLQSLQTYVTQQLIRAAEIR |
| cs15 | 1028-1070 | KMSECVLGQSKRVDFCGKGYHLMSFPQSAPHGVVFLHVTYVPA |

(*) positions are based on Genbank: QHO60594.1

1. An antibody, or antigen-binding fragment thereof, specifically binding to a conserved region of a coronavirus S protein, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.

2. The antibody, or antigen-binding fragment thereof, of claim 1, wherein the conserved region is at least one selected from the group consisting of: SEQ ID NO: 01, SEQ ID NO: 02, SEQ ID NO: 16 and SEQ ID NO: 298.

3. The antibody, or antigen-binding fragment thereof, of claim 2, the antibody, or antigen-binding fragment thereof, comprising:
a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 as defined in SEQ ID NO: 161 and CDR3 as defined in SEQ ID NO: 162;
ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; or
bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451.

4. The antibody, or antigen-binding fragment thereof, of claim 3, the antibody, or antigen-binding fragment thereof, comprising a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51.

5. The antibody, or antigen-binding fragment thereof, of claim 3, the antibody, or antigen-binding fragment thereof, comprising a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46.

6. The antibody, or antigen-binding fragment thereof, of claim 3, the antibody, or antigen-binding fragment thereof, comprising a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96.

7. The antibody, or antigen-binding fragment thereof, of claim 2, wherein the antibody, or antigen-binding fragment thereof, is cross-specific with the S proteins of SARS-COV-2, MERS and HCoV-229E.

8. The antibody, or antigen-binding fragment thereof, of claim 2, wherein the conserved region is SEQ ID NO: 01 and wherein the antibody, or antigen-binding fragment thereof, is cross-specific for coronaviruses of the alpha, beta, gamma and delta genera.

9. The antibody, or antigen-binding fragment thereof, of claim 2, wherein the conserved region is SEQ ID NO: 02 and wherein the antibody, or antigen-binding fragment thereof, is cross-specific for coronaviruses of the alpha, beta and gamma genera.

10. The antibody, or antigen-binding fragment thereof, of claim 2, wherein a) the conserved region is SEQ ID NO: 16 and/or SEQ ID NO: 298 and b) wherein the antibody, or antigen-binding fragment thereof, is cross-specific for SARS-COV-2 variants.

11. A multispecific antibody or a multispecific antigen-binding fragment thereof, specifically binding to at least two regions of a coronavirus S protein, wherein at least one of the coronavirus S protein is a conserved region, wherein the conserved region is at least one selected from the group consisting of: SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16 and SEQ ID NO: 298.

12. The multispecific antibody or a multispecific antigen-binding fragment thereof, according to claim 11, wherein the at least two regions of the coronavirus S protein comprise an RBD region of a coronavirus.

13. The multispecific antibody or the multispecific antigen-binding fragment thereof, of claim 11, comprising at least one selected from the group of:
a) a variable heavy (VH) chain comprising CDR1 as defined in SEQ ID NO: 17, CDR2 as defined in SEQ ID NO: 18 and CDR3 as defined in SEQ ID NO: 19 and a variable light (VL) chain comprising CDR1 as defined in SEQ ID NO: 20, CDR2 comprising or consisting of the sequence of GAS and CDR3 as defined in SEQ ID NO: 21;
b) a variable heavy (VH) chain comprising CDR1 as defined in SEQ ID NO: 22, CDR2 as defined in SEQ ID NO: 23 and CDR3 as defined in SEQ ID NO: 24 and a variable light (VL) chain comprising CDR1 as defined in SEQ ID NO: 25, CDR2 comprising or consisting of the sequence of GAS and CDR3 as defined in SEQ ID NO: 26;
c) a variable heavy (VH) chain comprising CDR1 as defined in SEQ ID NO: 27, CDR2 as defined in SEQ ID NO: 28 and CDR3 as defined in SEQ ID NO: 29 and a variable light (VL) chain comprising CDR1 as defined in SEQ ID NO: 30, CDR2 comprising or consisting of the sequence of GAS and CDR3 as defined in SEQ ID NO: 31;
d) a variable heavy (VH) chain comprising CDR1 as defined in SEQ ID NO: 32, CDR2 as defined in SEQ ID NO: 33 and CDR3 as defined in SEQ ID NO: 34 and a variable light (VL) chain comprising CDR1 as defined in SEQ ID NO: 35, CDR2 comprising or consisting of the sequence of GAS and CDR3 as defined in SEQ ID NO: 36;
e) a variable heavy (VH) chain comprising CDR1 as defined in SEQ ID NO: 37, CDR2 as defined in SEQ ID NO: 38 and CDR3 as defined in SEQ ID NO: 39 and a variable light (VL) chain comprising CDR1 as defined in SEQ ID NO: 40, CDR2 comprising or consisting of the sequence of GAS and CDR3 as defined in SEQ ID NO: 41;
f) a variable heavy (VH) chain comprising CDR1 as defined in SEQ ID NO: 42, CDR2 as defined in SEQ ID NO: 43 and CDR3 as defined in SEQ ID NO: 44 and a variable light (VL) chain comprising CDR1 as defined in SEQ ID NO: 45, CDR2 comprising or consisting of the sequence of GAS and CDR3 as defined in SEQ ID NO: 46;
g) a variable heavy (VH) chain comprising CDR1 as defined in SEQ ID NO: 47, CDR2 as defined in SEQ ID NO: 48 and CDR3 as defined in SEQ ID NO: 49 and a variable light (VL) chain comprising CDR1 as defined in SEQ ID NO: 50, CDR2 comprising or consisting of the sequence of LGS and CDR3 as defined in SEQ ID NO: 51;
h) a variable heavy (VH) chain comprising CDR1 as defined in SEQ ID NO: 52, CDR2 as defined in SEQ ID NO: 53 and CDR3 as defined in SEQ ID NO: 54 and a variable light (VL) chain comprising CDR1 as defined in SEQ ID NO: 55, CDR2 comprising or consisting of the sequence of GAS and CDR3 as defined in SEQ ID NO: 56;
i) a variable heavy (VH) chain comprising CDR1 as defined in SEQ ID NO: 57, CDR2 as defined in SEQ ID NO: 58 and CDR3 as defined in SEQ ID NO: 59 and a variable light (VL) chain comprising CDR1 as defined in SEQ ID NO: 60, CDR2 comprising or consisting of the sequence of GAS and CDR3 as defined in SEQ ID NO: 61;
j) a variable heavy (VH) chain comprising CDR1 as defined in SEQ ID NO: 62, CDR2 as defined in SEQ ID NO: 63 and CDR3 as defined in SEQ ID NO: 64 and a variable light (VL) chain comprising CDR1 as defined in SEQ ID NO: 65, CDR2 comprising or consisting of the sequence of KAS and CDR3 as defined in SEQ ID NO: 66;
k) a variable heavy (VH) chain comprising CDR1 as defined in SEQ ID NO: 67, CDR2 as defined in SEQ ID NO: 68 and CDR3 as defined in SEQ ID NO: 69 and a variable light (VL) chain comprising CDR1 as defined in SEQ ID NO: 70, CDR2 comprising or consisting of the sequence of DAS and CDR3 as defined in SEQ ID NO: 71;
l) a variable heavy (VH) chain comprising CDR1 as defined in SEQ ID NO: 72, CDR2 as defined in SEQ ID NO: 73 and CDR3 as defined in SEQ ID NO: 74 and a variable light (VL) chain comprising CDR1 as defined in SEQ ID NO: 75, CDR2 comprising or consisting of the sequence of DNI and CDR3 as defined in SEQ ID NO: 76;
m) a variable heavy (VH) chain comprising CDR1 as defined in SEQ ID NO: 77, CDR2 as defined in SEQ ID NO: 78 and CDR3 as defined in SEQ ID NO: 79 and a variable light (VL) chain comprising CDR1 as defined in SEQ ID NO: 80, CDR2 comprising or consisting of the sequence of DNN and CDR3 as defined in SEQ ID NO: 81;
n) a variable heavy (VH) chain comprising CDR1 as defined in SEQ ID NO: 82, CDR2 as defined in SEQ ID NO: 83 and CDR3 as defined in SEQ ID NO: 84 and a variable light (VL) chain comprising CDR1 as defined in SEQ ID NO: 85, CDR2 comprising or consisting of the sequence of WAS and CDR3 as defined in SEQ ID NO: 86;
o) a variable heavy (VH) chain comprising CDR1 as defined in SEQ ID NO: 87, CDR2 as defined in SEQ ID NO: 88 and CDR3 as defined in SEQ ID NO: 89 and a variable light (VL) chain comprising CDR1 as defined in SEQ ID NO: 90, CDR2 comprising or consisting of the sequence of DNS and CDR3 as defined in SEQ ID NO: 91;
p) a variable heavy (VH) chain comprising CDR1 as defined in SEQ ID NO: 92, CDR2 as defined in SEQ ID NO: 93 and CDR3 as defined in SEQ ID NO: 94 and a variable light (VL) chain comprising CDR1 as defined in SEQ ID NO: 95, CDR2 comprising or consisting of the sequence of GAS and CDR3 as defined in SEQ ID NO: 96;
q) a variable heavy (VH) chain comprising CDR1 as defined in SEQ ID NO: 97, CDR2 as defined in SEQ ID NO: 98 and CDR3 as defined in SEQ ID NO: 99 and a variable light (VL) chain comprising CDR1 as defined in SEQ ID NO: 100, CDR2 comprising or consisting of the sequence of WAS and CDR3 as defined in SEQ ID NO: 101;
r) a variable heavy (VH) chain comprising CDR1 as defined in SEQ ID NO: 102, CDR2 as defined in SEQ ID NO: 103 and CDR3 as defined in SEQ ID NO: 104 and a variable light (VL) chain comprising CDR1 as defined in SEQ ID NO: 105, CDR2 comprising or consisting of the sequence of WAS and CDR3 as defined in SEQ ID NO: 106;
s) a variable heavy (VH) chain comprising CDR1 as defined in SEQ ID NO: 107, CDR2 as defined in SEQ ID NO: 108 and CDR3 as defined in SEQ ID NO: 109 and a variable light (VL) chain comprising CDR1 as defined in SEQ ID NO: 110, CDR2 comprising or consisting of the sequence of WAS and CDR3 as defined in SEQ ID NO: 111;
t) a variable heavy (VH) chain comprising CDR1 as defined in SEQ ID NO: 112, CDR2 as defined in SEQ ID NO: 113 and CDR3 as defined in SEQ ID NO: 114 and a variable light (VL) chain comprising CDR1 as defined in SEQ ID NO: 115, CDR2 comprising or consisting of the sequence of RNN and CDR3 as defined in SEQ ID NO: 116;
u) a variable heavy (VH) chain comprising CDR1 as defined in SEQ ID NO: 117, CDR2 as defined in SEQ ID NO: 118 and CDR3 as defined in SEQ ID NO: 119 and a variable light (VL) chain comprising CDR1 as defined in SEQ ID NO: 120, CDR2 comprising or consisting of the sequence of GAS and CDR3 as defined in SEQ ID NO: 121;
v) a variable heavy (VH) chain comprising CDR1 as defined in SEQ ID NO: 122, CDR2 as defined in SEQ ID NO: 123 and CDR3 as defined in SEQ ID NO: 124 and a variable light (VL) chain comprising CDR1 as defined in SEQ ID NO: 125, CDR2 comprising or consisting of the sequence of GAS and CDR3 as defined in SEQ ID NO: 126;
w) a variable heavy (VH) chain comprising CDR1 as defined in SEQ ID NO: 127, CDR2 as defined in SEQ ID NO: 128 and CDR3 as defined in SEQ ID NO: 129 and a variable light (VL) chain comprising CDR1 as defined in SEQ ID NO: 130, CDR2 comprising or consisting of the sequence of GAS and CDR3 as defined in SEQ ID NO: 131;
x) a variable heavy (VH) chain comprising CDR1 as defined in SEQ ID NO: 132, CDR2 as defined in SEQ ID NO: 133 and CDR3 as defined in SEQ ID NO: 134 and a variable light (VL) chain comprising CDR1 as defined in SEQ ID NO: 135, CDR2 comprising or consisting of the sequence of LGS and CDR3 as defined in SEQ ID NO: 136;
y) a variable heavy (VH) chain comprising CDR1 as defined in SEQ ID NO: 137, CDR2 as defined in SEQ ID NO: 138 and CDR3 as defined in SEQ ID NO: 139 and a variable light (VL) chain comprising CDR1 as defined in SEQ ID NO: 140, CDR2 comprising or consisting of the sequence of LGS and CDR3 as defined in SEQ ID NO: 141;
z) a variable heavy (VH) chain comprising CDR1 as defined in SEQ ID NO: 142, CDR2 as defined in SEQ ID NO: 143 and CDR3 as defined in SEQ ID NO: 144 and a variable light (VL) chain comprising CDR1 as defined in SEQ ID NO: 145, CDR2 comprising or consisting of the sequence of EGN and CDR3 as defined in SEQ ID NO: 146;
aa) a variable heavy (VH) chain comprising CDR1 as defined in SEQ ID NO: 147, CDR2 as defined in SEQ ID NO: 148 and CDR3 as defined in SEQ ID NO: 149 and a variable light (VL) chain comprising CDR1 as defined in SEQ ID NO: 150, CDR2 comprising or consisting of the sequence of GAS and CDR3 as defined in SEQ ID NO: 151;
ab) a variable heavy (VH) chain comprising CDR1 as defined in SEQ ID NO: 152, CDR2 as defined in SEQ ID NO: 153 and CDR3 as defined in SEQ ID NO: 154 and a variable light (VL) chain comprising CDR1 as defined in SEQ ID NO: 155, CDR2 comprising or consisting of the sequence of DVS and CDR3 as defined in SEQ ID NO: 156;
ac) a variable heavy (VH) chain comprising CDR1 as defined in SEQ ID NO: 157, CDR2 as defined in SEQ ID NO: 158 and CDR3 as defined in SEQ ID NO: 159 and a variable light (VL) chain comprising CDR1 as defined in SEQ ID NO: 160, CDR2 as defined in SEQ ID NO: 161 and CDR3 as defined in SEQ ID NO: 162;
ad) a variable heavy (VH) chain comprising CDR1 as defined in SEQ ID NO: 163, CDR2 as defined in SEQ ID NO: 164 and CDR3 as defined in SEQ ID NO: 165 and a variable light (VL) chain comprising CDR1 as defined in SEQ ID NO: 166, CDR2 comprising or consisting of the sequence of WAS and CDR3 as defined in SEQ ID NO: 167;
ae) a variable heavy (VH) chain comprising CDR1 as defined in SEQ ID NO: 168, CDR2 as defined in SEQ ID NO: 169 and CDR3 as defined in SEQ ID NO: 170 and a variable light (VL) chain comprising CDR1 as defined in SEQ ID NO: 171, CDR2 comprising or consisting of the sequence of EVS and CDR3 as defined in SEQ ID NO: 172;
af) a variable heavy (VH) chain comprising CDR1 as defined in SEQ ID NO: 304, CDR2 as defined in SEQ ID NO: 308 and CDR3 as defined in SEQ ID NO: 312 and a variable light (VL) chain comprising CDR1 as defined in SEQ ID NO: 316, CDR2 comprising or consisting of the sequence of GAS and CDR3 as defined in SEQ ID NO: 320;
ag) a variable heavy (VH) chain comprising CDR1 as defined in SEQ ID NO: 305, CDR2 as defined in SEQ ID NO: 309 and CDR3 as defined in SEQ ID NO: 313 and a variable light (VL) chain comprising CDR1 as defined in SEQ ID NO: 317, CDR2 comprising or consisting of the sequence of GAS and CDR3 as defined in SEQ ID NO: 321;
ah) a variable heavy (VH) chain comprising CDR1 as defined in SEQ ID NO: 306, CDR2 as defined in SEQ ID NO: 310 and CDR3 as defined in SEQ ID NO: 314 and a variable light (VL) chain comprising CDR1 as defined in SEQ ID NO: 318, CDR2 comprising or consisting of the sequence of GAS and CDR3 as defined in SEQ ID NO: 322;
ai) a variable heavy (VH) chain comprising CDR1 as defined in SEQ ID NO: 307, CDR2 as defined in SEQ ID NO: 311 and CDR3 as defined in SEQ ID NO: 315 and a variable light (VL) chain comprising CDR1 as defined in SEQ ID NO: 319, CDR2 comprising or consisting of the sequence of WAS and CDR3 as defined in SEQ ID NO: 323;
aj) a variable heavy (VH) chain comprising CDR1 as defined in SEQ ID NO: 332, CDR2 as defined in SEQ ID NO: 342 and CDR3 as defined in SEQ ID NO: 352 and a variable light (VL) chain comprising CDR1 as defined in SEQ ID NO: 362, CDR2 comprising or consisting of the sequence of SNN and CDR3 as defined in SEQ ID NO: 372;
ak) a variable heavy (VH) chain comprising CDR1 as defined in SEQ ID NO: 333, CDR2 as defined in SEQ ID NO: 343 and CDR3 as defined in SEQ ID NO: 353 and a variable light (VL) chain comprising CDR1 as defined in SEQ ID NO: 363, CDR2 comprising or consisting of the sequence of GAS and CDR3 as defined in SEQ ID NO: 373;
al) a variable heavy (VH) chain comprising CDR1 as defined in SEQ ID NO: 334, CDR2 as defined in SEQ ID NO: 344 and CDR3 as defined in SEQ ID NO: 354 and a variable light (VL) chain comprising CDR1 as defined in SEQ ID NO: 364, CDR2 comprising or consisting of the sequence of GAS and CDR3 as defined in SEQ ID NO: 374;
am) a variable heavy (VH) chain comprising CDR1 as defined in SEQ ID NO: 335, CDR2 as defined in SEQ ID NO: 345 and CDR3 as defined in SEQ ID NO: 355 and a variable light (VL) chain comprising CDR1 as defined in SEQ ID NO: 365, CDR2 comprising or consisting of the sequence of GAS and CDR3 as defined in SEQ ID NO: 375;
an) a variable heavy (VH) chain comprising CDR1 as defined in SEQ ID NO: 336, CDR2 as defined in SEQ ID NO: 346 and CDR3 as defined in SEQ ID NO: 356 and a variable light (VL) chain comprising CDR1 as defined in SEQ ID NO: 366, CDR2 comprising or consisting of the sequence of WAS and CDR3 as defined in SEQ ID NO: 376;
ao) a variable heavy (VH) chain comprising CDR1 as defined in SEQ ID NO: 337, CDR2 as defined in SEQ ID NO: 347 and CDR3 as defined in SEQ ID NO: 357 and a variable light (VL) chain comprising CDR1 as defined in SEQ ID NO: 367, CDR2 comprising or consisting of the sequence of AAS and CDR3 as defined in SEQ ID NO: 377;
ap) a variable heavy (VH) chain comprising CDR1 as defined in SEQ ID NO: 338, CDR2 as defined in SEQ ID NO: 348 and CDR3 as defined in SEQ ID NO: 358 and a variable light (VL) chain comprising CDR1 as defined in SEQ ID NO: 368, CDR2 comprising or consisting of the sequence of DAS and CDR3 as defined in SEQ ID NO: 378;
aq) a variable heavy (VH) chain comprising CDR1 as defined in SEQ ID NO: 339, CDR2 as defined in SEQ ID NO: 349 and CDR3 as defined in SEQ ID NO: 359 and a variable light (VL) chain comprising CDR1 as defined in SEQ ID NO: 369, CDR2 comprising or consisting of the sequence of DAS and CDR3 as defined in SEQ ID NO: 379;
ar) a variable heavy (VH) chain comprising CDR1 as defined in SEQ ID NO: 340, CDR2 as defined in SEQ ID NO: 350 and CDR3 as defined in SEQ ID NO: 360 and a variable light (VL) chain comprising CDR1 as defined in SEQ ID NO: 370, CDR2 comprising or consisting of the sequence of QDN and CDR3 as defined in SEQ ID NO: 380;
as) a variable heavy (VH) chain comprising CDR1 as defined in SEQ ID NO: 341, CDR2 as defined in SEQ ID NO: 351 and CDR3 as defined in SEQ ID NO: 361 and a variable light (VL) chain comprising CDR1 as defined in SEQ ID NO: 371, CDR2 comprising or consisting of the sequence of WAS and CDR3 as defined in SEQ ID NO: 381;
at) a variable heavy (VH) chain comprising CDR1 as defined in SEQ ID NO: 402, CDR2 as defined in SEQ ID NO: 412 and CDR3 as defined in SEQ ID NO: 422 and a variable light (VL) chain comprising CDR1 as defined in SEQ ID NO: 432, CDR2 comprising or consisting of the sequence of DAS and CDR3 as defined in SEQ ID NO: 442;
au) a variable heavy (VH) chain comprising CDR1 as defined in SEQ ID NO: 403, CDR2 as defined in SEQ ID NO: 413 and CDR3 as defined in SEQ ID NO: 423 and a variable light (VL) chain comprising CDR1 as defined in SEQ ID NO: 433, CDR2 comprising or consisting of the sequence of SSY and CDR3 as defined in SEQ ID NO: 443;
av) a variable heavy (VH) chain comprising CDR1 as defined in SEQ ID NO: 404, CDR2 as defined in SEQ ID NO: 414 and CDR3 as defined in SEQ ID NO: 424 and a variable light (VL) chain comprising CDR1 as defined in SEQ ID NO: 434, CDR2 comprising or consisting of the sequence of DDS and CDR3 as defined in SEQ ID NO: 444;
aw) a variable heavy (VH) chain comprising CDR1 as defined in SEQ ID NO: 405, CDR2 as defined in SEQ ID NO: 415 and CDR3 as defined in SEQ ID NO: 425 and a variable light (VL) chain comprising CDR1 as defined in SEQ ID NO: 435, CDR2 comprising or consisting of the sequence of ANS and CDR3 as defined in SEQ ID NO: 445;
ax) a variable heavy (VH) chain comprising CDR1 as defined in SEQ ID NO: 406, CDR2 as defined in SEQ ID NO: 416 and CDR3 as defined in SEQ ID NO: 426 and a variable light (VL) chain comprising CDR1 as defined in SEQ ID NO: 436, CDR2 comprising or consisting of the sequence of DAS and CDR3 as defined in SEQ ID NO: 446;
ay) a variable heavy (VH) chain comprising CDR1 as defined in SEQ ID NO: 407, CDR2 as defined in SEQ ID NO: 417 and CDR3 as defined in SEQ ID NO: 427 and a variable light (VL) chain comprising CDR1 as defined in SEQ ID NO: 437, CDR2 comprising or consisting of the sequence of DAS and CDR3 as defined in SEQ ID NO: 447;
az) a variable heavy (VH) chain comprising CDR1 as defined in SEQ ID NO: 408, CDR2 as defined in SEQ ID NO: 418 and CDR3 as defined in SEQ ID NO: 428 and a variable light (VL) chain comprising CDR1 as defined in SEQ ID NO: 438, CDR2 comprising or consisting of the sequence of DAS and CDR3 as defined in SEQ ID NO: 448;
ba) a variable heavy (VH) chain comprising CDR1 as defined in SEQ ID NO: 409, CDR2 as defined in SEQ ID NO: 419 and CDR3 as defined in SEQ ID NO: 429 and a variable light (VL) chain comprising CDR1 as defined in SEQ ID NO: 439, CDR2 comprising or consisting of the sequence of KVS and CDR3 as defined in SEQ ID NO: 449;
bb) a variable heavy (VH) chain comprising CDR1 as defined in SEQ ID NO: 410, CDR2 as defined in SEQ ID NO: 420 and CDR3 as defined in SEQ ID NO: 430 and a variable light (VL) chain comprising CDR1 as defined in SEQ ID NO: 440, CDR2 comprising or consisting of the sequence of AAS and CDR3 as defined in SEQ ID NO: 450; and
bc) a variable heavy (VH) chain comprising CDR1 as defined in SEQ ID NO: 411, CDR2 as defined in SEQ ID NO: 421 and CDR3 as defined in SEQ ID NO: 431 and a variable light (VL) chain comprising CDR1 as defined in SEQ ID NO: 441, CDR2 comprising or consisting of the sequence of DAS and CDR3 as defined in SEQ ID NO: 451.

14. A polynucleotide encoding an antibody, or an antigen-binding fragment thereof, according to claim 1.

15. A host cell comprising the polynucleotide of claim 14.

16. A method for producing an antibody comprising culturing the host cell of claim 15.

17. (canceled)

18. A pharmaceutical composition comprising at least two antibodies, or antigen-binding fragments thereof, wherein a first antibody, or antigen-binding fragment thereof is the antibody, or antigen-binding fragment thereof, of claim 1, and wherein a second antibody or antigen fragment thereof, specifically binds to RBD.

19. (canceled)

20. A method for treatment and/or prevention of a coronavirus infection in a subject, the method comprising delivering a therapeutically effective amount of the antibody, or antigen-binding fragment thereof, of claim 1 to a subject.

21. The method of claim 20, wherein the coronavirus is SARS-COV-2.

22. A method for identifying a coldspot antibody or an antigen-binding fragment thereof, the method comprising the steps of:
a) identifying a conserved region of an antigen;
b) generating a peptide comprising or consisting of the conserved region of the antigen or a fragment of the conserved region;
c) bringing at least two antibodies or antigen-binding fragments thereof of a convalescent subject into contact with the conserved region, wherein the convalescent subject is a subject that was previously exposed to the antigen; and
d) identifying at least one coldspot antibody or antigen-binding fragment thereof from the antibodies or antigen-binding fragments thereof of the convalescent subject, wherein the coldspot antibody or antigen-binding fragment thereof has at least one better coldspot binding property than at least one second antibody or antigen-binding fragments thereof of the antibodies of the convalescent subject, wherein a better coldspot binding property is a property selected from the group consisting of:
i) higher binding affinity to the peptide;
ii) cross-specificity to the peptide and to a second antigen, wherein the second antigen is a related antigen; and
iii) specific binding to the peptide and inhibition of a biologic activity of the antigen.

23. The method of claim 22, wherein identifying conserved regions in an antigen comprises comparison of a sequence of the antigen to a sequence of a related antigen.

24. The method of claim 22, wherein the conserved region comprises at least 17 consecutive aa with frequency of substitutions <0.1%.

25. The method of claim 22, wherein step c) comprises bringing at least two antibodies or antigen-binding fragments thereof of a convalescent subject into contact with the conserved region in an ELISA.

26. The method of claim 22, wherein step d) comprises fluorescence-activated cell sorting.

27. The method of claim 22, wherein the antibodies or antigen-binding fragments thereof of a convalescent subject are memory B cell-derived antibodies.

28. The method of claim 22, wherein identifying a conserved region further comprises identifying a secondary, tertiary and/or quaternary structure of the conserved region in the antigen.

29. The method of claim 22, wherein the antigen is part of a pathogen and wherein the convalescent subject is a subject that was previously exposed to the pathogen, a phylogenetically related pathogen, a vaccine to the pathogen and/or or an attenuated and/or inactivated version of the pathogen or to a phylogenetically related pathogen.

30. The method of claim 22, wherein the antigen is a cancer antigen.

31. An antibody, or antigen-binding fragment thereof, specifically binding to the RBD region of a coronavirus S protein, the antibody or antigen-binding fragment thereof comprising:
a) a variable heavy (VH) chain comprising CDR1 as defined in SEQ ID NO: 173, CDR2 as defined in SEQ ID NO: 174 and CDR3 as defined in SEQ ID NO: 175 and a variable light (VL) chain comprising CDR1 as defined in SEQ ID NO: 176, CDR2 comprising or consisting of the sequence of DAS and CDR3 as defined in SEQ ID NO: 177;
b) a variable heavy (VH) chain comprising CDR1 as defined in SEQ ID NO: 178, CDR2 as defined in SEQ ID NO: 179 and CDR3 as defined in SEQ ID NO: 180 and a variable light (VL) chain comprising CDR1 as defined in SEQ ID NO: 181, CDR2 comprising or consisting of the sequence of GAS and CDR3 as defined in SEQ ID NO: 182;
c) a variable heavy (VH) chain comprising CDR1 as defined in SEQ ID NO: 183, CDR2 as defined in SEQ ID NO: 184 and CDR3 as defined in SEQ ID NO: 185 and a variable light (VL) chain comprising CDR1 as defined in SEQ ID NO: 186, CDR2 comprising or consisting of the sequence of EVS and CDR3 as defined in SEQ ID NO: 187;
d) a variable heavy (VH) chain comprising CDR1 as defined in SEQ ID NO: 188, CDR2 as defined in SEQ ID NO: 189 and CDR3 as defined in SEQ ID NO: 190 and a variable light (VL) chain comprising CDR1 as defined in SEQ ID NO: 191, CDR2 comprising or consisting of the sequence of GNN and CDR3 as defined in SEQ ID NO: 192;
e) a variable heavy (VH) chain comprising CDR1 as defined in SEQ ID NO: 193, CDR2 as defined in SEQ ID NO: 194 and CDR3 as defined in SEQ ID NO: 195 and a variable light (VL) chain comprising CDR1 as defined in SEQ ID NO: 196, CDR2 comprising or consisting of the sequence of LGS and CDR3 as defined in SEQ ID NO: 197;
f) a variable heavy (VH) chain comprising CDR1 as defined in SEQ ID NO: 198, CDR2 as defined in SEQ ID NO: 199 and CDR3 as defined in SEQ ID NO: 200 and a variable light (VL) chain comprising CDR1 as defined in SEQ ID NO: 201, CDR2 comprising or consisting of the sequence of EDN and CDR3 as defined in SEQ ID NO: 202;
g) a variable heavy (VH) chain comprising CDR1 as defined in SEQ ID NO: 203, CDR2 as defined in SEQ ID NO: 204 and CDR3 as defined in SEQ ID NO: 205 and a variable light (VL) chain comprising CDR1 as defined in SEQ ID NO: 206, CDR2 comprising or consisting of the sequence of GAS and CDR3 as defined in SEQ ID NO: 207;
h) a variable heavy (VH) chain comprising CDR1 as defined in SEQ ID NO: 208, CDR2 as defined in SEQ ID NO: 209 and CDR3 as defined in SEQ ID NO: 210 and a variable light (VL) chain comprising CDR1 as defined in SEQ ID NO: 211, CDR2 comprising or consisting of the sequence of AAS and CDR3 as defined in SEQ ID NO: 212; or
i) a variable heavy (VH) chain comprising CDR1 as defined in SEQ ID NO: 213, CDR2 as defined in SEQ ID NO: 214 and CDR3 as defined in SEQ ID NO: 215 and a variable light (VL) chain comprising CDR1 as defined in SEQ ID NO: 216, CDR2 comprising or consisting of the sequence of GAS and CDR3 as defined in SEQ ID NO: 217.


SEQUENCE LISTING (471 sequences)

SEQ ID NO: 1 - protein, 25 aa, unidentified
  source 1..25: note = cs1 (FP) 814-838
KRSFIEDLLFNKVTLADAGFIKQYG

SEQ ID NO: 2 - protein, 20 aa, unidentified
  source 1..20: note = cs2 (HR2) 1141-1161
QPELDSFKEELDKYFKNHTS

SEQ ID NO: 3 - protein, 25 aa, unidentified
  source 1..25: note = cs3 113-137
KTQSLLIVNNATNVVIKVCEFQFCN

SEQ ID NO: 4 - protein, 19 aa, unidentified
  source 1..19: note = cs4 191-209
EFVFKNIDGYFKIYSKHTP

SEQ ID NO: 5 - protein, 30 aa, unidentified
  source 1..30: note = cs5 309-338
EKGIYQTSNFRVQPTESIVRFPNITNLCPF

SEQ ID NO: 6 - protein, 28 aa, unidentified
  source 1..28: note = cs6 377-404
FKCYGVSPTKLNDLCFTNVYADSFVIRG

SEQ ID NO: 7 - protein, 21 aa, unidentified
  source 1..21: note = cs7 418-438
IADYNYKLPDDFTGCVIAWNS

SEQ ID NO: 8 - protein, 23 aa, unidentified
  source 1..23: note = cs8 454-476
RLFRKSNLKPFERDISTEIYQAG

SEQ ID NO: 9 - protein, 24 aa, unidentified
  source 1..24: note = cs9 523-546
TVCGPKKSTNLVKNKCVNFNFNGL

SEQ ID NO: 10 - protein, 29 aa, unidentified
  source 1..29: note = cs10 584-612
ILDITPCSFGGVSVITPGTNTSNQVAVLY

SEQ ID NO: 11 - protein, 31 aa, unidentified
  source 1..31: note = cs11 733-763
KTSVDCTMYICGDSTECSNLLLQYGSFCTQL

SEQ ID NO: 12 - protein, 19 aa, unidentified
  source 1..19: note = cs12 860-878
VLPPLLTDEMIAQYTSALL

SEQ ID NO: 13 - protein, 40 aa, unidentified
  source 1..40: note = cs13 889-928
GAGAALQIPFAMQMAYRFNGIGVTQNVLYENQKLIANQFN

SEQ ID NO: 14 - protein, 37 aa, unidentified
  source 1..37: note = cs14 983-1019
RLDKVEAEVQIDRLITGRLQSLQTYVTQQLIRAAEIR

SEQ ID NO: 15 - protein, 43 aa, unidentified
  source 1..43: note = cs15 1028-1070
KMSECVLGQSKRVDFCGKGYHLMSFPQSAPHGVVFLHVTYVPA

SEQ ID NO: 16 - protein, 12 aa, unidentified
  source 1..12: note = SD1 sequence part 1
RVQPTESIVRFP

SEQ ID NO: 17 - protein, 8 aa, synthetic construct
  source 1..8: note = fp.001 VH CDR1
GFIFSNYA

SEQ ID NO: 18 - protein, 8 aa, synthetic construct
  source 1..8: note = fp.001 VH CDR2
ISFDARNK

SEQ ID NO: 19 - protein, 23 aa, synthetic construct
  source 1..23: note = fp.001 VH CDR3
ARDLFLSDYDNGVYDPTRGGFDY

SEQ ID NO: 20 - protein, 6 aa, synthetic construct
  source 1..6: note = fp.001 VL CDR1
QSVNNR

SEQ ID NO: 21 - protein, 10 aa, synthetic construct
  source 1..10: note = fp.001 VL CDR3
QQYHDWPLLS

SEQ ID NO: 22 - protein, 8 aa, synthetic construct
  source 1..8: note = fp.002 VH CDR1
GFNFDNYA

SEQ ID NO: 23 - protein, 8 aa, synthetic construct
  source 1..8: note = fp.002 VH CDR2
ISYEARNK

SEQ ID NO: 24 - protein, 23 aa, synthetic construct
  source 1..23: note = fp.002 VH CDR3
ARDLFLSDYDRSGYDPTRGGFDL

SEQ ID NO: 25 - protein, 6 aa, synthetic construct
  source 1..6: note = fp.002 VL CDR1
QSVSSS

SEQ ID NO: 26 - protein, 10 aa, synthetic construct
  source 1..10: note = fp.002 VL CDR3
QQYGDWPLVT

SEQ ID NO: 27 - protein, 8 aa, synthetic construct
  source 1..8: note = fp.003 VH CDR1
GFSFSNYA

SEQ ID NO: 28 - protein, 8 aa, synthetic construct
  source 1..8: note = fp.003 VH CDR2
ISFDTRNK

SEQ ID NO: 29 - protein, 23 aa, synthetic construct
  source 1..23: note = fp.003 VH CDR3
ARDLFLSDYDSRGYEPSRGGFDY

SEQ ID NO: 30 - protein, 6 aa, synthetic construct
  source 1..6: note = fp.003 VL CDR1
QSVSSS

SEQ ID NO: 31 - protein, 10 aa, synthetic construct
  source 1..10: note = fp.003 VL CDR3
QQYNDWPLLS

SEQ ID NO: 32 - protein, 8 aa, synthetic construct
  source 1..8: note = fp.004 VH CDR1
GFTFSNYA

SEQ ID NO: 33 - protein, 8 aa, synthetic construct
  source 1..8: note = fp.004 VH CDR2
ISFDTRNT

SEQ ID NO: 34 - protein, 23 aa, synthetic construct
ARDLFLSDYDSRGYDPITGGFDC

SEQ ID NO: 35 - protein, 6 aa, synthetic construct
  source 1..6: note = fp.004 VL CDR1
QSVGSR

SEQ ID NO: 36 - protein, 10 aa, synthetic construct
QQYNDWPLLT

SEQ ID NO: 37 - protein, 8 aa, synthetic construct
  source 1..8: note = fp.005 VH CDR1
GFRFNNYA

SEQ ID NO: 38 - protein, 8 aa, synthetic construct
  source 1..8: note = fp.005 VH CDR2
ISYEARNE

SEQ ID NO: 39 - protein, 23 aa, synthetic construct
  source 1..23: note = fp.005 VH CDR3
ARDLFLSDYDTSGYDRTRGGFDY

SEQ ID NO: 40 - protein, 6 aa, synthetic construct
  source 1..6: note = fp.005 VL CDR1
QSVSNG

SEQ ID NO: 41 - protein, 10 aa, synthetic construct
  source 1..10: note = fp.005 VL CDR3
QQYDDWPLIT

SEQ ID NO: 42 - protein, 8 aa, synthetic construct
  source 1..8: note = fp.006 VH CDR1
GFNFNNYA

SEQ ID NO: 43 - protein, 8 aa, synthetic construct
  source 1..8: note = fp.006 VH CDR2
ISYEARNK

SEQ ID NO: 44 - protein, 23 aa, synthetic construct
  source 1..23: note = fp.006 VH CDR3
ARDLFLSDYDRSGYDPTRGGFDH

SEQ ID NO: 45 - protein, 6 aa, synthetic construct
  source 1..6: note = fp.006 VL CDR1
QSVSSS

SEQ ID NO: 46 - protein, 10 aa, synthetic construct
  source 1..10: note = fp.006 VL CDR3
QQYGDWPLVT

SEQ ID NO: 47 - protein, 8 aa, synthetic construct
  source 1..8: note = fp.007 VH CDR1
GFIFSDAW

SEQ ID NO: 48 - protein, 10 aa, synthetic construct
  source 1..10: note = fp.007 VH CDR2
IKSKADGGTT

SEQ ID NO: 49 - protein, 18 aa, synthetic construct
  source 1..18: note = fp.007 VH CDR3
TTGRSRYCIGAVCYYFDH

SEQ ID NO: 50 - protein, 11 aa, synthetic construct
  source 1..11: note = fp.007 VL CDR1
QSLLHSNGYNY

SEQ ID NO: 51 - protein, 9 aa, synthetic construct
  source 1..9: note = fp.007 VL CDR3
TQTLQTPYT

SEQ ID NO: 52 - protein, 8 aa, synthetic construct
  source 1..8: note = fp.008 VH CDR1
GFRFNNYA

SEQ ID NO: 53 - protein, 8 aa, synthetic construct
  source 1..8: note = fp.008 VH CDR2
ISYEARNE

SEQ ID NO: 54 - protein, 23 aa, synthetic construct
  source 1..23: note = fp.008 VH CDR3
ARDVFLSDYDQSGYDVTRGGFDY

SEQ ID NO: 55 - protein, 6 aa, synthetic construct
  source 1..6: note = fp.008 VL CDR1
QSVSSS

SEQ ID NO: 56 - protein, 10 aa, synthetic construct
  source 1..10: note = fp.008 VL CDR3
QQYGDWPLVT

SEQ ID NO: 57 - protein, 8 aa, synthetic construct
  source 1..8: note = fp.009 VH CDR1
GFTFSNYA

SEQ ID NO: 58 - protein, 8 aa, synthetic construct
  source 1..8: note = fp.009 VH CDR2
ISFDTRNK

SEQ ID NO: 59 - protein, 23 aa, synthetic construct
  source 1..23: note = fp.009 VH CDR3
ARDVFLSDYDSRGFDRTRGGFDY

SEQ ID NO: 60 - protein, 6 aa, synthetic construct
  source 1..6: note = fp.009 VL CDR1
ESVGSS

SEQ ID NO: 61 - protein, 10 aa, synthetic construct
  source 1..10: note = fp.009 VL CDR3
QQYGDWPLLT

SEQ ID NO: 62 - protein, 8 aa, synthetic construct
  source 1..8: note = fp.010 VH CDR1
GFTFSTYA

SEQ ID NO: 63 - protein, 8 aa, synthetic construct
  source 1..8: note = fp.010 VH CDR2
ISSSGLST

SEQ ID NO: 64 - protein, 16 aa, synthetic construct
  source 1..16: note = fp.010 VH CDR3
AKVEYSGYDPRGPFDS

SEQ ID NO: 65 - protein, 6 aa, synthetic construct
  source 1..6: note = fp.010 VL CDR1
QSISGW

SEQ ID NO: 66 - protein, 10 aa, synthetic construct
  source 1..10: note = fp.010 VL CDR3
QEYSSYPLCT

SEQ ID NO: 67 - protein, 8 aa, synthetic construct
  source 1..8: note = fp.011 VH CDR1
GFTFNNYA

SEQ ID NO: 68 - protein, 8 aa, synthetic construct
  source 1..8: note = fp.011 VH CDR2
ISGSGGSS

SEQ ID NO: 69 - protein, 17 aa, synthetic construct
  source 1..17: note = fp.011 VH CDR3
AFIDYYSGWSYYYGLDV

SEQ ID NO: 70 - protein, 6 aa, synthetic construct
  source 1..6: note = fp.011 VL CDR1
QGVRTY

SEQ ID NO: 71 - protein, 10 aa, synthetic construct
  source 1..10: note = fp.011 VL CDR3
QQRYNWPLCS

SEQ ID NO: 72 - protein, 8 aa, synthetic construct
  source 1..8: note = hr2.012 VH CDR1
GYTFTSHY

SEQ ID NO: 73 - protein, 8 aa, synthetic construct
  source 1..8: note = hr2.012 VH CDR2
IKPSGGNT

SEQ ID NO: 74 - protein, 10 aa, synthetic construct
  source 1..10: note = hr2.012 VH CDR3
ARDQDHGLQR

SEQ ID NO: 75 - protein, 8 aa, synthetic construct
  source 1..8: note = hr2.012 VL CDR1
SSNIGNNF

SEQ ID NO: 76 - protein, 11 aa, synthetic construct
  source 1..11: note = hr2.012 VL CDR3
GTWDTSLGAWV

SEQ ID NO: 77 - protein, 8 aa, synthetic construct
  source 1..8: note = hr2.013 VH CDR1
GYTFTNNY

SEQ ID NO: 78 - protein, 8 aa, synthetic construct
  source 1..8: note = hr2.013 VH CDR2
IKPSSGST

SEQ ID NO: 79 - protein, 10 aa, synthetic construct
  source 1..10: note = hr2.013 VH CDR3
ARDQDHGLQN

SEQ ID NO: 80 - protein, 8 aa, synthetic construct
  source 1..8: note = hr2.013 VL CDR1
SSNIASNF

SEQ ID NO: 81 - protein, 11 aa, synthetic construct
  source 1..11: note = hr2.013 VL CDR3
GTWDTSLGAWV

SEQ ID NO: 82 - protein, 8 aa, synthetic construct
  source 1..8: note = hr2.014 VH CDR1
GYTFTTYA

SEQ ID NO: 83 - protein, 8 aa, synthetic construct
  source 1..8: note = hr2.014 VH CDR2
INAANGNT

SEQ ID NO: 84 - protein, 17 aa, synthetic construct
  source 1..17: note = hr2.014 VH CDR3
ARLLVVRDDPYYYGMDV

SEQ ID NO: 85 - protein, 12 aa, synthetic construct
  source 1..12: note = hr2.014 VL CDR1
QTVLYASSNKNY

SEQ ID NO: 86 - protein, 9 aa, synthetic construct
  source 1..9: note = hr2.014 VL CDR3
QQYYNSPIT

SEQ ID NO: 87 - protein, 8 aa, synthetic construct
  source 1..8: note = hr2.015 VH CDR1
GYTFSSYY

SEQ ID NO: 88 - protein, 8 aa, synthetic construct
  source 1..8: note = hr2.015 VH CDR2
INRSGSST

SEQ ID NO: 89 - protein, 9 aa, synthetic construct
  source 1..9: note = hr2.015 VH CDR3
ARDQDHALH

SEQ ID NO: 90 - protein, 8 aa, synthetic construct
  source 1..8: note = hr2.015 VL CDR1
SSNIGSNY

SEQ ID NO: 91 - protein, 11 aa, synthetic construct
  source 1..11: note = hr2.015 VL CDR3
GTWDTSLSAWV

SEQ ID NO: 92 - protein, 8 aa, synthetic construct
  source 1..8: note = hr2.016 VH CDR1
GFMFSKYA

SEQ ID NO: 93 - protein, 8 aa, synthetic construct
  source 1..8: note = hr2.016 VH CDR2
ISGGGGST

SEQ ID NO: 94 - protein, 11 aa, synthetic construct
  source 1..11: note = hr2.016 VH CDR3
AKLLVVFVIDY

SEQ ID NO: 95 - protein, 6 aa, synthetic construct
  source 1..6: note = hr2.016 VL CDR1
QSVSSN

SEQ ID NO: 96 - protein, 9 aa, synthetic construct
  source 1..9: note = hr2.016 VL CDR3
QQYGSSPPT

SEQ ID NO: 97 - protein, 8 aa, synthetic construct
  source 1..8: note = hr2.017 VH CDR1
GFAFSSSW

SEQ ID NO: 98 - protein, 8 aa, synthetic construct
  source 1..8: note = hr2.017 VH CDR2
INRDGSTT

SEQ ID NO: 99 - protein, 7 aa, synthetic construct
  source 1..7: note = hr2.017 VH CDR3
ATMGFDY

SEQ ID NO: 100 - protein, 12 aa, synthetic construct
  source 1..12: note = hr2.017 VL CDR1
QNVLYSSNNKNH

SEQ ID NO: 101 - protein, 9 aa, synthetic construct
  source 1..9: note = hr2.017 VL CDR3
QQYFSTPNT

SEQ ID NO: 102 - protein, 8 aa, synthetic construct
  source 1..8: note = hr2.018 VH CDR1
GSSVIDAW

SEQ ID NO: 103 - protein, 10 aa, synthetic construct
  source 1..10: note = hr2.018 VH CDR2
IKSKTNGATT

SEQ ID NO: 104 - protein, 10 aa, synthetic construct
  source 1..10: note = hr2.018 VH CDR3
TTSPLPNFDY

SEQ ID NO: 105 - protein, 12 aa, synthetic construct
  source 1..12: note = hr2.018 VL CDR1
QSVLYSSNNKNY

SEQ ID NO: 106 - protein, 9 aa, synthetic construct
  source 1..9: note = hr2.018 VL CDR3
QQYYSPAIT

SEQ ID NO: 107 - protein, 8 aa, synthetic construct
  source 1..8: note = hr2.019 VH CDR1
GSSVIDVW

SEQ ID NO: 108 - protein, 10 aa, synthetic construct
  source 1..10: note = hr2.019 VH CDR2
IKTKASGAAT

SEQ ID NO: 109 - protein, 10 aa, synthetic construct
  source 1..10: note = hr2.019 VH CDR3
TTSPLPNFDY

SEQ ID NO: 110 - protein, 12 aa, synthetic construct
  source 1..12: note = hr2.019 VL CDR1
QSVFYFANNKNY

SEQ ID NO: 111 - protein, 9 aa, synthetic construct
  source 1..9: note = hr2.019 VL CDR3
QQYSSPAIT

SEQ ID NO: 112 - protein, 8 aa, synthetic construct
  source 1..8: note = hr2.020 VH CDR1
GYRFASHW

SEQ ID NO: 113 - protein, 8 aa, synthetic construct
  source 1..8: note = hr2.020 VH CDR2
IYPGDSDT

SEQ ID NO: 114 - protein, 18 aa, synthetic construct
  source 1..18: note = hr2.020 VH CDR3
ARRPPHLDGGDSPIRFDH

SEQ ID NO: 115 - protein, 8 aa, synthetic construct
  source 1..8: note = hr2.020 VL CDR1
RSNIGSND

SEQ ID NO: 116 - protein, 10 aa, synthetic construct
  source 1..10: note = hr2.020 VL CDR3
ATWDDRLTGV

SEQ ID NO: 117 - protein, 8 aa, synthetic construct
  source 1..8: note = hr2.021 VH CDR1
EAIFSGSY

SEQ ID NO: 118 - protein, 8 aa, synthetic construct
  source 1..8: note = hr2.021 VH CDR2
ITPSGDKT

SEQ ID NO: 119 - protein, 11 aa, synthetic construct
  source 1..11: note = hr2.021 VH CDR3
ARQADKGGWDY

SEQ ID NO: 120 - protein, 7 aa, synthetic construct
  source 1..7: note = hr2.021 VL CDR1
QSVTSNY

SEQ ID NO: 121 - protein, 11 aa, synthetic construct
  source 1..11: note = hr2.021 VL CDR3
QQYGSSPPTFT

SEQ ID NO: 122 - protein, 8 aa, synthetic construct
  source 1..8: note = hr2.023 VH CDR1
ENIFSDSY

SEQ ID NO: 123 - protein, 8 aa, synthetic construct
  source 1..8: note = hr2.023 VH CDR2
ITPSGDKT

SEQ ID NO: 124 - protein, 11 aa, synthetic construct
  source 1..11: note = hr2.023 VH CDR3
ARQATKGGWDY

SEQ ID NO: 125 - protein, 7 aa, synthetic construct
  source 1..7: note = hr2.023 VL CDR1
QSVTRNY

SEQ ID NO: 126 - protein, 11 aa, synthetic construct
  source 1..11: note = hr2.023 VL CDR3
QQYGSSPPTFI

SEQ ID NO: 127 - protein, 8 aa, synthetic construct
  source 1..8: note = hr2.024 VH CDR1
EDIFTDSY

SEQ ID NO: 128 - protein, 8 aa, synthetic construct
  source 1..8: note = hr2.024 VH CDR2
ITPSGDKT

SEQ ID NO: 129 - protein, 11 aa, synthetic construct
  source 1..11: note = hr2.024 VH CDR3
ARQAEKGGWDY

SEQ ID NO: 130 - protein, 7 aa, synthetic construct
  source 1..7: note = hr2.024 VL CDR1
QTITSNY

SEQ ID NO: 131 - protein, 11 aa, synthetic construct
  source 1..11: note = hr2.024 VL CDR3
QQYGSSPPTFI

SEQ ID NO: 132 - protein, 8 aa, synthetic construct
  source 1..8: note = hr2.025 VH CDR1
GYSFTNYW

SEQ ID NO: 133 - protein, 8 aa, synthetic construct
  source 1..8: note = hr2.025 VH CDR2
IFPGDSDT

SEQ ID NO: 134 - protein, 17 aa, synthetic construct
  source 1..17: note = hr2.025 VH CDR3
ARRRGNSGNHDPYGMDV

SEQ ID NO: 135 - protein, 11 aa, synthetic construct
  source 1..11: note = hr2.025 VL CDR1
QSLLHSNGYNY

SEQ ID NO: 136 - protein, 9 aa, synthetic construct
  source 1..9: note = hr2.025 VL CDR3
MQALQTPWT

SEQ ID NO: 137 - protein, 8 aa, synthetic construct
  source 1..8: note = hr2.027 VH CDR1
GYSFANYW

SEQ ID NO: 138 - protein, 8 aa, synthetic construct
  source 1..8: note = hr2.027 VH CDR2
IFPGDSDT

SEQ ID NO: 139 - protein, 17 aa, synthetic construct
  source 1..17: note = hr2.027 VH CDR3
ARRRGNSGNHDPYGMDV

SEQ ID NO: 140 - protein, 11 aa, synthetic construct
  source 1..11: note = hr2.027 VL CDR1
QSLLHSNGYNY

SEQ ID NO: 141 - protein, 9 aa, synthetic construct
  source 1..9: note = hr2.027 VL CDR3
MQALETPWT

SEQ ID NO: 142 - protein, 8 aa, synthetic construct
  source 1..8: note = sd1.032 VH CDR1
GGTFNNYA

SEQ ID NO: 143 - protein, 8 aa, synthetic construct
  source 1..8: note = sd1.032 VH CDR2
IIPMLGTE

SEQ ID NO: 144 - protein, 18 aa, synthetic construct
  source 1..18: note = sd1.032 VH CDR3
ARLTVPTPHSYYSYGLDA

SEQ ID NO: 145 - protein, 9 aa, synthetic construct
  source 1..9: note = sd1.032 VL CDR1
SSDVGSYNL

SEQ ID NO: 146 - protein, 10 aa, synthetic construct
  source 1..10: note = sd1.032 VL CDR3
CSYAGSSTHV

SEQ ID NO: 147 - protein, 8 aa, synthetic construct
  source 1..8: note = sd1.033 VH CDR1
GGVYSTYA

SEQ ID NO: 148 - protein, 8 aa, synthetic construct
  source 1..8: note = sd1.033 VH CDR2
IIPMFGTA

SEQ ID NO: 149 - protein, 20 aa, synthetic construct
  source 1..20: note = sd1.033 VH CDR3
ASPSAPTYYYDSSRYYSFDY

SEQ ID NO: 150 - protein, 7 aa, synthetic construct
  source 1..7: note = sd1.033 VL CDR1
QSVSSNF

SEQ ID NO: 151 - protein, 9 aa, synthetic construct
  source 1..9: note = sd1.033 VL CDR3
QQYGSSRYS

SEQ ID NO: 152 - protein, 8 aa, synthetic construct
  source 1..8: note = sd1.034 VH CDR1
GFSFSNYG

SEQ ID NO: 153 - protein, 8 aa, synthetic construct
  source 1..8: note = sd1.034 VH CDR2
ISYDGSHK

SEQ ID NO: 154 - protein, 10 aa, synthetic construct
  source 1..10: note = sd1.034 VH CDR3
ATDQGGSLGY

SEQ ID NO: 155 - protein, 9 aa, synthetic construct
  source 1..9: note = sd1.034 VL CDR1
SGDVGGYNY

SEQ ID NO: 156 - protein, 11 aa, synthetic construct
  source 1..11: note = sd1.034 VL CDR3
SSYTSTSTSWV

SEQ ID NO: 157 - protein, 8 aa, synthetic construct
  source 1..8: note = sd1.039 VH CDR1
GYSFPTYW

SEQ ID NO: 158 - protein, 8 aa, synthetic construct
  source 1..8: note = sd1.039 VH CDR2
IYPGDSDT

SEQ ID NO: 159 - protein, 10 aa, synthetic construct
  source 1..10: note = sd1.039 VH CDR3
ARYGDSHIDS

SEQ ID NO: 160 - protein, 7 aa, synthetic construct
  source 1..7: note = sd1.039 VL CDR1
SGYSKYK

SEQ ID NO: 161 - protein, 8 aa, synthetic construct
  source 1..8: note = sd1.039 VHL CDR2
VGAGGIVG

SEQ ID NO: 162 - protein, 14 aa, synthetic construct
  source 1..14: note = sd1.039 VL CDR3
GADHGSGSNFAYVV

SEQ ID NO: 163 - protein, 8 aa, synthetic construct
  source 1..8: note = sd1.040 VH CDR1
GYTFTSFY

SEQ ID NO: 164 - protein, 8 aa, synthetic construct
  source 1..8: note = sd1.040 VH CDR2
IDPSGGST

SEQ ID NO: 165 - protein, 14 aa, synthetic construct
  source 1..14: note = sd1.040 VH CDR3
TRDGSAGDNSWFDP

SEQ ID NO: 166 - protein, 12 aa, synthetic construct
  source 1..12: note = sd1.040 VL CDR1
RNVLYSSNNKNY

SEQ ID NO: 167 - protein, 9 aa, synthetic construct
  source 1..9: note = sd1.040 VL CDR3
QQYYNTPYT

SEQ ID NO: 168 - protein, 8 aa, synthetic construct
  source 1..8: note = sd1.047 VH CDR1
GFPFKNYG

SEQ ID NO: 169 - protein, 8 aa, synthetic construct
  source 1..8: note = sd1.047 VH CDR2
ISYDGRLE

SEQ ID NO: 170 - protein, 20 aa, synthetic construct
  source 1..20: note = sd1.047 VH CDR3
AKDGVMGFCSGDDCTPPFDS

SEQ ID NO: 171 - protein, 9 aa, synthetic construct
  source 1..9: note = sd1.047 VL CDR1
SSDVGGYNY

SEQ ID NO: 172 - protein, 9 aa, synthetic construct
  source 1..9: note = sd1.047 VL CDR3
SSYAGSPYV

SEQ ID NO: 173 - protein, 8 aa, synthetic construct
  source 1..8: note = rbd.031 VH CDR1
GFTFKTYG

SEQ ID NO: 174 - protein, 8 aa, synthetic construct
  source 1..8: note = rbd.031 VH CDR2
ISYEGSNE

SEQ ID NO: 175 - protein, 17 aa, synthetic construct
  source 1..17: note = rbd.031 VH CDR3
AKGEYYYHSSTYYYLDY

SEQ ID NO: 176 - protein, 6 aa, synthetic construct
  source 1..6: note = rbd.031 VL CDR1
QSISSW

SEQ ID NO: 177 - protein, 8 aa, synthetic construct
  source 1..8: note = rbd.031 VL CDR3
QQYDTYWT

SEQ ID NO: 178 - protein, 8 aa, synthetic construct
  source 1..8: note = rbd.035 VH CDR1
GFPFNTFA

SEQ ID NO: 179 - protein, 8 aa, synthetic construct
  source 1..8: note = rbd.035 VH CDR2
ISSRGDYT

SEQ ID NO: 180 - protein, 16 aa, synthetic construct
  source 1..16: note = rbd.035 VH CDR3
AKGESGYDLYLYGLDL

SEQ ID NO: 181 - protein, 6 aa, synthetic construct
  source 1..6: note = rbd.035 VL CDR1
QNINNY

SEQ ID NO: 182 - protein, 10 aa, synthetic construct
  source 1..10: note = rbd.035 VL CDR3
QQNSPTPPWT

SEQ ID NO: 183 - protein, 8 aa, synthetic construct
  source 1..8: note = rbd.038 VH CDR1
GYTFSGYY

SEQ ID NO: 184 - protein, 8 aa, synthetic construct
  source 1..8: note = rbd.038 VH CDR2
ISPTSGGT

SEQ ID NO: 185 - protein, 25 aa, synthetic construct
  source 1..25: note = rbd.038 VH CDR3
AREPFPYCTSDICHSNYFYYYAMDV

SEQ ID NO: 186 - protein, 9 aa, synthetic construct
  source 1..9: note = rbd.038 VL CDR1
SSDLGDHNY

SEQ ID NO: 187 - protein, 9 aa, synthetic construct
  source 1..9: note = rbd.038 VL CDR3
SSYAGSLYV

SEQ ID NO: 188 - protein, 10 aa, synthetic construct
  source 1..10: note = rbd.042 VH CDR1
GGSISSGGYY

SEQ ID NO: 189 - protein, 7 aa, synthetic construct
  source 1..7: note = rbd.042 VH CDR2
IYSSGST

SEQ ID NO: 190 - protein, 11 aa, synthetic construct
  source 1..11: note = rbd.042 VH CDR3
ASGRVPYYFDY

SEQ ID NO: 191 - protein, 9 aa, synthetic construct
  source 1..9: note = rbd.042 VL CDR1
SSNIGAGFD

SEQ ID NO: 192 - protein, 12 aa, synthetic construct
  source 1..12: note = rbd.042 VL CDR3
QSYDSSLSGPYV

SEQ ID NO: 193 - protein, 8 aa, synthetic construct
  source 1..8: note = rbd.043 VH CDR1
GGSFSNYG

SEQ ID NO: 194 - protein, 8 aa, synthetic construct
  source 1..8: note = rbd.043 VH CDR2
IIPLFGPA

SEQ ID NO: 195 - protein, 27 aa, synthetic construct
  source 1..27: note = rbd.043 VH CDR3
ARGVEIVATIVGPLVREDHYYYYGMDV

SEQ ID NO: 196 - protein, 11 aa, synthetic construct
  source 1..11: note = rbd.043 VL CDR1
QSLLHSNGYNY

SEQ ID NO: 197 - protein, 9 aa, synthetic construct
  source 1..9: note = rbd.043 VL CDR3
MQALQTPGT

SEQ ID NO: 198 - protein, 8 aa, synthetic construct
  source 1..8: note = rbd.045 VH CDR1
GGAFRSFF

SEQ ID NO: 199 - protein, 7 aa, synthetic construct
  source 1..7: note = rbd.045 VH CDR2
IYYSGST

SEQ ID NO: 200 - protein, 12 aa, synthetic construct
  source 1..12: note = rbd.045 VH CDR3
ARSQWLRGNFDH

SEQ ID NO: 201 - protein, 8 aa, synthetic construct
  source 1..8: note = rbd.045 VL CDR1
SGYIASNY

SEQ ID NO: 202 - protein, 9 aa, synthetic construct
  source 1..9: note = rbd.045 VL CDR3
QSFDTAYAV

SEQ ID NO: 203 - protein, 8 aa, synthetic construct
  source 1..8: note = rbd.046 VH CDR1
GFTFSHYG

SEQ ID NO: 204 - protein, 8 aa, synthetic construct
  source 1..8: note = rbd.046 VH CDR2
ISYDGSNK

SEQ ID NO: 205 - protein, 16 aa, synthetic construct
  source 1..16: note = rbd.046 VH CDR3
AKGEWYYDIFTGYYPY

SEQ ID NO: 206 - protein, 7 aa, synthetic construct
  source 1..7: note = rbd.046 VL CDR1
QSVSSSY

SEQ ID NO: 207 - protein, 9 aa, synthetic construct
  source 1..9: note = rbd.046 VL CDR3
QQYGSSPRT

SEQ ID NO: 208 - protein, 8 aa, synthetic construct
  source 1..8: note = rbd.048 VH CDR1
GFTVSSNY

SEQ ID NO: 209 - protein, 7 aa, synthetic construct
  source 1..7: note = rbd.048 VH CDR2
IYSGGST

SEQ ID NO: 210 - protein, 11 aa, synthetic construct
  source 1..11: note = rbd.048 VH CDR3
ARDVVYYGMDV

SEQ ID NO: 211 - protein, 6 aa, synthetic construct
  source 1..6: note = rbd.048 VL CDR1
QSISSY

SEQ ID NO: 212 - protein, 10 aa, synthetic construct
  source 1..10: note = rbd.048 VL CDR3
QESYSNSPYT

SEQ ID NO: 213 - protein, 10 aa, synthetic construct
  source 1..10: note = rbd.049 VH CDR1
GGSISSKSDY

SEQ ID NO: 214 - protein, 7 aa, synthetic construct
  source 1..7: note = rbd.049 VH CDR2
IYYTGTT

SEQ ID NO: 215 - protein, 16 aa, synthetic construct
  source 1..16: note = rbd.049 VH CDR3
ARDPSIAVATTVYFDA

SEQ ID NO: 216 - protein, 7 aa, synthetic construct
  source 1..7: note = rbd.049 VL CDR1
ESVKSNY

SEQ ID NO: 217 - protein, 11 aa, synthetic construct
  source 1..11: note = rbd.049 VL CDR3
QQYHTSPPRYT

SEQ ID NO: 218 - protein, 129 aa, synthetic construct
  source 1..129: note = fp.001 variable heavy chain sequence
VQLVESGGGVVQPGRPLRLSCSGSGFIFSNYAMHWVRQAPGKGLEWVAIISFDARNKRYADSVEGRFTISRDNSKNILYLQMNSLRLEDSAVYFCARDLFLSDYDNGVYDPTRGGFDYWGQGTLVTVSS

SEQ ID NO: 219 - protein, 108 aa, synthetic construct
  source 1..108: note = fp.001 variable light (VL) chain sequence
EIVMTQSPATLSLSPGERATLSCRASQSVNNRLAWYQQKLGQPPRLLISGASTRATGIPARFTGSGSGTEFTLTISSLQSEDFAVYYCQQYHDWPLLSFGGGTKVEIK

SEQ ID NO: 220 - protein, 129 aa, synthetic construct
  source 1..129: note = fp.002 variable heavy chain sequence
VQLVESGGGVVQPGTSLRLSCAGSGFNFDNYAIHWVRQAPGQGLEWVAIISYEARNKKYAGSVTGRFSISRDNSKSMVYLQMDSLRGEDTAVYYCARDLFLSDYDRSGYDPTRGGFDLWGQGTLVTVSS

SEQ ID NO: 221 - protein, 108 aa, synthetic construct
  source 1..108: note = fp.002 variable light (VL) chain sequence
EIVMTQSPATLSVSPGERATLSCRASQSVSSSLAWYQHKLGQAPRLLIYGASTRATGIPGRFSGSGSGTEFTLTISSLQSEDVAIYYCQQYGDWPLVTFGGGTKVEIK

SEQ ID NO: 222 - protein, 129 aa, synthetic construct
  source 1..129: note = fp.003 variable heavy chain sequence
VQLVESGGGVVQPGTSLRLSCTASGFSFSNYAMHWVRQAPGKGLEWVAIISFDTRNKEFADSVKGRFSISRDNSKNSLNLQMNSLRPEDTGVYYCARDLFLSDYDSRGYEPSRGGFDYWGQGTLVTVSS

SEQ ID NO: 223 - protein, 108 aa, synthetic construct
  source 1..108: note = fp.003 variable light (VL) chain sequence
EIVMTQSPATLSVSPGERATLSCRASQSVSSSLAWYQQKLGQPPRLLIYGASTRATGIPARFSGSGSGTEFTLTISSLKSEDSAVYYCQQYNDWPLLSFGGGTKVEIK

SEQ ID NO: 224 - protein, 129 aa, synthetic construct
  source 1..129: note = fp.004 variable heavy chain sequence
VQLVESGGGVVQPGTSLRLSCVASGFTFSNYAMHWVRQAPGKGLEWVAIISFDTRNTASAASVKGRFNISRDNSKNSLYLQMNSLRPEDTGVYYCARDLFLSDYDSRGYDPITGGFDCWGQGTLVTVSS

SEQ ID NO: 225 - protein, 108 aa, synthetic construct
  source 1..108: note = fp.004 variable light (VL) chain sequence
EIVMTQSPAALSVSPGEGATLSCRASQSVGSRLAWYQQKLGRPPRLLIYGASTRATGIPARFSGSGSGTEFTLTISSLQSEDFAVYYCQQYNDWPLLTFGGGTKVKIK

SEQ ID NO: 226 - protein, 129 aa, synthetic construct
  source 1..129: note = fp.005 variable heavy chain sequence
VQLVESGGGVVQPGNSLRLSCAASGFRFNNYAMHWVRQAPGKGLEWVAIISYEARNEKYADSVKGRFSISRDNAKNTLYLQMNSLRADDTGVYYCARDLFLSDYDTSGYDRTRGGFDYWGQGALVTVSS

SEQ ID NO: 227 - protein, 108 aa, synthetic construct
  source 1..108: note = fp.005 variable light (VL) chain sequence
EIVMTQSPATLSVSPGERATLSCRASQSVSNGLAWYQHRFGQPPRLLIYGASTRATGIPTRFSGSGSGTEFTLTISSLQSEDFAVYYCQQYDDWPLITFGGGTKVEIK

SEQ ID NO: 228 - protein, 129 aa, synthetic construct
  source 1..129: note = fp.006 variable heavy chain sequence
VQLVESGGGVVQPGGSLRLSCAASGFNFNNYALHWVRQAPGKGLEWVAIISYEARNKKYGVSVMGRFSISRDNSKSIMYLEMNSLRVEDTGVYYCARDLFLSDYDRSGYDPTRGGFDHWGQGTLVTVSS

SEQ ID NO: 229 - protein, 108 aa, synthetic construct
  source 1..108: note = fp.006 variable light (VL) chain sequence
EIVMTQSPATLSVSPGERATLSCRASQSVSSSLAWYQHRFGQAPRLLISGASTRATGIPGRFSGSGSGTEFTLTISSLQSEDVAVYYCQQYGDWPLVTFGGGTKVEIK

SEQ ID NO: 230 - protein, 127 aa, synthetic construct
  source 1..127: note = fp.007 variable heavy chain sequence
EVQLVESGGGLVKPGGSLRLSCAGTGFIFSDAWMTWVRQAPGKGLEWVGRIKSKADGGTTDYAAPVKGRFTISRDDSKNTLYLEINSLKTEDTAVYYCTTGRSRYCIGAVCYYFDHWGQGTLVTVSS

SEQ ID NO: 231 - protein, 112 aa, synthetic construct
  source 1..112: note = fp.007 variable light (VL) chain sequence
DIVMTQSPLSLPVTPGEPASISCRSSQSLLHSNGYNYLDWYLQKPGQSPQLLIYLGSNRASGVPDRFSGSGSGTDFTLKISRVGAEDVGVYYCTQTLQTPYTFGQGTKLEIK

SEQ ID NO: 232 - protein, 129 aa, synthetic construct
  source 1..129: note = fp.008 variable heavy chain sequence
VQLVESGGGVVQPGRSLRLSCRASGFRFNNYAMHWVRQAPGKGLEWVAIISYEARNEKYAASVKGRFSISRDNAKNTLYLQMNSLRPEDTAIYYCARDVFLSDYDQSGYDVTRGGFDYWGQGTLVTVSS

SEQ ID NO: 233 - protein, 108 aa, synthetic construct
  source 1..108: note = fp.008 variable light (VL) chain sequence
EIVMTQSPATLSVSPGESATLSCRASQSVSSSLAWYQHKFGQAPRLLIYGASTRATGIPDRFSGSGSGMEFTLTISSLQSEDVAIYYCQQYGDWPLVTFGGGTKVEIK

SEQ ID NO: 234 - protein, 129 aa, synthetic construct
  source 1..129: note = fp.009 variable heavy chain sequence
VQLVESGGGVVQPGTSLTLSCEASGFTFSNYAMHWVRQAPGKGLEWVAIISFDTRNKKFAGSVQGRFSISRDNAKNSIYLQMNNLRPEDTGIYYCARDVFLSDYDSRGFDRTRGGFDYWGQGTLVTVSS

SEQ ID NO: 235 - protein, 108 aa, synthetic construct
  source 1..108: note = fp.009 variable light (VL) chain sequence
EIVMTQSPATLSVSPGEKATLSCRASESVGSSLAWYQQKLGQAPRLLIYGASTRATGIPARFSGSGSGAEFTLTISSLQSEDFAIYYCQQYGDWPLLTFGGGTKVEIK

SEQ ID NO: 236 - protein, 123 aa, synthetic construct
  source 1..123: note = fp.010 variable heavy chain sequence
EVQLLESGGGLVQPGGSLRLSCAASGFTFSTYAMNWVRQAPGKGLEWVSAISSSGLSTQYADSVRGRFTISRDTSKNTLYLHMNSLRAGDTAVYYCAKVEYSGYDPRGPFDSWGQGTLVTVSS

SEQ ID NO: 237 - protein, 108 aa, synthetic construct
  source 1..108: note = fp.010 variable light (VL) chain sequence
DIQMTQSPSTLSASVGDRVTITCRASQSISGWLAWYQQKPGRAPKLLIYKASSLERGVPSRFSGSGSGTEFTLTISSLQPDDFATYYCQEYSSYPLCTFGQGTKLEIK

SEQ ID NO: 238 - protein, 124 aa, synthetic construct
  source 1..124: note = fp.011 variable heavy chain sequence
EVQLLESGGGLVQPGGSLRLSCAGSGFTFNNYAMTWVRQAPGRGLEWVSAISGSGGSSHYADSVKGRFTITRDNSKNTLYLQMNSLRAEDTAVYYCAFIDYYSGWSYYYGLDVWGQGTTVTVSS

SEQ ID NO: 239 - protein, 108 aa, synthetic construct
  source 1..108: note = fp.011 variable light (VL) chain sequence
EIVLTQSPATLSLSPGERATLSCRASQGVRTYVAWYQQKPGQAPRLLIYDASNRATGIPARFSGSGSGTDFTLTISSLEPEDFAVYYCQQRYNWPLCSFGQGTKLEIK

SEQ ID NO: 240 - protein, 117 aa, synthetic construct
  source 1..117: note = hr2.012 variable heavy chain sequence
QVQLVQSGAEVKKPGASVKVSCKASGYTFTSHYIHWVRQAPGQGLEWMGIIKPSGGNTIYAQTFQGRVTMTRDTSTSTVYMELRSLTSDDTAVYYCARDQDHGLQRWGQGTLVTVSS

SEQ ID NO: 241 - protein, 110 aa, synthetic construct
  source 1..110: note = hr2.012 variable light (VL) chain sequence
QSVLTQPPSVSAPPGQKVTISCSGSSSNIGNNFVSWYQQLPGTAPKLLIYDNIERPSGIPDRFSGSRSGTSATLGITGLQTGDEADYYCGTWDTSLGAWVFGGGTKVTVL

SEQ ID NO: 242 - protein, 117 aa, synthetic construct
  source 1..117: note = hr2.013 variable heavy chain sequence
QVQLVQSGAEVKKPGASVKISCKASGYTFTNNYMNWVRQAPGQGLEWMGIIKPSSGSTIYAHKFQGRVTMTRDTSTSTVYMEVRSLTSEDTAVYYCARDQDHGLQNWGQGTLVTVSS

SEQ ID NO: 243 - protein, 110 aa, synthetic construct
  source 1..110: note = hr2.013 variable light (VL) chain sequence
QSVLTQPPSVSAASGQKVTIYCSGSSSNIASNFVSWYQQFPGTAPKLLIYDNNQRPSGIPDRFSGSRSGTSATLGITGLQTGDEADYYCGTWDTSLGAWVFGGGTKLTVL

SEQ ID NO: 244 - protein, 124 aa, synthetic construct
  source 1..124: note = hr2.014 variable heavy chain sequence
QVQLVQSGAEVKKPGASVKVSCKASGYTFTTYAVHWLRQVPGQRPEWIGWINAANGNTKYSEKFQGRVTITRDTSASTAYMDLRSLRSEDTAVYYCARLLVVRDDPYYYGMDVWGQGTTVTVSS

SEQ ID NO: 245 - protein, 113 aa, synthetic construct
  source 1..113: note = hr2.014 variable light (VL) chain sequence
DIVMTQSPDSLAVSLGERATINCKSSQTVLYASSNKNYLAWYQQKPGQPPKLLIYWASTRESGVPDRFSGSGSGTDFTLTISSLQAEDVAVYYCQQYYNSPITFGPGTKVDIK

SEQ ID NO: 246 - protein, 116 aa, synthetic construct
  source 1..116: note = hr2.015 variable heavy chain sequence
QVQLVQSGAEVKKPGASVKVSCKASGYTFSSYYVNWVRQAPGQGLEWLGIINRSGSSTIYAQKFQGRVTMTRDTSTSTVYLELSSLRSEDTAVYYCARDQDHALHWGQGTLVTVSS

SEQ ID NO: 247 - protein, 110 aa, synthetic construct
  source 1..110: note = hr2.015 variable light (VL) chain sequence
QSVLTQPPSVSAAPGQKVTISCSGSSSNIGSNYVSWYQQLPGTAPKLLIYDNSERPSGIPDRFSGSRSGTSATLGITGLQTGDEADYYCGTWDTSLSAWVFGGGTKLTVL

SEQ ID NO: 248 - protein, 118 aa, synthetic construct
  source 1..118: note = hr2.016 variable heavy chain sequence
EVQLLESGGGLVQPGGSLRLSCAASGFMFSKYAMTWVRQTPGKGLEWVSVISGGGGSTYYADSVKGRFTISRDNSKNTLYLQMNSLRAEDTAVYYCAKLLVVFVIDYWGQGALVTVSS

SEQ ID NO: 249 - protein, 107 aa, synthetic construct
  source 1..107: note = hr2.016 variable light (VL) chain sequence
EIVLTQSPGTLSLSPGERATLFCRASQSVSSNLAWYQQKPGRAPRLLISGASSRATGIPDRFSGSGSGTDFTLTISRLEPEDFAVYYCQQYGSSPPTFGQGTKVEIK

SEQ ID NO: 250 - protein, 114 aa, synthetic construct
  source 1..114: note = hr2.017 variable heavy chain sequence
EVQLVESGGGLVQPGGSLRLSCAVSGFAFSSSWMHWIRQAPGRGLVWVSRINRDGSTTDYADSVKGRFTISRDNAKATLYLQMNSLRAEDTAVYYCATMGFDYWGQGALVTVSS

SEQ ID NO: 251 - protein, 113 aa, synthetic construct
  source 1..113: note = hr2.017 variable light (VL) chain sequence
DIVMTQSPDSLAVSLGERATINCKSSQNVLYSSNNKNHLSWYQQKPGQPPKLLIYWASTRKSGVPDRFSGSGSGTDFTLTINSLQAEDVAVYYCQQYFSTPNTFGQGTKLEIK

SEQ ID NO: 252 - protein, 119 aa, synthetic construct
  source 1..119: note = hr2.018 variable heavy chain sequence
EVQLVESGGGLVKPGGSLRLSCVASGSSVIDAWMNWVRQAPGKGLEWVGRIKSKTNGATTDYAAPVKGRFTISRDDSKNLSYLQMNSLKTEDTGIYYCTTSPLPNFDYWGRGTLVTVSS

SEQ ID NO: 253 - protein, 113 aa, synthetic construct
  source 1..113: note = hr2.018 variable light (VL) chain sequence
DIVMTQSPDFLAVSLGERATINCKSSQSVLYSSNNKNYLAWYQQKPGQPPKLLIYWASKRESGVPDRFSGSGSGTDFTLTISSLQAEDVAVYYCQQYYSPAITFGPGTKVDIK

SEQ ID NO: 254 - protein, 119 aa, synthetic construct
  source 1..119: note = hr2.019 variable heavy chain sequence
EVQLVESGGGLVKPGGSLRLSCAASGSSVIDVWMNWVRQAPGKGLEWVGRIKTKASGAATDYGAPVKGRFTISRDDSKNLSYLQMNSLRAEDTGIYYCTTSPLPNFDYWGRGTLVTVSS

SEQ ID NO: 255 - protein, 113 aa, synthetic construct
  source 1..113: note = hr2.019 variable light (VL) chain sequence
DIVMTQSPDFLAVSLGETATINCRSSQSVFYFANNKNYLAWYQQKPGQPPKLLIYWASKRESGVPDRFSGSGSGTDFTLTISSLQAEDVATYYCQQYSSPAITFGPGTKVDIK

SEQ ID NO: 256 - protein, 125 aa, synthetic construct
  source 1..125: note = hr2.020 variable heavy chain sequence
EVQLVQSGAEVKKPGESLKISCKGSGYRFASHWIGWVRQMPGKGLQWMGIIYPGDSDTKYSPSFQGQVTISADKSINTVYLHWTSLKASDTAVYYCARRPPHLDGGDSPIRFDHWGQGTLVTVSS

SEQ ID NO: 257 - protein, 109 aa, synthetic construct
  source 1..109: note = hr2.020 variable light (VL) chain sequence
QSVLTQPPSASGTPGQRVTISCSGSRSNIGSNDVYWYQQFPGTAPKLLIYRNNQRPSGVPDRFSGSKSGTSASLAISGLRSEDEADYYCATWDDRLTGVFGGGTKLTVV

SEQ ID NO: 258 - protein, 118 aa, synthetic construct
  source 1..118: note = hr2.021 variable heavy chain sequence
QVQLVQSGAEVKKPGASVKVSCKASEAIFSGSYLHWVRQAPGQGLEWMGIITPSGDKTFYAQRFQGRLTMTRDTSTNTLHMHLSGLNSEDTAMYYCARQADKGGWDYWGQGTLVTVSS

SEQ ID NO: 259 - protein, 110 aa, synthetic construct
  source 1..110: note = hr2.021 variable light (VL) chain sequence
EIVLTQSPGTLSLSPGERATLSCRASQSVTSNYVAWYQQKPGQAPRLLIYGASTRATGIPDRFSGGGSGTDFTLTITRLEPEDFAVYYCQQYGSSPPTFTFGPGTKVDIK

SEQ ID NO: 260 - protein, 118 aa, synthetic construct
  source 1..118: note = hr2.023 variable heavy chain sequence
QVQLVQSGAEVKKPGASVKVSCRASENIFSDSYLHWVRQAPGQGLEWMGIITPSGDKTFYAQQFQGRLTMTRDTPTNTLHMDLRGLTSADTAIYYCARQATKGGWDYWGQGTLVTVSS

SEQ ID NO: 261 - protein, 110 aa, synthetic construct
  source 1..110: note = hr2.023 variable light (VL) chain sequence
EIVLTQSPGTLSLSPGESATLSCRASQSVTRNYLAWYQQQPGQAPRLLIYGASNRATGVPDRFSGSGSGTDFTLTISRLEPEDFAVYYCQQYGSSPPTFIFGPGTKVDIK

SEQ ID NO: 262 - protein, 118 aa, synthetic construct
  source 1..118: note = hr2.024 variable heavy chain sequence
QVQLVQSGAEVKKPGASVKVSCKASEDIFTDSYLHWVRQAPGQGLEWMGIITPSGDKTFYAQQFQGRLTMTRDPSTNTLYMFLSGLRSDDTAMYFCARQAEKGGWDYWGHGTLVTVSS

SEQ ID NO: 263 - protein, 110 aa, synthetic construct
  source 1..110: note = hr2.024 variable light (VL) chain sequence
EIVLTQSPGTLSLSPGQRATLSCRSSQTITSNYLAWYQQRPGQAPRLLIYGASIRATGIPDRFSGSGSGTDFTLTISRLEPEDFAVYYCQQYGSSPPTFIFGPGTKVDIK

SEQ ID NO: 264 - protein, 124 aa, synthetic construct
  source 1..124: note = hr2.025 variable heavy chain sequence
EVQLVQSGAEVKKPGESLKISCKGSGYSFTNYWIAWVRLMPGKGLEWMGFIFPGDSDTRYSPSFQGQVTISADKSISTAYLQWSSLKASDTAIYYCARRRGNSGNHDPYGMDVWGHGTTVTVSS

SEQ ID NO: 265 - protein, 112 aa, synthetic construct
  source 1..112: note = hr2.025 variable light (VL) chain sequence
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGIKVEIK

SEQ ID NO: 266 - protein, 124 aa, synthetic construct
  source 1..124: note = hr2.027 variable heavy chain sequence
EVQLVQSGAEVKKPGESLKISCKGSGYSFANYWIAWVRLMPGKGLEWMGFIFPGDSDTRYSPSFQGQVTISADKSITTAYLHWSSLKASDTAIYFCARRRGNSGNHDPYGMDVWGHGTTVTVSS

SEQ ID NO: 267 - protein, 112 aa, synthetic construct
  source 1..112: note = hr2.027 variable light (VL) chain sequence
DIVMTQSPLSLPVTPGEAASISCRSSQSLLHSNGYNYLDWYLQKPGQSPQLLIYLGSNRASGVPDRFSGSGSGTDFTLKISRVEAEDVGVYYCMQALETPWTFGQGTKVEIK

SEQ ID NO: 268 - protein, 125 aa, synthetic construct
  source 1..125: note = sd1.032 variable heavy chain sequence
QVQLVQSGAEVKRPGSSVKVSCKASGGTFNNYAMTWVRQAPGQGLEWMGGIIPMLGTENYAQKFQGRVTITADESTSTAHMELSSLRSEDTAVYYCARLTVPTPHSYYSYGLDAWGQGTTVTVSS

SEQ ID NO: 269 - protein, 110 aa, synthetic construct
  source 1..110: note = sd1.032 variable light (VL) chain sequence
QSALTQPASVSGSPGQSITISCTGTSSDVGSYNLVSWFQQHAGRAPKLMIYEGNKRPSGISNRFSGSKSGNTASLTISGLQAEDEADYYCCSYAGSSTHVFGTGTKVTVL

SEQ ID NO: 270 - protein, 127 aa, synthetic construct
  source 1..127: note = sd1.033 variable heavy chain sequence
QVQLVQSGAEVKKPGSSVKVSCKASGGVYSTYAVSWVRQAPGQGLEWIGGIIPMFGTANYAQNFQGRVTITADESTRTAYMEVSSLRSEDTAVYYCASPSAPTYYYDSSRYYSFDYWGQGTLVTVSS

SEQ ID NO: 271 - protein, 108 aa, synthetic construct
  source 1..108: note = sd1.033 variable light (VL) chain sequence
EIVLTQSPGTLSLSPGERATLSCRASQSVSSNFLAWFQQKPGQPPRLLIYGASSRATGIPDRFSGSGSGTDFTLTISRLVPEDFAVYFCQQYGSSRYSFGQGTKLEIK

SEQ ID NO: 272 - protein, 117 aa, synthetic construct
  source 1..117: note = sd1.034 variable heavy chain sequence
EVQLVESGGGVVQPGRSLRLSCAASGFSFSNYGMHWVRQAPGKGLEWVAVISYDGSHKSFADSVKGRFTISRDNSKNTLNLQMNSLRREDTAVYYCATDQGGSLGYWGQGTLVTVSS

SEQ ID NO: 273 - protein, 111 aa, synthetic construct
  source 1..111: note = sd1.034 variable light (VL) chain sequence
QSALTQPASVSGSPGQSITISCTGTSGDVGGYNYVSWYQHHPGKAPKLMIYDVSNRPSGVSNRFSGSKSGNTASLTISGLQAEDEADYYCSSYTSTSTSWVFGGGTKLTVL

SEQ ID NO: 274 - protein, 117 aa, synthetic construct
  source 1..117: note = sd1.039 variable heavy chain sequence
EVQLVQSGAEVKKPGESLKISCKASGYSFPTYWIGWVRQMPGKGLEWMGIIYPGDSDTRYSPSFQGQVTISADKSISTAYLQWSSLKASDTAIYYCARYGDSHIDSWGQGTLVTVSS

SEQ ID NO: 275 - protein, 117 aa, synthetic construct
  source 1..117: note = sd1.039 variable light (VL) chain sequence
QSVLTQPRSASASLGASVTLNCTLSSGYSKYKVEWYQQRPGKGPRFLMRVGAGGIVGSKGDGIPDRFSVLGSGLNRYLIIKNIQQEDESDYHCGADHGSGSNFAYVVFGGGTKLTVL

SEQ ID NO: 276 - protein, 121 aa, synthetic construct
  source 1..121: note = sd1.040 variable heavy chain sequence
QVQLVQSGAEVKKPGASVKVSCKASGYTFTSFYIHWVRQAPGQGLEWMGIIDPSGGSTSYPQKFQGRVTMTRDTSTSTVYMDLSSLRSEDTAVYYCTRDGSAGDNSWFDPWGQGTLVTVSS

SEQ ID NO: 277 - protein, 113 aa, synthetic construct
  source 1..113: note = sd1.040 variable light (VL) chain sequence
DIVMTQSPDSLAVSLGERATINCKSSRNVLYSSNNKNYLAWYQQKPGQPPKLLIYWASARESGVPDRFSGSGSGTDFTLTISSLQAEDVAVYYCQQYYNTPYTFGQGTKLEIK

SEQ ID NO: 278 - skipped

SEQ ID NO: 279 - protein, 109 aa, synthetic construct
  source 1..109: note = sd1.047 variable light (VL) chain sequence
QSALTQPASVSGSPGQSITISCTGTSSDVGGYNYVSWFQQYPGKAPQLLIYEVSNRPSGVSNRFSGSKSGNTASLTISGLQAEDEADYYCSSYAGSPYVFGIGTRVTVL

SEQ ID NO: 280 - protein, 124 aa, synthetic construct
  source 1..124: note = rbd.031 variable heavy chain sequence
EVQLVESGGGVVQPGRSLRLSCAASGFTFKTYGMHWVRQAPGKGLEWVAVISYEGSNEYYAESVKGRFTISRDNSKNTLYLHMNSLRAEDTAVYYCAKGEYYYHSSTYYYLDYWGRGTLVTVSS

SEQ ID NO: 281 - protein, 106 aa, synthetic construct
DIQMTQSPSTLSASVGDRVTITCRASQSISSWLAWYQQKPGKAPKVLIYDASSLQSGVPLRFSGSGSGTEFTLTISSLQPDDFATYYCQQYDTYWTFGQGTKVEIK

SEQ ID NO: 282 - protein, 123 aa, synthetic construct
  source 1..123: note = rbd.035 variable heavy chain sequence
EVQLLESGGGLIEPGGSLRLSCAASGFPFNTFAMTWVRQPPGKGLEWVATISSRGDYTFYADFVKGRFSISRDNANNALWLDMSGLRAEDTALYYCAKGESGYDLYLYGLDLWGQGTTVTVSS

SEQ ID NO: 283 - protein, 108 aa, synthetic construct
  source 1..108: note = rbd.035 variable light (VL) chain sequence
DIQMTQSPSSLSASVGDGVTITCRASQNINNYLSWYQKKAGKAPKLLIYGASTLHSGVPSRFSGSGSGTDFTLTISSLQPEDFASYYCQQNSPTPPWTFGQGTKVEIK

SEQ ID NO: 284 - protein, 132 aa, synthetic construct
  source 1..132: note = rbd.038 variable heavy chain sequence
QVQLVQSGAEVKKPGASVKVSCKASGYTFSGYYMHWVRQAPGQGLEWVGWISPTSGGTKYAQKFQGRVTMTRDTSITTVYMELNRLRSDDTAVYYCAREPFPYCTSDICHSNYFYYYAMDVWGQGTTVTVSS

SEQ ID NO: 285 - protein, 109 aa, synthetic construct
  source 1..109: note = rbd.038 variable light (VL) chain sequence
QSALTQPPSASGSPGQSVTISCTGSSSDLGDHNYVSWYQQHPGKAPKVMIYEVSKRPSGVPDRFSGSKSGNTASLTVSGLQAEDEADYYCSSYAGSLYVFGTGTKVTVL

SEQ ID NO: 286 - protein, 119 aa, synthetic construct
  source 1..119: note = rbd.042 variable heavy chain sequence
QVQLQESGPGLVKPSQTLSLTCTVSGGSISSGGYYWSWIRQHPGKGLEWIGYIYSSGSTYYNPSLKSRVTISVDTSKNQFSLKLSSVTAADTAVYYCASGRVPYYFDYWGQGTLVTVSS

SEQ ID NO: 287 - protein, 112 aa, synthetic construct
  source 1..112: note = rbd.042 variable light (VL) chain sequence
QSVLTQPPSVSGAPGQRVTISCTGSSSNIGAGFDVHWYQRLPGTAPKLLIYGNNIRPSGVPDRFSGSKSGTSASLAITGLQAEDEADYYCQSYDSSLSGPYVFGTGTKVTVL

SEQ ID NO: 288 - protein, 134 aa, synthetic construct
  source 1..134: note = rbd.043 variable heavy chain sequence
QVQLVQSGAEVKKPGSSVKVSCKVSGGSFSNYGISWVRQAPGQGLEWMGGIIPLFGPADYAQKVQGRVTITADKSTSTAYMELRSLRSEDTAVYFCARGVEIVATIVGPLVREDHYYYYGMDVWGQGTTVTVSS

SEQ ID NO: 289 - protein, 112 aa, synthetic construct
  source 1..112: note = rbd.043 variable light (VL) chain sequence
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPGTFGQGTKLEIK

SEQ ID NO: 290 - protein, 118 aa, synthetic construct
  source 1..118: note = rbd.045 variable heavy chain sequence
QVQLQESGPGLVKPSETLSLTCTVSGGAFRSFFWTWIRQPPGKGLEWIGNIYYSGSTNYNPSLKSRVTISQDMSENQFSLKLSSVTAADTAVYYCARSQWLRGNFDHWGQGILVTVSS

SEQ ID NO: 291 - protein, 110 aa, synthetic construct
  source 1..110: note = rbd.045 variable light (VL) chain sequence
NFMLTQPHSVSESPGKTVTISCTATSGYIASNYVQWYQQRPGSAPTTVIYEDNQRPSGVPDRFSGSIDSSSNSASLTISGLKPEDEADYYCQSFDTAYAVFGGGTQLTVL

SEQ ID NO: 292 - protein, 123 aa, synthetic construct
  source 1..123: note = rbd.046 variable heavy chain sequence
EVQLVESGGGVVQPGRSLRLSCAASGFTFSHYGMHWVRQAPGKGLEWVAVISYDGSNKYYADSVKGRFTISRDNSKSTLYLQMNSLRAEDTAVYYCAKGEWYYDIFTGYYPYWGQGTLVTVSS

SEQ ID NO: 293 - protein, 108 aa, synthetic construct
  source 1..108: note = rbd.046 variable light (VL) chain sequence
EIVLTQSPGTLSLSPGERATLSCRASQSVSSSYLAWYQQKPGQAPRLLISGASSRATGIPDRFSGSGSGTDFTLTISRLEPEDFAVYYCQQYGSSPRTFGQGTKVEIK

SEQ ID NO: 294 - protein, 117 aa, synthetic construct
  source 1..117: note = rbd.048 variable heavy chain sequence
EVQLVESGGGLVQPGGSLRLSCAASGFTVSSNYMTWVRQAPGKGLEWVSVIYSGGSTFYTDSVKGRFTISRDNSKNTLYLQMNSLRAEDTAVYYCARDVVYYGMDVWGQGTTVTVSS

SEQ ID NO: 295 - protein, 108 aa, synthetic construct
  source 1..108: note = rbd.048 variable light (VL) chain sequence
DIQMTQSPSSLSASVGDRVTITCRASQSISSYLNWYQQKPGKAPKLLIYAASSLQSGVPSRFSGSGSGTDFTLTISSLQPEDFATYYCQESYSNSPYTFGQGTKLEIK

SEQ ID NO: 296 - protein, 124 aa, synthetic construct
  source 1..124: note = rbd.049 variable heavy chain sequence
QLQLQESGPGLVKPSETLSLSCTVSGGSISSKSDYWGWIRQSPGKGLEWIGSIYYTGTTYYNPSLKSRVSISLDKSKNQFSLKLNSLTAADTALYYCARDPSIAVATTVYFDAWGQGTLVTVSS

SEQ ID NO: 297 - protein, 110 aa, synthetic construct
  source 1..110: note = rbd.049 variable light (VL) chain sequence
EIVLTQSPGTLSLSPGERATLSCRASESVKSNYLAWYQQKLGQPPRLLIFGASTRATGIPDRFSGSGSGTDFTLTISRLEPEDFAVYYCQQYHTSPPRYTFGQGTKLEIK

SEQ ID NO: 298 - protein, 62 aa, unidentified
  source 1..62: note = SD1 sequence part 2
STNLVKNKCVNFNFNGLTGTGVLTESNKKFLPFQQFGRDIADTTDAVRDPQTLEILDITPCS

SEQ ID NO: 299 - protein, 273 aa, unidentified
  source 1..273: note = SD1-RBD
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 300 - protein, 318 aa, synthetic construct
  source 1..318: note = mouse Ig Hc leader peptide_SD1-RBD_linker_avitag_8h_tag
MGWSCIILFLVATATGVHSRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSGGGGSGLNDIFEAQKIEWHEHHHHHH

SEQ ID NO: 301 - protein, 199 aa, unidentified
  source 1..199: note = RBD
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 302 - protein, 252 aa, synthetic construct
  source 1..252: note = IL2 leader peptide_RBD_linker_8h-tag_avitag
MYRMQLLSCIALSLALVTNSNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGGGGSGGGGSHHHHHHHHGLNDIFEAQKIEWHE

SEQ ID NO: 303 - protein, 20 aa, synthetic construct
  source 1..20: note = FP peptide
PSKRSFIEDLLFNKVTLADA

SEQ ID NO: 304 - protein, 8 aa, synthetic construct
  source 1..8: note = hr2.086 CDRH1
GFTFSSYA

SEQ ID NO: 305 - protein, 8 aa, synthetic construct
  source 1..8: note = hr2.090 CDRH1
GVTFSRYA

SEQ ID NO: 306 - protein, 8 aa, synthetic construct
  source 1..8: note = hr2.091 CDRH1
GFTFSGYA

SEQ ID NO: 307 - protein, 8 aa, synthetic construct
  source 1..8: note = hr2.094 CDRH1
GFTFSTFW

SEQ ID NO: 308 - protein, 8 aa, synthetic construct
  source 1..8: note = hr2.086 CDRH2
ISGGGGSA

SEQ ID NO: 309 - protein, 8 aa, synthetic construct
  source 1..8: note = hr2.090 CDRH2
ISGGGGGT

SEQ ID NO: 310 - protein, 8 aa, synthetic construct
  source 1..8: note = hr2.091 CDRH2
ISGRGGST

SEQ ID NO: 311 - protein, 8 aa, synthetic construct
  source 1..8: note = hr2.094 CDRH2
IKSDGSEK

SEQ ID NO: 312 - protein, 11 aa, synthetic construct
  source 1..11: note = hr2.086 CDRH3
AKIGMILVFDY

SEQ ID NO: 313 - protein, 14 aa, synthetic construct
  source 1..14: note = hr2.090 CDRH3
ATLLTVPGPYYFEY

SEQ ID NO: 314 - protein, 14 aa, synthetic construct
  source 1..14: note = hr2.091 CDRH3
AKIFSGSSCCVFDW

SEQ ID NO: 315 - protein, 7 aa, synthetic construct
  source 1..7: note = hr2.094 CDRH3
ATWFGLH

SEQ ID NO: 316 - protein, 7 aa, synthetic construct
  source 1..7: note = hr2.086 CDRL1
QSVSNNY

SEQ ID NO: 317 - protein, 7 aa, synthetic construct
  source 1..7: note = hr2.090 CDRL1
QSVSSSY

SEQ ID NO: 318 - protein, 7 aa, synthetic construct
  source 1..7: note = hr2.091 CDRL1
QSVTSSN

SEQ ID NO: 319 - protein, 12 aa, synthetic construct
  source 1..12: note = hr2.094 CDRL1
QTVLYSSNNKNY

SEQ ID NO: 320 - protein, 9 aa, synthetic construct
  source 1..9: note = hr2.086 CDRL3
QQYGSSPPT

SEQ ID NO: 321 - protein, 6 aa, synthetic construct
  source 1..6: note = hr2.090 CDRL3
QHSRFT

SEQ ID NO: 322 - protein, 10 aa, synthetic construct
  source 1..10: note = hr2.091 CDRL3
SSMVSPPGSL

SEQ ID NO: 323 - protein, 9 aa, synthetic construct
  source 1..9: note = hr2.094 CDRL3
QQYHRIPWT

SEQ ID NO: 324 - protein, 118 aa, synthetic construct
  source 1..118: note = hr2.086 VH
EVQLLESGGGLVQPGGYLRLSCAASGFTFSSYAMSWVRQAPGKGLEWVSVISGGGGSAYYADSVKGRFTISRDNSKNTLYLQMNSLRAEDTAVYYCAKIGMILVFDYWGQGTLVTVSS

SEQ ID NO: 325 - protein, 121 aa, synthetic construct
  source 1..121: note = hr2.090 VH
EVQLLESGGGLVQPGGSLRLSCAASGVTFSRYAMSWVRQAPGKGLEWVSTISGGGGGTYYADSVKGRFTISRDNSKNTLYLQMNSLRAEDTAVYYCATLLTVPGPYYFEYWGQGTLVTVSS

SEQ ID NO: 326 - protein, 121 aa, synthetic construct
  source 1..121: note = hr2.091 VH
EVQLLESGGGLVQPGGSLRLSCAASGFTFSGYAMSWVRQTPGKGLEWVSVISGRGGSTYYADSVKGRFTISRDNSKNTLYLQMNSLRAEDTAVYYCAKIFSGSSCCVFDWWGQGTPVTVSS

SEQ ID NO: 327 - protein, 114 aa, synthetic construct
  source 1..114: note = hr2.094 VH
EVQLVESGGGLVQPGGSLRLSCAASGFTFSTFWMTWVRQAPGKGLEWVANIKSDGSEKYYVDSVKGRFTISRDNAKNSLYLQMNSLRAEDTAVYYCATWFGLHWGQGTLVTVSS

SEQ ID NO: 328 - protein, 108 aa, synthetic construct
  source 1..108: note = hr2.086 VL
EIVLTQSPGTLSLSPGERATLSCRASQSVSNNYLAWYQQKPGQAPRLLISGASSRATGIPDRFSGSGSGTDFTLTISRLEPEDFAVYYCQQYGSSPPTFGQGTKVEIK

SEQ ID NO: 329 - protein, 105 aa, synthetic construct
  source 1..105: note = hr2.090 VL
EIVLTQSPGTLSLSPGERATLSCRASQSVSSSYLAWYQQKPGQAPRLLIYGASSRATGIPDRFSGSGSGTDFTLTISRLESEDFAVYYCQHSRFTFGPGTRVDIK

SEQ ID NO: 330 - protein, 109 aa, synthetic construct
  source 1..109: note = hr2.091 VL
EIVLTQSPGTLSLSPGERATLSCRASQSVTSSNLAWYQQRPGQAPRLLIYGASSRATASQTGSVAVGLGQTSLSPSADWSLKILQCITVSSMVSPPGSLSAEGPRWRSN

SEQ ID NO: 331 - protein, 113 aa, synthetic construct
  source 1..113: note = hr2.094 VL
DIVMTQSPDSLAVSLGERASINCKSSQTVLYSSNNKNYLAWYQQKPGQPPKLLIHWASTRDSGVPDRFSGSGSGTDFTLTISSLQAEDVAVYYCQQYHRIPWTFGRGTKVEIK

SEQ ID NO: 332 - protein, 10 aa, synthetic construct
  source 1..10: note = sd1.100 CDRH1
GDSITSGNYY

SEQ ID NO: 333 - protein, 8 aa, synthetic construct
  source 1..8: note = sd1.102 CDRH1
GGTFSSYA

SEQ ID NO: 334 - protein, 8 aa, synthetic construct
  source 1..8: note = sd1.103 CDRH1
GGTLNNYA

SEQ ID NO: 335 - protein, 8 aa, synthetic construct
  source 1..8: note = sd1.107 CDRH1
GGTFSSYA

SEQ ID NO: 336 - protein, 8 aa, synthetic construct
  source 1..8: note = sd1.108 CDRH1
GYTFTSYY

SEQ ID NO: 337 - protein, 8 aa, synthetic construct
  source 1..8: note = sd1.127 CDRH1
GFTFSHAW

SEQ ID NO: 338 - protein, 8 aa, synthetic construct
  source 1..8: note = sd1.129 CDRH1
GGAFSNYA

SEQ ID NO: 339 - protein, 8 aa, synthetic construct
  source 1..8: note = sd1.132 CDRH1
GGTFSSYA

SEQ ID NO: 340 - protein, 8 aa, synthetic construct
  source 1..8: note = sd1.150 CDRH1
GFTFSSHG

SEQ ID NO: 341 - protein, 8 aa, synthetic construct
  source 1..8: note = sd1.151 CDRH1
GFTFSNAW

SEQ ID NO: 342 - protein, 7 aa, synthetic construct
  source 1..7: note = sd1.100 CDRH2
IFHTGNT

SEQ ID NO: 343 - protein, 8 aa, synthetic construct
  source 1..8: note = sd1.102 CDRH2
IIPLYGTA

SEQ ID NO: 344 - protein, 8 aa, synthetic construct
  source 1..8: note = sd1.103 CDRH2
IIPMFGAA

SEQ ID NO: 345 - protein, 8 aa, synthetic construct
  source 1..8: note = sd1.107 CDRH2
IIPMFGTT

SEQ ID NO: 346 - protein, 8 aa, synthetic construct
  source 1..8: note = sd1.108 CDRH2
INPRGGST

SEQ ID NO: 347 - protein, 10 aa, synthetic construct
  source 1..10: note = sd1.127 CDRH2
IKTKTEGGTI

SEQ ID NO: 348 - protein, 8 aa, synthetic construct
  source 1..8: note = sd1.129 CDRH2
IIPIFGTT

SEQ ID NO: 349 - protein, 8 aa, synthetic construct
  source 1..8: note = sd1.132 CDRH2
IIPMFGAP

SEQ ID NO: 350 - protein, 8 aa, synthetic construct
  source 1..8: note = sd1.150 CDRH2
ISFDGRNK

SEQ ID NO: 351 - protein, 10 aa, synthetic construct
  source 1..10: note = sd1.151 CDRH2
IKTKPEGGTT

SEQ ID NO: 352 - protein, 14 aa, synthetic construct
  source 1..14: note = sd1.100 CDRH3
ARDHKRLEGDWFDP

SEQ ID NO: 353 - protein, 16 aa, synthetic construct
  source 1..16: note = sd1.102 CDRH3
VRFRGGDYYYYYGMDV

SEQ ID NO: 354 - protein, 19 aa, synthetic construct
  source 1..19: note = sd1.103 CDRH3
ARVVIPYLGAHYYHYGVDV

SEQ ID NO: 355 - protein, 23 aa, synthetic construct
  source 1..23: note = sd1.107 CDRH3
ARVTHLNWRRGYEVGRISGAFDI

SEQ ID NO: 356 - protein, 17 aa, synthetic construct
  source 1..17: note = sd1.108 CDRH3
AREHREDTAMDNQPSDS

SEQ ID NO: 357 - protein, 8 aa, synthetic construct
  source 1..8: note = sd1.127 CDRH3
TTHSSPDY

SEQ ID NO: 358 - protein, 19 aa, synthetic construct
  source 1..19: note = sd1.129 CDRH3
ARADAYSGSYYPSANWFDP

SEQ ID NO: 359 - protein, 23 aa, synthetic construct
  source 1..23: note = sd1.132 CDRH3
ARMEYSNSPGRGGYYFYSNGMDV

SEQ ID NO: 360 - protein, 19 aa, synthetic construct
  source 1..19: note = sd1.150 CDRH3
AKDHDPDGSGSYFSNWFDA

SEQ ID NO: 361 - protein, 8 aa, synthetic construct
  source 1..8: note = sd1.151 CDRH3
STFSSPDH

SEQ ID NO: 362 - protein, 8 aa, synthetic construct
  source 1..8: note = sd1.100 CDRL1
SSNIGSNT

SEQ ID NO: 363 - protein, 6 aa, synthetic construct
  source 1..6: note = sd1.102 CDRL1
QTVSTN

SEQ ID NO: 364 - protein, 7 aa, synthetic construct
  source 1..7: note = sd1.103 CDRL1
QSVTSNY

SEQ ID NO: 365 - protein, 7 aa, synthetic construct
  source 1..7: note = sd1.107 CDRL1
QSVSSSY

SEQ ID NO: 366 - protein, 12 aa, synthetic construct
  source 1..12: note = sd1.108 CDRL1
RSVFYSSNNKSY

SEQ ID NO: 367 - protein, 6 aa, synthetic construct
  source 1..6: note = sd1.127 CDRL1
QGIGNS

SEQ ID NO: 368 - protein, 6 aa, synthetic construct
QSVSNY

SEQ ID NO: 369 - protein, 6 aa, synthetic construct
  source 1..6: note = sd1.132 CDRL1
QSVSTY

SEQ ID NO: 370 - protein, 6 aa, synthetic construct
  source 1..6: note = sd1.150 CDRL1
KLGDKY

SEQ ID NO: 371 - protein, 12 aa, synthetic construct
  source 1..12: note = sd1.151 CDRL1
QSVFHSSNNKNY

SEQ ID NO: 372 - protein, 11 aa, synthetic construct
  source 1..11: note = sd1.100 CDRL3
AAFDDSLSGPV

SEQ ID NO: 373 - protein, 8 aa, synthetic construct
  source 1..8: note = sd1.102 CDRL3
HQYNSRRT

SEQ ID NO: 374 - protein, 9 aa, synthetic construct
  source 1..9: note = sd1.103 CDRL3
QQYGSSRYT

SEQ ID NO: 375 - protein, 9 aa, synthetic construct
  source 1..9: note = sd1.107 CDRL3
QQYGSSPFT

SEQ ID NO: 376 - protein, 9 aa, synthetic construct
  source 1..9: note = sd1.108 CDRL3
QQYYNTPYT

SEQ ID NO: 377 - protein, 9 aa, synthetic construct
  source 1..9: note = sd1.127 CDRL3
QRYNSAPLT

SEQ ID NO: 378 - protein, 11 aa, synthetic construct
  source 1..11: note = sd1.129 CDRL3
QQRQNWPPGWT

SEQ ID NO: 379 - protein, 9 aa, synthetic construct
  source 1..9: note = sd1.132 CDRL3
QQRTYWPLT

SEQ ID NO: 380 - protein, 9 aa, synthetic construct
  source 1..9: note = sd1.150 CDRL3
QAWDTYSGV

SEQ ID NO: 381 - protein, 9 aa, synthetic construct
  source 1..9: note = sd1.151 CDRL3
HQYYSTPFT

SEQ ID NO: 382 - protein, 122 aa, synthetic construct
  source 1..122: note = sd1.100 VH
QVQLQESGPGLVKPSQTLSLTCSVSGDSITSGNYYWSWIRQHPGKGLEWIGNIFHTGNTYYNPSLKSRVTISIDTSKNQFSLKLSSVTAADTALYYCARDHKRLEGDWFDPWGQGSLVTVSS

SEQ ID NO: 383 - protein, 123 aa, synthetic construct
  source 1..123: note = sd1.102 VH
QVQLVQSGAEVKKPGSSVKVSCKASGGTFSSYAISWVRQAPGQGLEWMGGIIPLYGTANYAQKFQGRVTITADESTSTTYMELSSLTSEDTAVYYCVRFRGGDYYYYYGMDVWGQGTTVTVSS

SEQ ID NO: 384 - protein, 126 aa, synthetic construct
  source 1..126: note = sd1.103 VH
QVQLVQSGAEVKKPGSSMKVSCKASGGTLNNYAIIWVRQAPGQGLEWMGGIIPMFGAANYAQNFQGRVTITADESTRTAYLELSSLRSEDTAMYYCARVVIPYLGAHYYHYGVDVWGQGTTVTVSS

SEQ ID NO: 385 - protein, 130 aa, synthetic construct
  source 1..130: note = sd1.107 VH
QVQLVQSGAEVKKPGSSVKVSCKASGGTFSSYAISWVRQAPGQGLEWMGGIIPMFGTTNYAQKFQGRVTITADESTSTAYMELSSLRSEDTAVYYCARVTHLNWRRGYEVGRISGAFDIWGQGTMVTVSS

SEQ ID NO: 386 - protein, 124 aa, synthetic construct
  source 1..124: note = sd1.108 VH
QVQLVQSGAEVKKPGASVKVSCKASGYTFTSYYIHWVRQTPGQGLEWMGIINPRGGSTSYPQKFQGRVSMTRDTSTSTVFMELSSLRSDDTAVYYCAREHREDTAMDNQPSDSWGQGTLVTVSS

SEQ ID NO: 387 - protein, 117 aa, synthetic construct
  source 1..117: note = sd1.127 VH
EVQLVESGGALVKPGGSLTLSCVASGFTFSHAWMTWVRQAPGKGLEWVGRIKTKTEGGTIDYAAPLRGRFTISRDDSKNTLYLRMNSLKIEDTAKYYCTTHSSPDYWGQGTLVTVSS

SEQ ID NO: 388 - protein, 126 aa, synthetic construct
  source 1..126: note = sd1.129 VH
QVQLVQSGAEVKRPGSSVKVSCKASGGAFSNYAISWVRQAPGQGLEWMGGIIPIFGTTNYAQRFQGRVTISADESTGTAYMELSSLRFADTAIYYCARADAYSGSYYPSANWFDPWGQGTLVTVSS

SEQ ID NO: 389 - protein, 130 aa, synthetic construct
  source 1..130: note = sd1.132 VH
QVQLVQSGTEVKKPGSSVKVSCKASGGTFSSYAISWVRQAPGQGLEWMGGIIPMFGAPNYAQKVQGRVTITADESTSTAYMELSSLRSEDTAVYYCARMEYSNSPGRGGYYFYSNGMDVWGQGTTVTVSS

SEQ ID NO: 390 - protein, 126 aa, synthetic construct
  source 1..126: note = sd1.150 VH
EVQLVESGGGVVQPGRSLRLSCAASGFTFSSHGMHWVRQAPGRGLEWVAVISFDGRNKFYVDSVKGRFTISRDNSKNTLYLHMNSLRAADTAVYYCAKDHDPDGSGSYFSNWFDAWGQGTLVTVSS

SEQ ID NO: 391 - protein, 117 aa, synthetic construct
  source 1..117: note = sd1.151 VH
EVQLVESGGGLVKPGGSLRLSCAVSGFTFSNAWMTWVRQAPGKGLEWVGRIKTKPEGGTTDYATPVKGRFTISRDDSIDTLYLQMNSLKTEDTAVYYCSTFSSPDHWGQGTLVTVSS

SEQ ID NO: 392 - protein, 110 aa, synthetic construct
  source 1..110: note = sd1.100 VL
QSVLTQPPSASGTPGQRVTISCSGSSSNIGSNTVNWYLQLPGTAPKLLIYSNNLRPSGVPDRFSGSKSGTSASLAISGLQSGDEADYFCAAFDDSLSGPVFGGGTKLTVL

SEQ ID NO: 393 - protein, 106 aa, synthetic construct
  source 1..106: note = sd1.102 VL
EIVMTQSPATLSVSPGESATLSCRASQTVSTNLAWYQQKPGQAPRLLIFGASTRATGIPARFSGSGSGTEFTLSISSLQSEDFAVYYCHQYNSRRTFGQGTKLEIK

SEQ ID NO: 394 - protein, 108 aa, synthetic construct
  source 1..108: note = sd1.103 VL
EIVLTQSPGTLSLSPGERATLSCRASQSVTSNYLAWYQQKPGQAPRLLIYGASSRAAGIPDRFNGSGSGTDFTLTISRLEPEDFAVYYCQQYGSSRYTFGQGTKLEIK

SEQ ID NO: 395 - protein, 108 aa, synthetic construct
  source 1..108: note = sd1.107 VL
EIVLTQSPGTLSLSPGERATLSCRASQSVSSSYLAWYQQKPGQAPRLLIYGASSRATGIPDRFSGSGSGTDFTLTISRLEPEDFAVFYCQQYGSSPFTFGPGTKVDIK

SEQ ID NO: 396 - protein, 113 aa, synthetic construct
  source 1..113: note = sd1.108 VL
DIVMTQSPDSLSVSLGERATINCKSSRSVFYSSNNKSYLAWYQQKPGQPPKLLIYWASTRESGVRDRFSGSGSGTDFTLTISSLQAEDVAVYYCQQYYNTPYTFGQGTKLEIK

SEQ ID NO: 397 - protein, 107 aa, synthetic construct
  source 1..107: note = sd1.127 VL
DIQMTQSPSSLSASIGDRVTITCRASQGIGNSLLWYQQKRGKVPKLLIYAASTLQSGVPSRFSGSGSGTDFTLTISSLQPEDVATYYCQRYNSAPLTFGGGTKVEIK

SEQ ID NO: 398 - protein, 109 aa, synthetic construct
  source 1..109: note = sd1.129 VL
EIVLTQSPATLSLSPGERATLSCRASQSVSNYLAWYQQKPGQAPRLLIYDASNRATGIPARFSGSGSGTDFTLTISSLEPEDFAVYYCQQRQNWPPGWTFGQGTKVEIK

SEQ ID NO: 399 - protein, 107 aa, synthetic construct
  source 1..107: note = sd1.132 VL
EIVLTQSPATLSLSPGERATLSCRASQSVSTYLAWLQQKPGQAPRLLMYDASNRASGIPARFSGSGSGTEFTLTISSLEPEDFAVYYCQQRTYWPLTFGGGTKVEIK

SEQ ID NO: 400 - protein, 106 aa, synthetic construct
  source 1..106: note = sd1.150 VL
SYELTQPPSVSVSPGQTASITCSGDKLGDKYAYWYRQKPGQSPVVVIYQDNKRPSGIPERFSGSNSGNTATLTISGTQAMDEADYYCQAWDTYSGVFGTGTQVTVL

SEQ ID NO: 401 - protein, 113 aa, synthetic construct
  source 1..113: note = sd1.151 VL
DIVMTQSPDSLAVSLGERATINCKSSQSVFHSSNNKNYLIWYQHKPGQPPKLLISWASTRESGVPDRFSGSGSGTDFTLTISSLQAEDVAVYYCHQYYSTPFTFGPGTKVDIK

SEQ ID NO: 402 - protein, 10 aa, synthetic construct
  source 1..10: note = rbd.104 CDRH1
GVSVTSGSYF

SEQ ID NO: 403 - protein, 8 aa, synthetic construct
  source 1..8: note = rbd.106 CDRH1
GYSFYNHW

SEQ ID NO: 404 - protein, 8 aa, synthetic construct
  source 1..8: note = rbd.110 CDRH1
GFTFSRYG

SEQ ID NO: 405 - protein, 8 aa, synthetic construct
  source 1..8: note = rbd.112 CDRH1
GYSFTTYW

SEQ ID NO: 406 - protein, 8 aa, synthetic construct
  source 1..8: note = rbd.113 CDRH1
GYSFTNYW

SEQ ID NO: 407 - protein, 8 aa, synthetic construct
  source 1..8: note = rbd.117 CDRH1
GFTFSRYA

SEQ ID NO: 408 - protein, 8 aa, synthetic construct
  source 1..8: note = rbd.123 CDRH1
GGAFRMYA

SEQ ID NO: 409 - protein, 8 aa, synthetic construct
  source 1..8: note = rbd.141 CDRH1
GFTFSSYG

SEQ ID NO: 410 - protein, 8 aa, synthetic construct
  source 1..8: note = rbd.142 CDRH1
GLTFSRYG

SEQ ID NO: 411 - protein, 8 aa, synthetic construct
  source 1..8: note = rbd.143 CDRH1
GFTFRRYA

SEQ ID NO: 412 - protein, 7 aa, synthetic construct
  source 1..7: note = rbd.104 CDRH2
VHNSGST

SEQ ID NO: 413 - protein, 8 aa, synthetic construct
  source 1..8: note = rbd.106 CDRH2
IYPYDSDT

SEQ ID NO: 414 - protein, 8 aa, synthetic construct
  source 1..8: note = rbd.110 CDRH2
ILYDGSDQ

SEQ ID NO: 415 - protein, 8 aa, synthetic construct
  source 1..8: note = rbd.112 CDRH2
IYPGHSDA

SEQ ID NO: 416 - protein, 8 aa, synthetic construct
  source 1..8: note = rbd.113 CDRH2
IYVGDSDT

SEQ ID NO: 417 - protein, 8 aa, synthetic construct
  source 1..8: note = rbd.117 CDRH2
LSGSGDST

SEQ ID NO: 418 - protein, 8 aa, synthetic construct
  source 1..8: note = rbd.123 CDRH2
IIPISGTA

SEQ ID NO: 419 - protein, 8 aa, synthetic construct
  source 1..8: note = rbd.141 CDRH2
ISYDGKIK

SEQ ID NO: 420 - protein, 8 aa, synthetic construct
  source 1..8: note = rbd.142 CDRH2
TTYDGSNK

SEQ ID NO: 421 - protein, 8 aa, synthetic construct
  source 1..8: note = rbd.143 CDRH2
ISSDGDNT

SEQ ID NO: 422 - protein, 13 aa, synthetic construct
  source 1..13: note = rbd.104 CDRH3
ARLFCGGDCPEDY

SEQ ID NO: 423 - protein, 15 aa, synthetic construct
  source 1..15: note = rbd.106 CDRH3
ARRSDYGEFGGHSDY

SEQ ID NO: 424 - protein, 17 aa, synthetic construct
  source 1..17: note = rbd.110 CDRH3
AKTSYGYAYNFRLYFDY

SEQ ID NO: 425 - protein, 21 aa, synthetic construct
  source 1..21: note = rbd.112 CDRH3
ARLSYYDVSNFYYYDSYWFDS

SEQ ID NO: 426 - protein, 13 aa, synthetic construct
  source 1..13: note = rbd.113 CDRH3
ARQRCGGDCPFDL

SEQ ID NO: 427 - protein, 13 aa, synthetic construct
  source 1..13: note = rbd.117 CDRH3
AKGSRFGIDYFDC

SEQ ID NO: 428 - protein, 13 aa, synthetic construct
  source 1..13: note = rbd.123 CDRH3
ARGPRTGYDYFDF

SEQ ID NO: 429 - protein, 19 aa, synthetic construct
  source 1..19: note = rbd.141 CDRH3
AKYGIVTSFGVVPPYDMDV

SEQ ID NO: 430 - protein, 22 aa, synthetic construct
  source 1..22: note = rbd.142 CDRH3
AKVGGGEYCSGGSCYGSYGMDV

SEQ ID NO: 431 - protein, 13 aa, synthetic construct
  source 1..13: note = rbd.143 CDRH3
VKDPFSNFHYFDC

SEQ ID NO: 432 - protein, 6 aa, synthetic construct
  source 1..6: note = rbd.104 CDRL1
QSISSW

SEQ ID NO: 433 - protein, 8 aa, synthetic construct
  source 1..8: note = rbd.106 CDRL1
SSNIGSNS

SEQ ID NO: 434 - protein, 6 aa, synthetic construct
  source 1..6: note = rbd.110 CDRL1
NIGSAG

SEQ ID NO: 435 - protein, 9 aa, synthetic construct
  source 1..9: note = rbd.112 CDRL1
SSNLGAGYD

SEQ ID NO: 436 - protein, 6 aa, synthetic construct
  source 1..6: note = rbd.113 CDRL1
QSINRW

SEQ ID NO: 437 - protein, 6 aa, synthetic construct
  source 1..6: note = rbd.117 CDRL1
QSISSW

SEQ ID NO: 438 - protein, 6 aa, synthetic construct
  source 1..6: note = rbd.123 CDRL1
QSISSW

SEQ ID NO: 439 - protein, 11 aa, synthetic construct
  source 1..11: note = rbd.141 CDRL1
QSLVYSDGNTF

SEQ ID NO: 440 - protein, 6 aa, synthetic construct
  source 1..6: note = rbd.142 CDRL1
QSISSY

SEQ ID NO: 441 - protein, 6 aa, synthetic construct
  source 1..6: note = rbd.143 CDRL1
QSIRSW

SEQ ID NO: 442 - protein, 8 aa, synthetic construct
  source 1..8: note = rbd.104 CDRL3
QQYNSYKT

SEQ ID NO: 443 - protein, 11 aa, synthetic construct
  source 1..11: note = rbd.106 CDRL3
AAWDDSLNGVV

SEQ ID NO: 444 - protein, 11 aa, synthetic construct
  source 1..11: note = rbd.110 CDRL3
QVWDSNSAPVI

SEQ ID NO: 445 - protein, 11 aa, synthetic construct
  source 1..11: note = rbd.112 CDRL3
QSYDSSLGGYV

SEQ ID NO: 446 - protein, 8 aa, synthetic construct
  source 1..8: note = rbd.113 CDRL3
QQYNSYWT

SEQ ID NO: 447 - protein, 9 aa, synthetic construct
  source 1..9: note = rbd.117 CDRL3
QQYNTYPFS

SEQ ID NO: 448 - protein, 9 aa, synthetic construct
  source 1..9: note = rbd.123 CDRL3
QQYKSYPYT

SEQ ID NO: 449 - protein, 9 aa, synthetic construct
  source 1..9: note = rbd.141 CDRL3
MQGTNWPPT

SEQ ID NO: 450 - protein, 9 aa, synthetic construct
  source 1..9: note = rbd.142 CDRL3
QQSYSTPYT

SEQ ID NO: 451 - protein, 9 aa, synthetic construct
  source 1..9: note = rbd.143 CDRL3
QQYNSYSST

SEQ ID NO: 452 - protein, 121 aa, synthetic construct
  source 1..121: note = rbd.104 VH
QVQLQESGPGLVKPSETLSLTCTVSGVSVTSGSYFWSWIRQPPGKGLEWIGHVHNSGSTRYNPSLKSRVNIAVDTSKNQLSLKLSFVTAADTAVYYCARLFCGGDCPEDYWGQGTLVTVSS

SEQ ID NO: 453 - protein, 122 aa, synthetic construct
  source 1..122: note = rbd.106 VH
EVQLVQSGAEVKKPGESLKISCKGSGYSFYNHWIAWVRQMPGKGLEWMGIIYPYDSDTRYSPSFQGQVTISADKSITTAYLQWSSLKASDTAIYYCARRSDYGEFGGHSDYWGQGTLVTVSS

SEQ ID NO: 454 - protein, 124 aa, synthetic construct
  source 1..124: note = rbd.110 VH
EVQLVESGGGVVQPGRSLRLSCVASGFTFSRYGMHWVRQAPGKGLEWVATILYDGSDQYYAESVKGRFTISRDNSKNTLYLQMNSLREADTAVYYCAKTSYGYAYNFRLYFDYWGQGTLVTVSS

SEQ ID NO: 455 - protein, 128 aa, synthetic construct
  source 1..128: note = rbd.112 VH
EVQLVQSGAEVKKSGESLKISCKSSGYSFTTYWIAWVRQMPGKGLEWMGIIYPGHSDARYSPSFQGHVTISADKSISTAYLQWRSLKASDTAIYYCARLSYYDVSNFYYYDSYWFDSWGLGTLVTVSS

SEQ ID NO: 456 - protein, 120 aa, synthetic construct
  source 1..120: note = rbd.113 VH
EVQLVQSGAAVKKPGESLKISCKGSGYSFTNYWIGWVRQMPGKGLELMGIIYVGDSDTRYSPSFQGQVTISADKSITTAYLQWSSLKASDSAIYYCARQRCGGDCPFDLWGRGTLVTVSS

SEQ ID NO: 457 - protein, 120 aa, synthetic construct
  source 1..120: note = rbd.117 VH
EVQLVESGGGLVQPGGSLRLSCAASGFTFSRYAMSWVRQAPGKGLEWVSGLSGSGDSTYYADSVKGRFTISRDNSKKTLNLQMNSLRPEDTAVYYCAKGSRFGIDYFDCWGQGTLVTVSS

SEQ ID NO: 458 - protein, 120 aa, synthetic construct
  source 1..120: note = rbd.123 VH
QVQLVQSGAEVKKPGSSVKVSCKASGGAFRMYAISWVRQAPGQGLEWMGGIIPISGTATYTQNFQGRVTITVDRSTSTAYMELSSLRSDDTALYYCARGPRTGYDYFDFWGQGTLVTVSS

SEQ ID NO: 459 - protein, 126 aa, synthetic construct
  source 1..126: note = rbd.141 VH
EVQLVESGGGVVQPGRSLRLSCAASGFTFSSYGMHWVRQAPGKGLEWVAVISYDGKIKYYADSVEGRFTISRDNSKNALYLQMNSLRAEDTAVYYCAKYGIVTSFGVVPPYDMDVWGQGTTVTVSS

SEQ ID NO: 460 - protein, 129 aa, synthetic construct
  source 1..129: note = rbd.142 VH
EVQLVESGGGVVQPGRSLRLSCVASGLTFSRYGMHWVRQAPGKGLEWVAVTTYDGSNKDYADSVKGRFTISRDNSKNTLYLQMNSLRAEDTAVYYCAKVGGGEYCSGGSCYGSYGMDVWGQGTTVTVSS

SEQ ID NO: 461 - protein, 120 aa, synthetic construct
  source 1..120: note = rbd.143 VH
EVQLVESGGGLVQPGGSLRLSCSASGFTFRRYAMHWVRQAPGKGLQHVSAISSDGDNTHYADSVKGRFTISRDNFKNTLHLQMSSLRPEDTAVYYCVKDPFSNFHYFDCWGQGILVTVSS

SEQ ID NO: 462 - skipped

SEQ ID NO: 463 - protein, 110 aa, synthetic construct
  source 1..110: note = rbd.106 VL
QSVLTQPPSASGTPGQRVSISCSGSSSNIGSNSVNWYQQFPGTAPKLLMFSSYQRPSGVPDRFSGSKSGTSASLAISGLQSEDEADYYCAAWDDSLNGVVFGGGTKLTVL

SEQ ID NO: 464 - protein, 108 aa, synthetic construct
  source 1..108: note = rbd.110 VL
SYELTQPPSVSVAPGKTATITCGGNNIGSAGVHWYQQRPGQAPVLVVYDDSDRPSGIPERFSGSTSGNTATLTISRVEAGDEADYYCQVWDSNSAPVIFGGGTKLTVL

SEQ ID NO: 465 - protein, 111 aa, synthetic construct
  source 1..111: note = rbd.112 VL
QSVLTQPPSVSGAPGQRVTISCTGSSSNLGAGYDVHWYQQLPGTAPKLLIYANSNRPSGVPDRFSGSKSGTSASLAMTGLQAEDEADYYCQSYDSSLGGYVFGFGTKVTVL

SEQ ID NO: 466 - protein, 106 aa, synthetic construct
  source 1..106: note = rbd.113 VL
DIQMTQSPSTLSASVGDRVTITCRASQSINRWLAWYQQKPGKAPELLIYDASDLESGVPSRFSGSGSGTEFTLTISSLQPDDFATYYCQQYNSYWTFGQGTKVEIK

SEQ ID NO: 467 - protein, 107 aa, synthetic construct
  source 1..107: note = rbd.117 VL
DIQMTQSPSTLSASVGDRVTITCRASQSISSWLAWYQLKPGKAPKLLIYDASSLESGVPSRFSGSGSGTEFTLTISSLQPDDFATYYCQQYNTYPFSFGPGTKVDIK

SEQ ID NO: 468 - protein, 107 aa, synthetic construct
  source 1..107: note = rbd.123 VL
DIQMTQSPSTLSASVGDRVTITCRASQSISSWLAWYQQKPGKAPNLLIYDASSLESGVPSRFSGSGSGTEFTLTISSLQPDDFATYYCQQYKSYPYTFGQGTKLEIK

SEQ ID NO: 469 - protein, 112 aa, synthetic construct
  source 1..112: note = rbd.141 VL
DVVMTQSPLSLPVTLGQPASISCRSSQSLVYSDGNTFLNWFQQRPGQSPRRLIYKVSDRDSGVPDRFSGSGSGTDFTLKISRVEAEDVGVYYCMQGTNWPPTFGQGTKVEIK

SEQ ID NO: 470 - protein, 107 aa, synthetic construct
  source 1..107: note = rbd.142 VL
DIQMTQSPSSLSASVGDSVTITCRASQSISSYLNWYQQKPGKAPKLLIFAASSLESGVPSRFSGSGSGTDFTLTISSLQPEDFATYYCQQSYSTPYTFGQGTKLEIK

SEQ ID NO: 471 - protein, 107 aa, synthetic construct
  source 1..107: note = rbd.143 VL
DIQMTQSPSTLSASVGDRVTITCRASQSIRSWLAWYQQKPGKAPKLLIYDASSLESGVPSRFSGSGSGTEFTLTISSLQPDDFATYYCQQYNSYSSTFGQGTKVEIK